(19) United States
(12) Patent Application Publication
(10) Pub. No.: US 2021/0227809 A1
(43) Pub. Date: Jul. 29, 2021

(54) GENERATION OF HUMAN ENDODERMAL ORGANS IN PIG MODEL USING LINEAGE RESTRICTED ENDODERMAL PRECURSORS

(71) Applicant: UNIVERSITY OF MARYLAND, COLLEGE PARK, College Park, MD (US)

(72) Inventors: Chi-Hun Park, Laurel, MD (US); Bhanu Prakash V.L. Telugu, Columbia, MD (US)

(21) Appl. No.: 17/048,906

(22) PCT Filed: Apr. 17, 2019
(86) PCT No.: PCT/US2019/028001

§ 371 Date: Oct. 19, 2020

Related U.S. Application Data
(60) Provisional application No. 62/659,111, filed on Apr. 17, 2018.

(51) Int. Cl. A01K 67/027; C12N 15/90; C12N 5/073

(52) U.S. Cl. CPC A01K 67/0271; C12N 15/907; C12N 5/0605; A01K 2267/03; C12N 2517/10; A01K 2227/108; A01K 2207/12; C12N 2517/04

(57) ABSTRACT

Disclosed are methods of growing and culturing xenotypic tissue or xenotypic organs in a mammalian species. The methods of growing human organs in other mammalian species are disclosed and such human organs can be used for transplant purposes.

### CROSS-REFERENCE TO RELATED APPLICATIONS

This application claims benefit of U.S. Provisional Application No. 62/659,111 filed Apr. 17, 2018, which is hereby incorporated herein by reference in its entirety.

### BACKGROUND

In the United States alone, more than 123.000 men, women and children currently need lifesaving organ transplants (optn.transplant.hrsa.gov/). Every 10 minutes another name is added to the national organ transplant waiting list. Sadly, an average of 21 people die each day because the organs they need are not donated in time, with the numbers expected to increase every year. The ability to generate exogenous organs in pig for transplantation into humans (xenotransplantation) is considered as one of the sources to bridge this shortfall. The results that serve as a basis for the technology of exogenous organ development in this study are expected to bridge the gap in the understanding of the genetic basis of endodermal organ development, primarily pancreas and liver. Pig is already being used for xenotransplantation studies as the size of the animal, organs and physiology are similar to humans, making it an ideal animal model for investigation in this study. Additionally, there has been growing evidence to suggest that the genetic contribution to organogenesis as studied in mouse often has conflicting results in humans. This necessitates investigation in a higher phylogenetic species, such as pig. Described herein is the feasibility of generating organs of endodermal origin, in this case a vital organ such as pancreas or liver, from donor progenitor cells of embryonic origin called extraembryonic endodermal cells (XEN cells) or XEN-like cells from patient-specific stem cells. The XEN cells contribute to endodermal organ development including pancreas and liver, without contributing to other major lineages such as brain, gonads, skin, etc. These experiments will serve as a basis for the use of stem cells via a XEN like progenitors as donors in the future (FIG. 1).

### SUMMARY OF EMBODIMENTS

The disclosure relates to a method of creating xenotypic organ cells in an animal comprising: contacting a gene-modifying amino acid sequence and/or gene-modifying nucleic acid sequence with one or a plurality of XEN cells from a first species or one or a plurality of embryos from a second species for a time period sufficient to produce a genetic modification in a genome of the one or a plurality of XEN cells or the one or a plurality of embryos; (a) injecting the one or a plurality of XEN cells from one species into the one or a plurality of embryos; (b) implanting the embryo into a female host from the second species to produce a genetically modified fetus. The disclosure also relates to a method of creating xenotypic organ cells in an animal comprising: further comprising the steps of: allowing the embryo to develop into a fetus; and allowing the female host animal to deliver an infant animal comprising the one or a plurality of XEN cells after a period of time sufficient for the fetus to fully develop in the infant animal; or allowing the fetus to develop into an infant animal after a period of time sufficient to remove the fetus surgically from a womb of the female host animal and live ex utero. In some embodiments, the method further comprises the step of: screening the one or plurality of XEN cells and/or the one or plurality of embryos for a genetic modification after step (a). In some embodiments, the method further comprising the step of: allowing the infant animal to develop into an adult animal. In some embodiments, the gene-modifying amino acid sequence comprises one or a combination of functional amino acid sequences selected from: a CRISPR enzyme. TAL nuclease, zinc finger nuclease, and a transposon.

The disclosure relates to a method of growing a xenotypic organ or organ tissue in an animal comprising: (a) contacting a gene-modifying amino acid sequence and/or gene-modifying nucleic acid sequence with one or a plurality of mammalian embryos from one species for a time period sufficient to produce a genetic modification in a genome of the one or a plurality of embryos; and (b) injecting one or a plurality of XEN cells from a second species into an embryo of the first species. The method further comprises: (c) implanting the embryo into a female host from the first species after performance of step (b). The method of claim any of claims 16 further comprising the step of: (d) allowing a time period to elapse sufficient for an embryo to develop into a fetus within the female host after performance of step (c); and (e) allowing the female host animal to deliver an infant animal comprising the one or a plurality of XEN cells after a period of time sufficient for the fetus to fully develop as a fetus, or (e) allowing the fetus to develop into an infant animal after a period of time sufficient to remove the fetus surgically from a womb of the female host animal and live ex utero. In some embodiments, the methods further comprise the step of: screening the one or plurality of embryos for a genetic modification after step (a). In some embodiments, the methods further comprise the step of: (f) allowing the infant animal to develop into an adult animal.

The disclosure also relates to a method of microinjecting XEN cells and/or XEN-like cells from a first mammalian species into an embryo of a second mammalian species comprising: (a) harvesting XEN cells and/or XEN-like cells from a culture; (b) culturing the embryo; and (c) injecting the XEN cells and/or XEN-like cells into the embryo. In some embodiments, the first species is a primate and wherein the second species is a pig.

In some embodiments, the first species is a human. In some embodiments, the methods further comprise the step of culturing the XEN cells and/or XEN-like cells before steps (a) and (c).

In some other embodiments, the XEN cells and/or XEN-like cells are thawed from a frozen state before the step of culturing the XEN cells and/or XEN-like cells.

The disclosure also relates to a transgenic or chimeric animal and methods of making the same using any one or more steps disclosed above. In some embodiments, the transgenic animal or chimeric animal comprising tissues that are chimeric in respect to tissues of endodermal origin. In some embodiments, the chimeric or transgenic animal is chimeric in respect to certain organs, such as the liver or pancreas. In some embodiments, the chimeric or transgenic animal is a livestock animal comprising human tissue derived from endodermal embryonic cells. In some embodiments, the chimeric or transgenic animal is a livestock animal comprising human tissue derived from human XEN cells or XEN-like cells. In some embodiments, the chimeric or transgenic animal is a pig comprising chimeric organs, such as the liver or pancreas. In some embodiments, the chimeric or transgenic animal is a pig comprising humanized chimeric organs, such as a humanized liver or pancreas.

In some embodiments, any or all of the methods comprise a first species that is a human and a second species that is a livestock animal. In some embodiments, the methods disclosed herein relate to an embryo that is a pig or minipig. In some embodiments, the methods disclosed herein relate to one or a plurality of XEN or XEN-like cells that are derived from a human or are human.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-F show a schematic outlining the strategy for generation of human endodermal organs (e.g., liver, pancreas) in a pig bioreactor. (A, top) In vitro fertilized or parthenote embryos from discarded human oocytes can be used to establish (B) XEN cells in culture. Alternatively. (A, bottom) patient-specific induced pluripotent stem cells (iPSC) or multipotent stem cells can be differentiated into (B) XEN-cell like progenitor endodermal fate. (C) Porcine embryos that are either injected with CRISPR reagents (or other editors) that ablate specific endodermal gate-keeper genes or cloned embryos generated from cells lacking a gate keeper gene, could be (D) injected with human XEN cells or progenitor cells (blastocyst complementation) or at a later conceptus stage into liver or pancreatic primordia (fetal complementation), and, E) transplanted into surrogate animals. F) Following gestation, live pigs will be generated that carry the endodermal cell types and organs contributed from the donor human cells, which can be harvested for a multitude of applications as described above.

FIG. 2 shows a schematic diagram that the applications of the human cells and organs derived from pigs are numerous. The generation of pigs with transplantable human cells will feed into numerous biomedical platforms, including 3D-printing (liver, pancreas, kidney, bladder, etc.) and organ-on-chip applications. The pigs carrying human cells can be employed for pharmaceutical research including studying pharmacokinetics and pharmacodynamics, and toxicological evaluation of developmental drugs. Additionally, the availability of “on-demand” human cells can be used for cellular therapies. With the development of xenotransplantation research, where the pig genomes are being modified to tolerate immune-rejection, the human pig chimera approach can be utilized to generate transplantable solid “human” organs.

FIG. 3 shows the attachment and primary colony outgrowth of porcine blastocyst. These are day 7 porcine blastocysts that when plated on mitotically inactivated feeder cells establish primary outgrowths and colonies approximately 3-4 days after seeding. In the initial outgrowths, EPI: epiblast: TE: trophectoderm; and PE: primitive endoderm regions are clearly discernable.

FIG. 4 shows lineage-specific marker expression in primary colonies. Three early distinct lineages, epiblast (EPI depicted by SOX2 and NANOG), trophectoderm (TE, CDX2 and CK18), and primitive endoderm cells (PrE cells; GATA4, GATA6) are seen in the initial outgrowth at different time points during culture, which can be readily distinguished by morphological features and expression of known lineage-specific markers.

FIGS. 5A and 5B show the characterization of a representative XEN cell line. Immunocytochemical (A) and quantitative PCR (B) analyses confirmed that he XEN cells express high levels of endodermal lineage markers (GATA4, GATA6, and SOX17), and SALL4, which maines sternness of XEN cells. Additionally, definitive endodermal markers (HNF4 and FOXA2) are expressed likely showing their propensity to differentiate into committed endodermal cells. The other lineage markers (SOX2, NANOG, CDX2. HAND1) with the exception of EOMES-a TE marker, which was reported to be expressed in the rat XEN cells. Relative expression of candidate genes relative to the yolk-sac was shown in qPCR, confirming their XEN cell origin.

FIG. 6 shows spontaneous and directed differentiation of XEN cell lines. XEN cells in monolayer or embryoid bodies can be directed to differentiate into visceral endoderm (VE) or primitive endoderm (PE) of yolk sac.

FIG. 7 shows generating live animals using XEN GFP-Col-KI cells as a nuclear donor.

FIGS. 8A-8C show CRISPR cas9-mediated HDR (A), cloning efficiency of XEN cells and fetal fibroblasts (B), and live offspring generated by cloning of GFP:XEN cells (C). NGN3 represents pigs cloned from fetal fibroblasts. Ossabaw XEN: piglets cloned from XEN cells. Live XEN cell derived piglets are shown. The piglets express GFP under blue light. Internal organs also express GFP

FIGS. 9A-9D show the contribution of EGFP-expressing XEN Cells to chimeras following blastocyst complementation. A) Schematic showing the injection of XEN cells that constitutively express GFP into parthenogenic pig embryos. B) Bright field and fluorescent merged images of a (a) GFP cloned blastocysts and its derivative a (b) GFP+XEN cells, and (c) a GFP+XEN-injected pig blastocyst at day 5. (d) Morphologically normal embryonic chimera at day 21, (e.f) yolk sac, (g) allantois containing GFP-positive XEN derivatives. C) Table showing relative efficiencies of chimeric potential of GFP expressing XEN cells when injected into D5 parthenotes and following transplantation. Based on results from two embryo transfer trials transferring 30 or 36 blastocyts into surrogate sow, we have noticed 60-70% of the embryos showing chimerism, D) with a total contribution of injected cells at 11% in the resulting fetuses.

FIG. 10 shows XEN cells contribute to extra-embryonic membranes. True to their source of derivation and their name, the XEN cells contribute to extra-embryonic membranes the Amnion and allantochorion.

FIGS. 11A-11C show XEN cells contribute to endodermal cells (Liver and pancreas) in chimeric fetus. A) A sagittal-section of Hematoxylin and Eosin (H&E) stained XEN cells injected Day 21 chimeric parthenote fetuses. B) an immune-histochemistry image of GFP cells probed with anti-GFP antibodies and stained with secondary HRP conjugated antibody, showing extensive chimerism to the endodermal derivatives, as indicated by C) staining with GATA6 and SALL4.

FIGS. 12A-12I show distinct subpopulations arise from the porcine blastocyst outgrowths. (a) Phase contrast images depicting morphologies of embryonic outgrowths from days 2 to 5 in culture. In the figure EPI, TE and PrE stands for epiblast, trophectoderm and primitive endoderm, respectively. (b) Immunostaining for key transcription factors, SOX2 and NANOG (ICM), CDX2 and CK18 (TE), and GATA6 (PrE) in the primary outgrowth at day 3 after explants. (c) Representative immunofluorescence images of late blastocyst (ICM in dotted circle). In the figure, fraction of cells and percentage of cells that stained positive for NANOG or SOX2 was shown. (d) The bar graph showing the attachment and outgrowth rates of early and late blastocysts. (e) Frequencies of SOX2- and GATA6-positive cells in outgrowths. ND: not detected. (f) Representative immunostaining (top) and quantitation (bottom) of the number of NANOG or GATA4 positive nuclei in primary outgrowths cultured for 7 days. Open and solid arrows indicate NANOG/GATA4 co-positive and GATA4 positive only cells, respectively. (g) Representative fluorescence images of CK18 and GATA4 of a Day 7 primary outgrowth (right). Comparison of the transcriptional levels of selected lineage marker genes between PrE cells and EPI cells by qPCR;*, p<0.05 according to unpaired t test; error bars represent SEM (n=3) (left). ACTB was used as an endogenous control. (h) The expression of H3K27me3 and SALL4 in day 7 primary outgrowth (right). Inset shows the zoom-in of the dashed box. The bar graph showing the quantitation of the percentage of H3K27me3 focal dots in SALL4 positive or negative cells (left). In all images, nuclei were counterstained with DAPI. Scale bar: 100 μm. (i) The relative XIST mRNA levels in PrE cells compared to EPI cells: *, p<0.05 according to unpaired t test; error bars represent SEM (n=3). ACTB was used as a loading control.

FIGS. 13A-13N show the establishment and characterization of pXEN cells (a) Representative bright-field images of EPI-derived primary colonies, and PrE-derived XEN cells at passages 3-5. (b) Efficiency of colony formation of pXEN cells passaged as clumps or single cells. The colony forming activity were greatly impaired when dissociated as single cells. Cells were passaged as clumps by mechanical (clumps-me) or enzymatic dissociation (clumps-en) with Accutase. (c) Alkaline phosphatase (ALP) staining of an in vivo-derived pXEN cells (Xv#9) after culturing for 3 and 7 days. (d) Representative fluorescence images of VIMENTIN (red) and AFP (green) (e) Expression of the indicated markers in pXEN at passages 30-35. (f) Effect of growth factors supplementation on PrE derivation. pXEN cells were seeded onto a 6-well-plate seeded containing a density of 5×104 feeder cells per cm2, and (g) cell number estimated 48 h following passage. Data are is presented as means±s.d. (n=3). (h) qPCR analyses of total RNA isolated from pXEN cells grown in either the presence or absence of LIF/bFGF for 4 days. ACTB was used as a loading control. The values are represented as mean±s.d. (n=3). (i) Representative images of pXEN cells show the expression of stem cell marker, SALL4 (green) that are significantly reduced in the cells that had lost lipid droplet. Scale bar: 100 μm. (j) qPCR analysis of pXEN cells derived from different embryonic origins. ACTB was used as a loading control. The values are represented as mean s.d. (n=3). (k) Representative karyotypic analysis of pXEN cell lines, with numbered chromosomes. (1) RNA-seq analysis of pXEN cells and comparison with analogous derivatives. Data from pig XEN cell lines as well as published data on related cell lines (mouse and rat XEN cells) were included in the comparison. Principal component analysis (PCA) plot of two pXEN cells and other samples. Upper inset shows the color code for each cell type, lower inset shows a separate PCA of only pig vs. mouse vs. rat XEN cells. (m) hierarchical clustering of pXEN and related samples. (n)Heatmap comparison of selected XEN-associated extraembryonic endodermal (ExEn) marker gene expression of all samples.

FIGS. 14A-14E show chimeric contribution of pXEN cells to embryonic and extraembryonic lineages in post-implantation Day 21 embryos. (a) Schematic representation of the chimera assay. (b) Table presents a summary of chimera experiments performed by injection of pXEN cells into blastocysts. In the Table, Ys: yolk sac; ExE: extraembryonic membranes; N/D: not defined (severely retarded fetuses with no fetal or yolk sac parts); and “*” stands for the embryos at the pre-attachment stages (spherical or ovoid). (c) Representative bright field and fluorescence merged images of normal (XeC#2-3 and XeC#2-4) and retarded (XeC#2-6) fetuses at day 21 of gestation. Yolk sac outlined by the dashed line, and enlarged view of the region marked by the dashed box is shown in the right. In the figure Al stands for allantois; Ch, chorion; Emb, embryo; Ys, yolk sac. (d) Bar graph representing percent contributions of GFP-XEN in chimeras determined by qPCR; *, p<0.05 according to unpaired t test; error bars represent±SEM (¬¬n=3). (e) Representative sagittal or transverse sections of fetuses showing dual immunofluorescence staining for GFP (green) and SALL4 or PECAM1 (red) in embryos; the arrows indicate GFP-positive cells derived from injected pXEN cells in sections. Inset are zoom-in magnified images of the dashed box. Nuclei were stained with DAPI (blue). Al, allantois; Ch, chorion; Emb, embryo; Lp, liver primordium; Pg primitive gut; Ys, yolk sac; Am, amnion; Hp, heart primordium; So, somite. Scale bar: 100 μm.

FIGS. 15A-15C show the generation of viable cloned piglets using pXEN or fibroblasts. (a) Summarv of SCNT experiments. #Cloning efficiency was obtained by calculating total no. fetuses or piglets/total no. embryos transferred. $data obtained from our previous study. *NGN3−/− cells originated from our previous report25. All the fetal fibroblasts and pXEN cells with the exception of NGN3−/− cells used as SCNT donors were derived from the same fetus (female Ossabow fetal fibroblast #6). (b) Representative images showing 10 days old NGN3 KO white (outbred)- and XEN Black (Ossabow)-coated littermates. The fluorescence images of live GFP+ piglets and whole organs taken with blue light illumination showing ubiquitous expression of GFP transgene, and confirming the pXEN cell as nuclear donors. (c) A representative digital gel image of the 1.2-kb amplicon with primers within and outside of the targeting vector confirming site-specific knockin was generated by Fragment Analyzer.

FIGS. 16A-16E shows distinct subpopulations arise from the blastocysts outgrowth. a. Phase contrast images and immunostaining of the primary outgrowth. In the primary outgrowth, GATA-positive large (filled arrowhead) and small (open arrowhead) round cells, and CDX2-positive trophoblast cells (filled arrow) were observed. b. Representative fluorescence images of CK18 in the blastocyst (ICM in dotted circle) and the primary outgrowth showing mixed populations, including large (rPrE) and small (sPrE) round cells. c, Representative fluorescence images of selected PrE markers in in vitro Day 7 blastocysts. d, Representative immunostaining and e, quantitation of the number of SOX2-positive nuclei in primary outgrowths cultured for 7 days.

FIGS. 17A-H show self-renewal of extra-embryonic endoderm XEN cells. a. Representative bright-field images showing separation of PrE cells from the primary outgrowth after 7-9 days of culture. b, Efficiency of colony formation of pXEN cells passaged as clumps by mechanical (Mec) or enzymatic dissociation with Accutase (Acc), Collagenase IV (Col), Dispase (Dis), and Trypsin 0.5% (Try) in the presence or absence of ROCK inhibitor (Y-27632). c, Representative images of pXEN cells show the expression of proliferation marker, PCNA (right). d, Expression of the indicated markers in pXEN at passages 30-35. e. Effect of culture medium during propagation of pXENs. The cells were seeded in 6-well-plates at a density of 5×104 cells per cm2 and estimated 48 h after a lag period following passage. Data are presented as means±s.d. (n=3). f, Representative bright-field images of pXEN in different culture mediums. g, qPCR analyses with total RNA isolated from pXEN cells grown in either the presence or absence of LIF/bFGF for 4 days. ACTB was used as a loading control. The values are represented as mean s.d. (n=3). h, Expression of the indicated markers in pXEN cells. Two subpopulation frequently observed during propagation of pXENs.

FIGS. 18A-18C show chimeric contributions of pXEN cells in embryo. a. Representative images of generation of GFP-labeled XEN (filled arrow) cell line and chimeric embryos. b, Representative sagittal sections showing hematoxylin and eosin (H&E) stains and immunofluorescence for GFP (green) and SALL4 (red) in a chimeric D21 yolk sac. In the left panel, the yolk sac consists of 3 thin cellular; the inner surface of the mesothelium (Me) the outer layer of visceral endoderm (Ve), the yolk sac cavity with primitive erythrocytes (Er) surrounded by a layer of endothelial cells. In the right panel, section was immunostained with anti-GFP (green) and anti-SALL4 antibodies, which were present in the visceral endodermal layers (dotted line). A few GPF-positive cells were observed in the primitive erythrocytes (arrow). c. Section was immunostained with anti-GFP (green; arrow) and anti-PECAM1 antibodies showing that cells from GFP-pXEN contribute to embryonic tissues and fetal membranes in a D21 chimera (#1-2). The area in the dashed box are displayed at a higher magnification. Nuclei were stained with DAPI (blue). Ch, chorion; Lp, liver primordium: Ys, yolk sac: Am, amnion, Op otic pit

### DETAILED DESCRIPTION

The disclosed method and compositions may be understood more readily by reference to the following detailed description of particular embodiments and the Example included therein and to the Figures and their previous and following description.

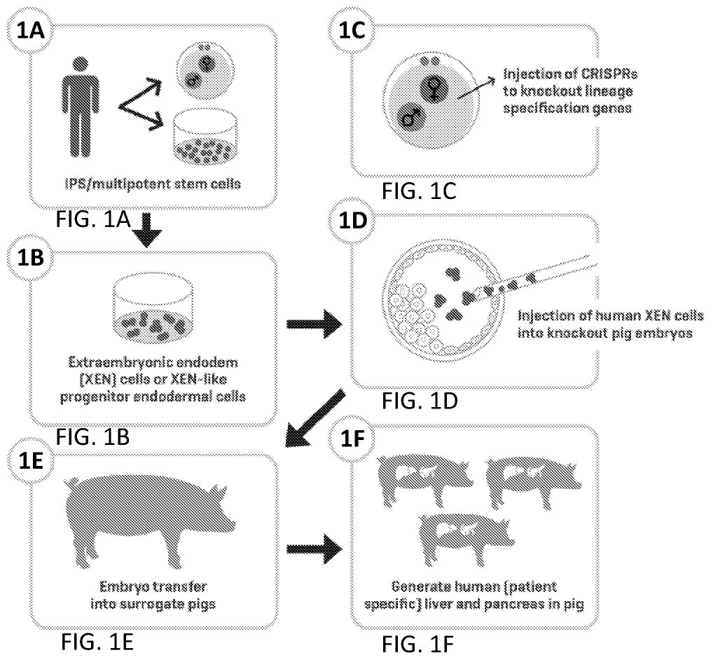

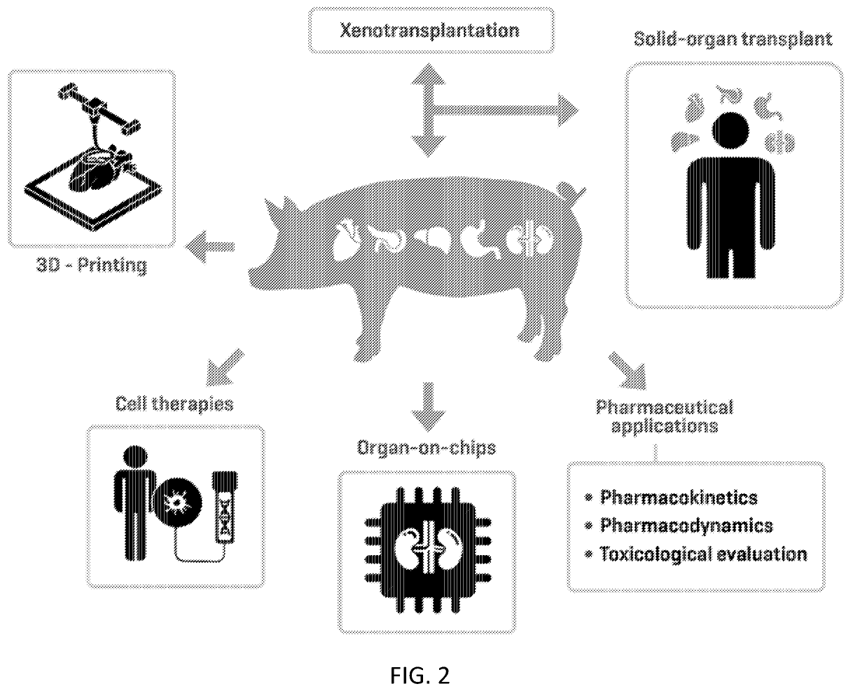

Here, we developed an alternative method that utilizes the robustness of embryo injections, including the ease of access to target genes and high frequency of edits, with high predictability of establishing a cohort of genome edited animals, characteristic of somatic cell nuclear transfer (SCNT) (FIG. 1).

Briefly, in vitro or in vivo fertilized zygotes can be microinjected for achieving targeted genetic modification, plated onto mitotically inactivated feeders to establish epiblast derived primary embryonic fibroblasts (EF) or extraembryonic endodermal (XEN) cells. The latter, which are the derivatives of the primitive endoderm can be maintained over extended periods of time (>40 passages) with no signs of senescence, prescreened for targeted modification and utilized to generate genome edited (GE) pigs with desired genetic modification.

With the ability to culture embryos, establish primary cells and screen for genotypes it is possible to perform genetic selection in vitro. In cattle and other livestock breeding systems, the genetic selection is primarily performed in male offspring, and the semen from the animals used for artificial insemination (AI) for rapid dissemination of genetics. That said, meiotic recombination during gametogenesis yields haplotypes (segments of genomes) that are inherited as a unit. Rare segregation events that result in optimal genome breeding value is often left to chance and the offspring after a prolonged gestation period will have to be screened to identify the right combination of haplotypes. By fertilizing oocytes and establishing embryos and primary cultures in vitro, the cells can be pre-screened, and the cells with optimal breeding value can be used for generating offspring. This will be of tremendous value to the livestock genetics industry. The ability to perform genome editing in embryos and prescreen for correct mutations in vitro prior to generating offspring will also be of tremendous value for the biomedical sector, where animals with particular mutations are often desired.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a XEN cell” includes a plurality of such cells, reference to “the XEN cell” is a reference to one or more XEN cells and equivalents thereof known to those skilled in the art, and so forth.

Unless otherwise indicated, the cell culture and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991). D. M. Glover and B. D. Hames (editors), DNA Cloning: A Practical Approach, Volumes 1-4, IRL Press (1995 and 1996), and F. M. Ausubel et al., (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al., (editors) Current Protocols in Immunology, John Wiley & Sons (including all updates until present).

“Embryo” is a multicellular diploid eukaryote in early stage of development. In some embodiments, the embryo is a pig, goat, sheep, horse, cow, dog, cat, camel, rat or mouse embryo. In some embodiments, the embryo is a pig embryo comprising one or a plurality of XEN cells. In some embodiments, the embryo is a pig embryo in a blastocyst stage. In some embodiments, the embryo is a mammalian embryo in a blastocyst stage. In some embodiments, the embryo is a pig embryo in a blastocyst stage into which an XEN cell is injected.

“Embryonic stem cell” or ES cell is a pluripotent cell derived from the inner mass of the blastocyst or early stage embryo.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed”. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “gene” means a DNA sequence that codes for or corresponds to a particular sequence of amino acids which comprise all or part of one or more proteins or enzymes, and may or may not include introns and regulatory DNA sequences, such as promoter sequences, 5′-untranslated region, or 3′-untranslated region which affect for example the conditions under which the gene is expressed. Some genes may be transcribed from DNA to RNA, but are not translated into an amino acid sequence. Other genes may function as regulators of structural genes or as regulators of DNA transcription.

“Genetically modified” or “genetic modification” means a gene or other DNA sequence that is altered from its native state (e.g. by insertion mutation, deletion mutation, nucleic acid sequence mutation, truncation or other mutation), or that a gene product is altered from its natural state (e.g. by delivery of a transgene that works in trans on a gene's encoded mRNA or protein, such as delivery of inhibitory RNA or delivery of a dominant negative transgene). In some embodiments, the genetic modification is a modification of genomic DNA or RNA transcripts by delivering an enzyme capable of gene editing optionally with one or more template nucleic acid sequences that (i) knocks in or activates expression of a native gene expressed at a level higher than it is expressed endogenously: or (ii) knocks out or inactivates/inhibits expression of a native gene to a level of expression lower than it is expressed endogenously. In some embodiments a cell that is designated −/− when referring to a gene means that a gene or material portion of a gene is physically removed from the genome of the cell such that there is no expression of the encoded protein corresponding to the gene. In some other embodiments, a cell that is designated −/− when referring to a gene means that a gene or material portion of a gene is modified such that the physical gene is present within the genome of the cell but there is no expression of a biologically functional encoded protein corresponding to the gene; or there is limited or low expression of a functional protein corresponding to the gene such that the amount of functional protein is ineffective at causing biological activity, An example of this is basal or lower level protein expression of a gene that does not cause a biological effect.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g. fused cells, genetically modified cells, etc.).

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed. In some aspects, about refers to +/−10%, more preferably +1-5%, more preferably +/−2.5%, even more preferably +/−1%, of the designated value.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to” or “including, without limitation.”

The term “or” is used herein to mean, and is used interchangeably with, the term “and/or,” unless context clearly indicates otherwise. For example, a nucleoside with a modified base or a modified sugar is understood to include the options of a nucleoside with a modified base, a nucleoside with a modified sugar, and a nucleoside with a modified base and a modified sugar.

The term “about” is used herein to mean within the typical ranges of tolerances in the art. For example, “about” can be understood as about 2 standard deviations from the mean. According to certain embodiments, about means+/−10%. According to certain embodiments, about means+/−5%, +/−2%, or +/−1%. When about is present before a series of numbers or a range, it is understood that “about” can modify each of the numbers in the series or range.

The term “at least” prior to a number or series of numbers (e.g. “at least two”) is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, “up to” as in “up to 10” is understood as up to and including 10, i.e., 0, 1, 2, 3, 4, 5, 6, 7, 8, 9, or 10.

Ranges provided herein are understood to include all individual integer values and all subranges within the ranges.

As used herein, the term “in combination with,” is not intended to imply that the therapy or the therapeutic agents must be administered at the same time and/or formulated for delivery together, although these methods of delivery are within the scope described herein. The therapeutic agents can be administered concurrently with, prior to, or subsequent to, one or more other additional therapies or therapeutic agents.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Non-limiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia et al., J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

The term “and/or”, e.g., “X and/or Y” shall be understood to mean either “X and Y” or “X or Y” and shall be taken to provide explicit support for both meanings or for either meaning.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply. “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, intemucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

In one embodiment, the substitutions made within a heavy or light chain that is at least 95% identical (or at least 96% identical, or at least 97% identical, or at least 98% identical, or at least 99% identical) are conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine: (6) acidic side chains: aspartate and glutamate, and (7) sulfur-containing side chains are cysteine and methionine.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4: 11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix and a gap weight of 16, 14, 12, 10, 8. 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the molecules of the invention. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs {e.g., XBLAST and NBLAST) can be used. (See www.ncbi.nlm.nih.gov).

### B. Extra-Embryonic Endoderm CeUs (XEN CeUs)

In certain aspects the present disclosure relates to isolated cells that have developed from a zygote, for example a zygote that has been generated or isolated in vitro. In some embodiments, the isolated cell line is an extraembryonic endodermal (XEN) cell line. The in vitro zvgote may be cultured for several days to generate the XEN cell line. For example, in some embodiments, the zygote is cultured in vitro for at least 1, 2, 3, 4, 5, 6, 7, 8, 9, or 10 days to generate the XEN cell line. Any of these values may be used to define a range for the number of days that the zygote is cultured in vitro. For example, the zygote may be cultured for 1 to 10 days, for 4 to 6 days, or from 4 to 10 days. In some aspects, disclosed are compositions comprising one or more XEN cells.

In some aspects, multipotent stem cells or induced pluripotent stem (iPS) cells can be used to produce XEN cells or XEN-like cells. In some embodiments, the XEN or XEN-like cells can be isolated or derived from a culture after about 10, 20, 30, 40, 50, 60, 70, 80, 90, 100, 110, 150, 200, 250, 300, 350, or 365 days.

The disclosed XEN cells can be identified by a particular expression profile of marker genes. For example, in some embodiments, a XEN cell line expresses one or more of GATA4, FOXA2, GATA6 and SOX17. In some embodiments, a XEN cell line does not express or is deficient in expression of one or more of CDX2, NANOG, SOX2. In a particular embodiment, a XEN cell line expresses GATA4, FOXA2, GATA6 and SOX17, and does not express CDX2, NANOG, SOX2. XEN-like cells are used herein to mean cells that are modified to express one or more of GATA4, FOXA2, GATA6 and SOX17, but are deficient or substantially deficient in expression of at least one CDX2, NANOG, SOX2 as compared to unmodified iPS cells. In some embodiments, a XEN-like cell is expresses one or more of GATA4. FOXA2, GATA6 and SOX17 but does not share the same morphological or functional characteristics of an XEN-cell.

In some aspects, the cells described herein can be from a mammal, for example a human, a non-human mammal, a primate, a sheep, a goat, a cow, a pig, llama, camel, rabbit or a horse.

Also disclosed are compositions comprising one or more XEN cells. In some embodiments, the disclosure relates to transgenic or chimeric animals comprising one or more XEN cells or one or more XEN-like cells derived from or from another species. Disclosed are compositions comprising chimeric tissues disclosed herein. In some embodiments, these chimeric tissues are suitable for xenotransplants.

### C. Methods

The disclosure relates to a method of creating xenotypic organ cells in an animal comprising: contacting a gene-modifying amino acid sequence and/or gene-modifying nucleic acid sequence with one or a plurality of XEN cells from a first species or one or a plurality of embryos from a second species for a time period sufficient to produce a genetic modification in a genome of the one or a plurality of XEN cells or the one or a plurality of embryos; (a) injecting the one or a plurality of XEN cells from one species into the one or a plurality of embryos; (b) implanting the embryo into a female host from the second species to produce a genetically modified fetus. The disclosure also relates to a method of creating xenotypic organ cells in an animal comprising: further comprising the steps of: allowing the embryo to develop into a fetus; and allowing the female host animal to deliver an infant animal comprising the one or a plurality of XEN cells after a period of time sufficient for the fetus to fully develop in the infant animal; or allowing the fetus to develop into an infant animal after a period of time sufficient to remove the fetus surgically from a womb of the female host animal and live ex utero. In some embodiments, the method further comprises the step of: screening the one or plurality of XEN cells and/or the one or plurality of embryos for a genetic modification after step (a). In some embodiments, the method further comprising the step of: allowing the infant animal to develop into an adult animal. In some embodiments, the gene-modifying amino acid sequence comprises one or a combination of functional amino acid sequences selected from: a CRISPR enzyme, TAL nuclease, zinc finger nuclease, and a transposon.

The disclosure relates to a method of growing a xenotypic organ or organ tissue in an animal comprising: (a) contacting a gene-modifying amino acid sequence and/or gene-modifying nucleic acid sequence with one or a plurality of mammalian embryos from one species for a time period sufficient to produce a genetic modification in a genome of the one or a plurality of embryos; and (b) injecting one or a plurality of XEN cells from a second species into an embryo of the first species. The method further comprises: (c) implanting the embryo into a female host from the first species after performance of step (b). The method of claim any of claims 16 further comprising the step of: (d) allowing a time period to elapse sufficient for an embryo to develop into a fetus within the female host after performance of step (c); and (e) allowing the female host animal to deliver an infant animal comprising the one or a plurality of XEN cells after a period of time sufficient for the fetus to fully develop as a fetus, or (e) allowing the fetus to develop into an infant animal after a period of time sufficient to remove the fetus surgically from a womb of the female host animal and live ex utero. In some embodiments, the methods further comprise the step of: screening the one or plurality of embryos for a genetic modification after step (a). In some embodiments, the methods further comprise the step of: (f) allowing the infant animal to develop into an adult animal.

The disclosure also relates to a method of microinjecting XEN cells and/or XEN-like cells from a first mammalian species into an embryo of a second mammalian species comprising: (a) harvesting XEN cells and/or XEN-like cells from a culture; (b) culturing the embryo; and (c) injecting the XEN cells and/or XEN-like cells into the embryo. In some embodiments, the first species is a primate and wherein the second species is a pig. In some embodiments, the first species is a human. In some embodiments, the methods further comprise the step of culturing the XEN cells and/or XEN-like cells before steps (a) and (c). In some other embodiments, the XEN cells and/or XEN-like cells are thawed from a frozen state before the step of culturing the XEN cells and/or XEN-like cells.

The disclosure also relates to a transgenic or chimeric animal and methods of making the same using any one or more steps disclosed above. In some embodiments, the transgenic animal or chimeric animal comprising tissues that are chimeric in respect to tissues of endodermal origin. In some embodiments, the chimeric or transgenic animal is chimeric in respect to certain organs, such as the liver or pancreas. In some embodiments, the chimeric or transgenic animal is a livestock animal comprising human tissue derived from endodermal embryonic cells. In some embodiments, the chimeric or transgenic animal is a livestock animal comprising human tissue derived from human XEN cells or XEN-like cells. In some embodiments, the chimeric or transgenic animal is a pig comprising chimeric organs, such as the liver or pancreas. In some embodiments, the chimeric or transgenic animal is a pig comprising humanized chimeric organs, such as a humanized liver or pancreas.

In some embodiments, any or all of the methods comprise a first species that is a human and a second species that is a livestock animal. In some embodiments, the methods disclosed herein relate to an embryo that is a pig or minipig. In some embodiments, the methods disclosed herein relate to one or a plurality of XEN or XEN-like cells that are derived from a human or are human. In some embodiments the XEN or XEN-like cells express an amino acid sequence that comprises at least about 70%, 75%, 80%, 85%, 90%, 95%, 96%, 97%, 98%, 99%, or 100% sequence identity to one or more of the following at levels that are equal or exceed expression levels of cells that are not mutated and/or transformed:

GATA4
MYQSLAMAANHGPPPGAYEAGGPGAFMHGAGAASSPVYVPTPRVPSSVL
GLSYLQGGGAGSASGGASGGSSGGAASGAGPGTQQGSPGWSQAGADGAA
YTPPPVSPRFSFPGTTGSLAAAAAAAAAREAAAYSSGGGAAGAGLAGRE
QYGRAGFAGSYSSPYPAYMADVGASWAAAAAASAGPFDSPVLHSLPGRA
NPAARHPNLDMFDDFSEGRECVNCGAMSTPLWRRDGTGHYLCNACGLYH
KMNGINRPLIKPQRRLSASRRVGLSCANCQTTTTTLWRRNAEGEPVCNA
CGLYMKLHGVPRPLAMRKEGIQTRKRKPKNLNKSKTPAAPSGSESLPPA
SGASSNSSNATTSSSEEMRPIKTEPGLSSHYGHSSSVSQTFSVSAMSGH
GPSIHPVLSALKLSPQGYASPVSQSPQTSSKQDSWNSLVLADSHGDIIT
A
FOXA2
MLGAVKMEGHEPSDWSSYYAEPEGYSSVSNMNAGLGMNGMNTYMSMSAA
AMGSGSGNMSAGSMNMSSYVGAGMSPSLAGMSPGAGAMAGMGGSAGAAG
VAGMGPHLSPSLSPLGGQAAGAMGGLAPYANMNSMSPMYGQAGLSRARD
PKTYRRSYTHAKPPYSYISLITMAIQQSPNKMLTLSEIYQWIMDLFPFY
RQNQQRWQNSIRHSLSFNDCFLKVPRSPDKPGKGSFWTLHPDSGNMFEN
GCYLRRQKRFKCEKQLALKEAAGAAGSGKKAAAGAQASQAQLGEAAGPA
SETPAGTESPHSSASPCQEHKRGGLGELKGTPAAALSPPEPAPSPGQQQ
QAAAHLLGPPHHPGLPPEAHLKPEHHYAFNHPFSINNLMSSEQQHHHSH
HHHQPHKMDLKAYEQVMHYPGYGSPMPGSLAMGPVTNKTGLDASPLAAD
TSYYQGVYSRPIMNSS
GATA6
MALTDGGWCLPKRFGAAGADASDSRAFPAREPSTPPSPISSSSSSCSRG
GERGPGGASNCGTPQLDTEAAAGPPARSLLLSSYASHPFGAPHGPSAPG
VAGPGGNLSSWEDLLLFTDLDQAATASKLLWSSRGAKLSPFAPEQPEEM
YQTLAALSSQGPAAYDGAPGGFVHSAAAAAAAAAAASSPVYVPTTRVGS
MLPGLPYHLQGSGSGPANHAGGAGAHPGWPQASADSPPYGSGGGAAGGG
AAGPGGAGSAAAHVSARFPYSPSPPMANGAAREPGGYAAAGSGGAGGVS
GGGSSLAAMGGREPQYSSLSAARPLNGTYHHHHHHHHHHPSPYSPYVGA
PLTPAWPAGPFETPVLHSLQSRAGAPLPVPRGPSADLLEDLSESRECVN
CGSIQTPLWRRDGTGHYLCNACGLYSKMNGLSRPLIKPQKRVPSSRRLG
LSCANCHTTTTTLWRRNAEGEPVCNACGLYMKLHGVPRPLAMKKEGIQT
RKRKPKNINKSKTCSGNSNNSIPMTPTSTSSNSDDCSKNTSPTTQPTAS
GAGAPVMTGAGESTNPENSELKYSGQDGLYIGVSLASPAEVTSSVRPDS
WCALALA
SOX17
MSSPDAGYASDDQSQTQSALPAVMAGLGPCPWAESLSPIGDMKVKGEAP
ANSGAPAGAAGRAKGESRIRRPMNAFMVWAKDERKRLAQQNPDLHNAEL
SKMLGKSWKALTLAEKRPFVEEAERLRVQHMQDHPNYKYRPRRRKQVKR
LKRVEGGFLHGLAEPQAAALGPEGGRVAMDGLGLQFPEQGFPAGPPLLP
PHMGGHYRDCQSLGAPPLDGYPLPTPDTSPLDGVDPDPAFFAAPMPGDC
PAAGTYSYAQVSDYAGPPEPPAGPMHPRLGPEPAGPSIPGLLAPPSALH
VYYGAMGSPGAGGGRGFQMQPQHQHQHQHQHHPPGPGQPSPPPEALPCR
DGTDPSQPAELLGEVDRTEFEQYLHFVCKPEMGLPYQGHDSGVNLPDSH
GAISSVVSDASSAVYYCNYPDV

In some embodiments the XEN or XEN-like cells are free of or do not express or are deficient in expression of an amino acid sequence that comprises at least about 70%, 75%, 80%, 85%, 90%, 95%, 96%, 97%, 98%, 99%, or 100% sequence identity to one or more of the following at levels that are equal or exceed expression levels of cells that are not mutated and/or transformed:

CDX2
MYVSYLLDKDVSMYPSSVRHSGGLNLAPQNFVSPPQYPDYGGYH
VAAAAAAAANLDSAQSPGPSWPAAYGAPLREDWNGYAPGGAAAAANAVA
HGLNGGSPAAAMGYSSPADYHPHHHPHHHPHHPAAAPSCASGLLQTLNP
GPPGPAATAAAEQLSPGGQRRNLCEWMRKPAQQSLGSQVKTRTKDKYRV
VYTDHQRLELEKEFHYSRYITIRRKAELAATLGLSERQVKIWFQNRRAK
ERKINKKKLQQQQQQQPPQPPPPPPQPPQPQPGPLRSVPEPLSPVSSLQ
ASVSGSVPGVLGPTGGVLNPTVTQ
NANOG
KASAPTYPSLYSSYHQGCLVNPTGNLPMWSNQTWNNSTWSNQTQNIQSW
SNHSWNTQTWCTQSWNNQAWNSPFYNCGEESLQSCMQFQPNSPASDLEA
ALEAAGEGLNVIQQTTRYFSTPQTMDLFLNYSMNMQPEDV
SOX2
MYNMMETELKPPGPQQTSGGGGGNSTAAAAGGNQKNSPDRVKRPMNAFM
VWSRGQRRKMAQENPKMHNSEISKRLGAEWKLLSETEKRPFIDEAKRLR
ALHMKEHPDYKYRPRRKTKTLMKKDKYTLPGGLLAPGGNSMASGVGVGA
GLGAGVNQRMDSYAHMNGWSNGSYSMMQDQLGYPQHPGLNAHGAAQMQP
MHRYDVSALQYNSMTSSQTYMNGSPTYSMSYSQQGTPGMALGSMGSVVK
SEASSSPPVVTSSSHSRAPCQAGDLRDMISMYLPGAEVPEPAAPSRLHM
SQHYQSGPVPGTAINGTLPLSHM

### D. Transgenic Animals Used for Making XEN-derived Chimeras

The present invention relates to methods for producing a non-human animal, e.g. a sheep, goat, cow, pig or horse, comprising a targeted germline genetic modification. As used herein, the term “targeted germline genetic modification” refers to any genetic modification, such as but not limited to deletion, substation or insertion, made by way of human intervention at a predetermined location in the genome.

In one embodiment, the genetic modification results in reduced expression of one or more genes and/or proteins in the animal and/or progeny thereof. Thus, in this embodiment, a gene knockout animal can be produced. As used herein. “reduced” or “deficient expression” of one or more genes and/or proteins is meant that the translation of a polypeptide and/or transcription of a gene in the cells of an animal produced using the methods of the invention, or progeny thereof, is reduced at least 10%, or at least 20%, or at least 30%, or at least 40%, or at least 50%, or at least 60%, or at least 70%, or at least 80%, or at least 90%, or at least 95%, or at least 98%, or at least 99%, or 100% relative to an isogenic animal lacking the genetic modification. In some aspects there is 100% no residual expression of a knocked out gene. In some aspects, less than 100% expression of the knocked out gene can occur but the resulting expression does not lead to a functional protein.

In some aspects, the disclosed transgenic animals can have one or more genes knocked out in the endodermal development pathway. In particular, one or more genes involved in liver or pancreas development can be knocked out. For example, one or more of the following genes can be knocked out in the disclosed transgenic animals: FOXA2; GATA; BRY (Mesendoderm); FOXA2; GATA (Defenitive endoderm); SOX17: HHEX: GAT (Hepatopancreatic progenitor); PDXI (Pancreatic progenitor); NGN3 (Pancreatic endocrine progenitor); HNFI beta: HNF4alpha (Hepatoblast); HNF6; SOX9; HNFlbeta (Cholangiocyte) PROX1; and HNF4alpha (Hepatocyte). In some aspects, the one or more genes disclosed herein can be inactivated or deleted.

Animals produced using the methods of the invention can be screened for the presence of the targeted germline genetic modification. This step can be performed using any suitable procedure known in the art. For instance, a nucleic acid sample, such as a genomic DNA sample, can be analyzed using standard DNA amplification and sequencing procedures to determine if the genetic modification is present at the targeted site (locus) in the genome. In some embodiments, the screening also determines whether the animal is homozygous or heterozygous for the genetic modification.

In some aspects, the genetically modified animals are transgenic animals and comprise one or more of the XEN cells described herein. In some aspects, the screening method for identifying those animals that comprise the XEN cells is part of the experimental design. Non-human embryos are genetically modified, as described herein, and injected with human XEN cells. Only the embryos that comprise the XEN cells will survive. Thus, all living transgenic animals have XEN cells. In this scenario, the selection process is based on survival.

In some aspects, the genetically modified animals can be modified in a way that humanizes all or a portion of the animal. For example, only the endodermal pathway genes can be humanized or any gene or protein that contacts an organ of interest, such as the liver or pancreas, of the modified animal can be humanized. Examples of genes that can be humanized can be, but are not limited to, hepatic growth factor, fibroblast growth factor, human leukocyte antigen (HLA) genes, complement genes, immunoglobulin genes, or other genes involved in immune regulation. Humanizing a gene refers to swamping a gene of a host animal for a human gene sequence and wherein expression of the human sequence is driven by a promoter of the host animal. For example, the pig hepatic growth factor can be swapped for the cDNA of human hepatic growth factor in a pig and expression of human hepatic growth factor is driven by a pig promoter. Humanizing can be accomplished by genome editing using any of the techniques described herein or known in the art.

1. Genetic Modification Techniques

The disclosed genetically modified animals can be modified using any known technique in the art. These techniques can result in the removal of a gene, mutation of a gene, suppression of gene expression, or complete inactivation of a gene.

i. Transposons

In some embodiments, genetic modification is performed through the use of DNA transposons. Genetic modification of stem cells using DNA transposons is described, for example, in WO/2010/065550, which is incorporated by reference herein in its entirety. DNA transposons can be viewed as natural gene delivery vehicles that integrate into the host genome via a “cut-and-paste” mechanism. These mobile DNA elements encode a transposase flanked by inverted terminal repeats (ITRs) that contain the transposase binding sites necessary for transposition. Any gene of interest flanked by such ITRs can undergo transposition in the presence of the transposase supplied in trans. As noted, a “transposon” is a segment of DNA that can move (transpose) within the genome. A transposon may or may not encode the enzyme transposase, necessary to catalyze its relocation and/or duplication in the genome. Where a transposon does not code for its transposase enzyme, expression of said enzyme in trans may be required when carrying out the method of the invention in cells not expressing the relevant transposase itself. Furthermore, a transposon must contain sequences that are required for its mobilization, namely the terminal inverted repeats containing the binding sites for the transposase. The transposon may be derived from a bacterial or a eukaryotic transposon. Further, the transposon may be derived from a class I or class II transposon. Class II or DNA-mediated transposable elements are preferred for gene transfer applications, because transposition of these elements does not involve a reverse transcription step, which pertains in transposition of Class I or retro-elements and which can introduce undesired mutations into transgenes. For example, see Miller, A. D., RETROVIRUSES 843 (Cold Spring Harbor Laboratory Press, 1997), and Verma, L M. et al., Nature 389:239 (1997).

Transposons also can be harnessed as vehicles for introducing “tagged” genetic mutations into genomes, which makes such genomic sites of transposon integration/mutation easy to clone and defined at the DNA sequence level. This fact makes transposon-based technology especially attractive in cultures of germline stem cells derived from a variety of species. For example, the first mutagenesis screens in mammals have established that the Sleeping Beauty transposon system can generate a high number of random mutations in both mouse and rat germinal cells in vivo. Alternatively, where mutagenic events can first be selected and then used to produce experimental animal models, random mutagenesis would be more feasible in tissue culture.

Similarly, transposons can be hamessed as vehicles for introducing mutations into genomes. Specifically, genes may be inactivated by transposon insertion. For example, such genes are then “tagged” by the transposable element, which can be used for subsequent cloning of the mutated allele. In addition to gene inactivation, a transposon may also introduce a transgene of interest into the genome if contained between its ITRs. Moreover, to insert or knockin a DNA construct or gene of interest into an existing site of transposition, stem cell lines or animals produced with transposons are designed to contain recognition sequences (e.g., pLox sties) within the transposon that act as substrates for DNA recombinase enzymes (e.g., Cre-recombinase). This would allow a gene of interest flanked by compatible recombinase recognition sequences to be delivered into the cells or animals in trans with a recombinase to catalyze integration of the gene of interest into the genomic locus of the transposon. The transposon may carry as well the regulatory elements necessary for the expression of the transgene, allowing for successful expression of the gene. Examples of transposon systems that can transpose in vertebrates have recently became available, such as Sleeping Beauty, piggyBac, Tol2 or Frog Prince. Each transposon system can be combined with any gene trap mechanism (for example, enhancer, promoter, polyA, or slice acceptor gene traps) to generate the mutated gene, as discussed below. Sleeping Beauty (SB) and Frog Prince (FP) are Tcl transposons, whereas piggyBac (PB) was the founder of the PB transposon family and Tol2 is a hAT transposon family member. Both the Sleeping Beauty and the Frog Prince transposon are found in vertebrates as inactive copies, from which active transposon systems have been engineered. The Tol2 transposon also has been found in vertebrates as an active transposon. The piggyBac transposon was originally found as an active transposon in insects but was subsequently shown to have high levels of activity in vertebrates, too, as shown in Ding S et al, Cell 122:473(2005). Each of these elements has their own advantages; for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore mutagenize overlapping, but distinct sets of genes. Therefore, to achieve the best possible coverage of genes, the use of more than one element is particularly preferred. In addition to naturally occurring transposons, modified transposon systems such as those disclosed in European patent documents EP1594973, EP 1594971, and EP1594972 also may be employed. In some embodiments, the transposons used possess highly elevated transpositional activity. In some embodiments, the transposon is a eukaryotic transposon, such as the Sleeping Beauty transposon, the Frog Prince transposon, the piggyBac transposon, or the Tol2 transposon, as discussed above.

The use of gene-trap constructs for insertional mutagenesis in tissue culture, where trapped events can easily be selected for, is advantageous over the random mutagenesis in animals. Gene trap vectors report both the insertion of the transposon into an expressed gene, and have a mutagenic effect by truncating the transcript through imposed splicing. Cells selected for a particular gene trap event can be used for the generation of animal models lacking this specific genetic function.

When transposons are used in insertional mutagenesis screens, transposon vectors typically constitute four major classes of constructs, suitable for identifying mutated genes rapidly. These contain a reporter gene, which should be expressed depending on the genetic context of the integration. Specific gene traps include, but are not limited to: (1) enhancer traps, (2) promoter traps, (3) polyA traps, and (4) splice acceptor traps. In enhancer traps, the expression of the reporter requires the presence of a genomic cis-regulator to act on an attenuated promoter within the integrated construct. Promoter traps contain no promoter at all. These vectors are only expressed if they land in-frame in an exon or close downstream to a promoter of an expressed gene. In polyA traps, the marker gene lacks a polyA signal, but contains a splice donor (SD) site. Thus, when integrating into an intron, a fusion transcript can be synthesized comprising the marker and the downstream exons of the trapped gene. Slice acceptor gene traps (or exon traps) also lack promoters, but are equipped with a splice acceptor (SA) preceding the marker gene. Reporter activation occurs if the vector is integrated into an expressed gene, and splicing between the reporter and an upstream exon takes place. The splice acceptor gene trap and polyA gene trap cassettes can be combined. In that case, the marker of the polyA trap part is amended with a promoter so that the vector also can trap downstream exons, and both upstream and downstream fusion transcripts of the trapped gene can be obtained. The foregoing constructs also offer the possibility to visualize spatial and temporal expression pattems of the mutated genes by using, e.g., LacZ or fluorescent proteins as a marker gene.

In some embodiments, the present invention relates to a method based on the combination of transposon-mediated insertional mutagenesis with a tissue culture system, e.g. culture of EF cells or fetal fibroblast (FF) cells, which allows for the ready generation of in vitro EF or FF cell libraries carrying a large number of different insertion events. Compared to classical nuclear transfer technologies and in vivo mutagenesis, moreover, this method is less costly and less labor-intensive, and it allows for the selection of the appropriate insertion(s) before establishing the corresponding animal models. Additionally, using these cells or libraries allows for establishment of a broader variety of animal models.

Libraries of EF or FF cell lines can be generated by isolating and then pooling individual clonal lines with mutated genes. First, EF or FF cell lines are genetically modified with a DNA construct that harbors a selectable marker, such as a gene encoding resistance to G418. Then, due to stable integration of the DNA construct into different locations within the genome, a mixed population of genetically distinct EF or FF cell lines is selected using the selectable marker. By pooling these selected individual clonal lines with mutated genes, a library of mutant EF or FF cell lines is generated.

The phrase “selectable marker” is employed here to denote a protein that enables the separation of cells expressing the marker from those that lack or do not express it. The selectable marker may be a fluorescent marker, for instance. Expression of the marker by cells having successfully integrated the transposon allows the isolation of these cells using methods such as, for example, FACS (fluorescent activated cell sorting). Alternatively, expression of a selectable marker may confer an advantageous property to the cell that allows survival of only those cells carrying the gene. For example, the marker protein may allow for the selection of the cell by conferring an antibiotic resistance to the cell. Consequently, when cells are cultured in medium containing said antibiotic, only cell clones expressing the marker protein that mediates antibiotic resistance are capable of propagating. By way of illustration, a suitable marker protein may confer resistance to antibiotics such as ampicillin, kanamycin, chloramphenicol, tetracycline, hygromycin, neomycin or methotrexate. Further examples of antibiotics are penicillins: ampicillin HCl, ampicillin Na, amoxycillin Na, carbenicillin disodium, penicillin G, cephalosporins, cefotaxim Na, cefalexin HCl, vancomycin, cycloserine. Other examples include bacteriostatic inhibitors such as: chloramphenicol, erythromycin, lincomycin, spectinomycin sulfate, clindamycin HCl, chlortetracycline HCl. Additional examples are marker proteins that allow selection with bactericidal inhibitors such as those affecting protein synthesis irreversibly causing cell death, for example aminoglycosides such as gentamycin, hygromycin B, kanamycin, neomycin, streptomycin, G418, tobramycin. Aminoglycosides can be inactivated by enzvmes such as NPT 1 which phosphorylates 3′-OH present on kanamycin, thus inactivating this antibiotic. Some aminoglycoside modifying enzymes acetylate the compounds and block their entry in to the cell. Marker proteins that allow selection with nucleic acid metabolism inhibitors like rifampicin, mitomycin C, nalidixic acid, doxorubicin HCl, 5-flurouracil, 6-mercaptopurine, antimetabolites, miconazole, trimethoprim, methotrexate, metronidazole, sulfametoxazole are also examples for selectable markers.

In some embodiments, the present disclosure relates to methods of integrating an exogenous nucleic acid into the genome of at least one cell of an animal comprising administering directly to the cell: a) a transposon comprising the exogenous nucleic acid, wherein the exogenous nucleic acid is flanked by one or more inverted repeat sequences that are recognized by any of the aforementioned proteins and b) any one of the aforementioned proteins to excise the exogenous nucleic acid from a plasmid, episome, or transgene and integrate the exogenous nucleic acid into the genome. Methods of genetically modifying cells of an animal using transposon are described, for example, in WO/2012/074758, which is incorporated by reference herein in its entirety. In some embodiments, the protein of b) is administered as a nucleic acid encoding the protein. In some embodiments, the transposon and nucleic acid encoding the protein of b) are present on separate vectors. In some embodiments, the transposon and nucleic acid encoding the protein of b) are present on the same vector. When present on the same vector, the portion of the vector encoding the hyperactive transposase is located outside the portion carrying the inserted nucleic acid. In other words, the transposase encoding region is located external to the region flanked by the inverted repeats. Put another way, the tranposase encoding region is positioned to the left of the left terminal inverted repeat or to the right of the right terminal inverted repeat. In the aforementioned methods, the hyperactive transposase protein recognizes the inverted repeats that flank an inserted nucleic acid, such as a nucleic acid that is to be inserted into a target cell genome.

In some embodiments, the organism is a livestock animal. In some embodiments the livestock animal is selected from the group consisting of a sheep, a goat, a cow, a pig and a horse.

The elements of the PiggyBac transposase system are administered to the cell in a manner such that they are introduced into a target cell under conditions sufficient for excision of the inverted repeat flanked nucleic acid from the vector carrying the transposon and subsequent integration of the excised nucleic acid into the genome of the target cell. As the transposon is introduced into the cell “under conditions sufficient for excision and integration to occur.” the method can further include a step of ensuring that the requisite PiggyBac transposase activity is present in the target cell along with the introduced transposon.

Depending on the structure of the transposon vector itself, such as whether or not the vector includes a region encoding a product having PiggyBac transposase activity, the method can further include introducing a second vector into the target cell that encodes the requisite transposase activity, where this step also includes an in vivo administration step.

The amount of vector nucleic acid comprising the transposon element, and in many embodiments the amount of vector nucleic acid encoding the transposase, which is introduced into the cell is sufficient to provide for the desired excision and insertion of the transposon nucleic acid into the target cell genome. As such, the amount of vector nucleic acid introduced should provide for a sufficient amount of transposase activity and a sufficient copy number of the nucleic acid that is desired to be inserted into the target cell. The amount of vector nucleic acid that is introduced into the target cell varies depending on the efficiency of the particular introduction protocol that is employed.

The particular dosage of each component of the system that is administered to the cell varies depending on the nature of the transposon nucleic acid, e.g. the nature of the expression module and gene, the nature of the vector on which the component elements are present, the nature of the delivery vehicle and the like. Dosages can readily be determined empirically by those of skill in the art.

Once the vector DNA has entered the target cell in combination with the requisite transposase, the nucleic acid region of the vector that is flanked by inverted repeats, i.e. the vector nucleic acid positioned between the PiggyBac transposase-recognized inverted repeats, is excised from the vector via the provided transposase and inserted into the genome of the targeted cell. As such, introduction of the vector DNA into the target cell is followed by subsequent transposase mediated excision and insertion of the exogenous nucleic acid carried by the vector into the genome of the targeted cell.

The subject methods may be used to integrate nucleic acids of various sizes into the target cell genome. Generally, the size of DNA that is inserted into a target cell genome using the subject methods ranges from about 0.5 kb to 100.0 kb, usually from about 1.0 kb to about 60.0 kb, or from about 1.0 kb to about 10.0 kb.

The subject methods result in stable integration of the nucleic acid into the target cell genome. By stable integration is meant that the nucleic acid remains present in the target cell genome for more than a transient period of time, and is passed on a part of the chromosomal genetic material to the progeny of the target cell. The subject methods of stable integration of nucleic acids into the genome of a target cell find use in a variety of applications in which the stable integration of a nucleic acid into a target cell genome is desired. Applications in which the subject vectors and methods find use include, for example, research applications, polypeptide synthesis applications and therapeutic applications. The hyperactive transposase can be delivered as DNA. RNA, or protein.

In some embodiments, the present disclosure relates to a colony of transgenic animals each such transgenic animal comprising one or more exogenous nucleic acid sequences and one or two internal tandem repeat sequences of the a transposon. The present disclosure also relates to one or more progeny from an animal comprising the one more more exogenous nucleic acid sequences and one or more internal tandem repeat sequences of the transposons. The present disclosure also relates to a colony of transgenic animals each such transgenic animal comprising one or more exogenous nucleic acid sequences and one or two internal tandem repeat sequences of the a transposon described herein. The present disclosure also relates to one or more progeny from an animal comprising the one or more exogenous nucleic acid sequences and one or more internal tandem repeat sequences of the transposons described herein.

The hyperactive PiggyBac transposase system described herein can be used for germline mutagenesis in a vertebrate species. One method would entail the production of transgenic animals by, for example, pronuclear injection of newly fertilized oocytes.

Typically, two types of transgenes can be produced; one transgene provides expression of the transposase (a “driver” transgene) in germ cells (i.e., developing sperm or ova) and the other transgene (the “donor” transgene) comprises a transposon containing gene-disruptive sequences, such as a gene trap. The transposase may be directed to the germline via a ubiquitously active promoter, such as the ROSA26 (Gt(ROSA)26Sor), pPol2 (Polr2a), or CMV/beta-actin (CAG) promoters. Alternately, one may use a germline-restricted promoter, such as the spermatid-specific Protamine-1 (Prml) promoter, for mutagenesis exclusively in developing sperm. In another embodiment, the germline specific promoter is a female-specific promoter (e.g., a ZP3 promoter).

2. Nucleases

i. XTN Nucleases

Xanthomonas TAL nucleases, referred to as XTNs from the bacterium Xanthomonas, bind DNA sequences in a site-specific manner as a mechanism to regulate their genes. Methods of using XTN nuclease for genetic modification of stem cells are described, for example, in WO/2012/158986, which is incorporated by reference herein in its entirety. XTNs can be modified in order to specifically bind to sites within the genome of many organisms. XTNs may be used to introduce targeted double-stranded or single-stranded breaks in the DNA, which can lead to small deletions at the site of the break during the Non-Homologous End Joining (NHEJ) process, thereby producing gene knockouts in cells and organisms. XTNs can also generate breaks in the DNA which can increase the frequency of exogenous sequence introduction by homologous recombination, thereby enabling specific gene editing (e.g.—correction or mutation) or producing gene knock-ins in cells and organisms.

A central repeat domain containing multiple repeat units consisting of 33-35 amino acids determines nucleotide binding sites. Two essential adjacent amino acids known as repeat variable di-residue or RVDs are present in each repeat domain and separately specify a targeted base. The repeat domains and RVDs can be modified in order to target a gene or locus with high specificity (Mahfouz et a. (2011) PNAS 108, 6, 2623-2628). By fusing nuclease cleavage domains such as Fok1 to the XTNs, a nuclease is produced which is able to generate mutations in the genome of organisms in a site-specific manner. In one embodiment, XTNs are used to generate site specific mutations XEN cells, EF cells, zygotes or embryos.

XTN DNA binding specificity depends on the number and order of repeats in the DNA binding domain. Repeats are generally composed of 34-35 amino acids. Nucleotide binding specificity is determined by the 12 and 13 amino acids, called the repeat variable diresidue (RVD), within the DNA binding domain repeats. The RVDs bind to one or more nucleotides and the code has been deciphered using arbitrary RVDs as follows: asaparagine/isoleucine (NI)=A; histidine/aspartic acid (HD)=C; asparagine/glycine (NG)=T; asparagines/asparagines (NN)=A, G; asparaginesserine (NS)=A. C. G and T. Since the RVD binding code is deciphered, natural or codon-optimized versions of natural XTNs can be used as a scaffold to generate sequence specific DNA binding XTNs. The repeats and RVDs in the DNA binding domains of XTNs may be modified and synthesized to generate site specific DNA binding XTNs. The DNA cleavage domain of nucleases are fused into the XTN to produce a hybrid XTN which binds to a specific site on the DNA and produces mutations.

Genetic modification of SSCs using XTNs requires undifferentiated SSCs, transfection of the SSCs with XTNs and a selection marker, clonal selection of genetically modified SSCs, germline transmission of genetically modified SSCs, and germline transmission of recipient founders.

The methods used in the present invention are comprised of a combination of genetic introduction methods, site-specific genetic modification or mutagenesis mechanisms of stem cells, and generation of site-specific genetically modified organisms from the stem cells. For all genetic modification or mutagenesis mechanisms one or more introduction and delivery method may be employed. The invention may include but is not limited to the methods described below.

In some embodiments, the site-specific genetic modification is produced in a stem cell. e.g. a zygote, embryo, XEN cell or EF cell. These stem cells can proliferate as cultured cells and be genetically modified without affecting their ability to differentiate into other cell types, including germ line cells. Generating site-specific mutations in stem cells, which can then be used to produce a genetically modified organism, first involves the design and development of a protein such as a XTN whose DNA binding domain is engineered for a specific target site within the genome. A protein consisting of both a DNA binding domain and a cleavage or insertional mutagenesis domain is developed.

In one embodiment of the invention, a site-specific mutagenesis technology is expressed in stem cells or human cells generating site-specific mutations. The binding domains of the site-specific mutagenesis technologies are modified to bind a particular location in the genome. The site-specific mutagenesis technology may be introduced into stem cells via transfection using lipofetamine. A transfection mixture may be prepared by mixing transfectamine with the site specific mutagenesis technology XTNs. After harvesting undifferentiated stem cells, one may then add transfection mixture to the cell suspension, incubate, wash and plate the stem cells onto fresh EF feeder layers.

Screening for XTN mediated site specific modification such as knockout mutations via NHEJ or knockin mutations using homologous recombination (HR) is done by selection with co-transfected vectors. SSCs are co-transfected with a XTN and a selection marker vector such as a fluorescent marker or antibody resistance within a lipid-based transfection reagent, 1 ug total DNA is transfected with a ratio of 500 ng XTN to 500 ng selection vector. Clones are isolated and propagated to sufficient numbers to isolate DNA for screening and sequencing.

ii. Zinc Finger Nucleases

The nuclease agent employed in the various methods and compositions disclosed herein can further comprise a zinc-finger nuclease (ZFN). Methods of genetically modifying stem cells with ZFNs are described, for example, in WO2015200805, which is incorporated by reference herein in its entirety. In one embodiment, each monomer of the ZFN comprises 3 or more zinc finger-based DNA binding domains, wherein each zinc finger-based DNA binding domain binds to a 3 bp subsite. In other embodiments, the ZFN is a chimeric protein comprising a zinc finger-based DNA binding domain operably linked to an independent nuclease. In one embodiment, the independent endonuclease is a Fokl endonuclease. In one embodiment, the nuclease agent comprises a first ZFN and a second ZFN, wherein each of the first ZFN and the second ZFN is operably linked to a Fokl nuclease subunit, wherein the first and the second ZFN recognize two contiguous target DNA sequences in each strand of the target DNA sequence separated by about 5-7 bp spacer, and wherein the Fok nuclease subunits dimerize to create an active nuclease to make a double strand break. See, for example, US20060246567; US20080182332; US20020081614; US20030021776; WO/2002/057308A2; US20130123484; US20100291048; WO/2011/017293A2; and Gaj et al. (2013) Trends in Biotechnology, 31(7):397-405 each of which is herein incorporated by reference in its entirety.

iii. CRISPR/Cas

The nuclease agent employed in the various methods and compositions can also comprise a CRISPR/Cas system. Methods of genetically modifying stem cells with the CRISPR/Cas system are described, for example, in WO2015200805, which is incorporated by reference herein in its entirety. Such systems can employ a Cas9 nuclease, which in some instances, is codon-optimized for the desired cell type in which it is to be expressed. The system further employs a fused crRNA-tracrRNA construct that functions with the codon-optimized Cas9. This single RNA is often referred to as a guide RNA or gRNA. Within a gRNA, the crRNA portion is identified as the “target sequence’ for the givers recognition site and the tracrRNA is often referred to as the‘scaffold’. This system has been shown to function in a variety of eukaryotic and prokaryotic cells. Briefly, a short DNA fragment containing the target sequence is inserted into a guide RNA expression plasmid. The gRNA expression plasmid comprises the target sequence (in some embodiments around 20 nucleotides), a form of the tracrRNA sequence (the scaffold) as well as a suitable promoter that is active in the cell and necessary elements for proper processing in eukaryotic cells. Many of the systems rely on custom, complementary oligos that are annealed to form a double stranded DNA and then cloned into the gRNA expression plasmid. The gRNA expression cassette and the Cas9 expression cassette are then introduced into the cell. See, for example, Mali P et al. (2013) Science 2013 Feb. 15; 339 (6121):823-6; Jinek M et al. Science 2012 Aug. 17:337(6096):816-21; Hwang W Y et al. Nat Biotechnol 2013 March; 31(3):227-9; Jiang W et al. Nat Biotechnol 2013 March; 31(3):233-9; and, Cong L et al. Science 2013 Feb. 15:339(6121):819-23, each of which is herein incorporated by reference.

The methods and compositions disclosed herein can utilize Clustered Regularly Interspersed Short Palindromic Repeats (CRISPR)/CRISPR-associated (Cas) systems or components of such systems to modify a genome within a cell. CRISPR/Cas systems include transcripts and other elements involved in the expression of, or directing the activity of, Cas genes. A CRISPR/Cas system can be a type I, a type II, or a type III system. The methods and compositions disclosed herein employ CRISPR/Cas systems by utilizing CRISPR complexes (comprising a guide RNA (gRNA) complexed with a Cas protein) for site-directed cleavage of nucleic acids.

Some CRISPR/Cas systems used in the methods disclosed herein are non-naturally occurring. A “non-naturally occurring” system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together.

a. Cas RNA-Guided Endonucleases

Cas proteins generally comprise at least one RNA recognition or binding domain. Such domains can interact with guide RNAs (gRNAs, described in more detail below). Cas proteins can also comprise nuclease domains (e.g., DNase or RNase domains), DNA binding domains, helicase domains, protein-protein interaction domains, dimerization domains, and other domains. A nuclease domain possesses catalytic activity for nucleic acid cleavage. Cleavage includes the breakage of the covalent bonds of a nucleic acid molecule. Cleavage can produce blunt ends or staggered ends, and it can be single-stranded or double-stranded.

Examples of Cas proteins include Cas1, Cas1B, Cas2, Cas3, Cas4, Cas5. Cas5e (CasD), Cas6, Cas6e, Cas6f, Cas7, Cas8a1, Cas8a2, Cas8b, Cas8c, Cas9 (Csnl or Csx12), CaslO, CaslOd, CasF, CasG, CasH, Csy1, Csy2, Csy3, Csel (CasA), Cse2 (CasB), Cse3 (CasE), Cse4 (CasC), Csc1, Csc2, Csa5, Csn2, Csm2, Csm3, Csm4, Csm5. Csm6, Cmrl, Cmr3, Cmr4, Cmr5, Cmr6, Csb1, Csb2, Csb3, Csxl7, Csx14, CsxlO, Csx16, CsaX, Csx3, Csxl, Csxl5, Csfl, Csf2, Csf3, Csf4, and Cul966, and homologs or modified versions thereof.

Cas proteins can be from a type II CRISPR/Cas system. For example, the Cas protein can be a Cas9 protein or be derived from a Cas9 protein. Cas9 proteins typically share four key motifs with a conserved architecture. Motifs 1, 2, and 4 are RuvC-like motifs, and motif 3 is an HNH motif. The Cas9 protein can be from, for example, Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile. Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acarvochloris marina. Additional examples of the Cas9 family members are described in WO 2014/131833, herein incorporated by reference in its entirety. Cas9 protein from S. pyogenes or derived therefrom is a preferred enzyme. Cas9 protein from S. pyogenes is assigned SwissProt accession number Q99ZW2.

Cas proteins can be wild type proteins (i.e., those that occur in nature), modified Cas proteins (i.e., Cas protein variants), or fragments of wild type or modified Cas proteins. Cas proteins can also be active variants or fragments of wild type or modified Cas proteins. Active variants or fragments can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

Cas proteins can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

Some Cas proteins comprise at least two nuclease domains, such as DNase domains. For example, a Cas9 protein can comprise a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337:816-821, hereby incorporated by reference in its entirety.

One or both of the nuclease domains can be deleted or mutated so that they are no longer functional or have reduced nuclease activity. If one of the nuclease domains is deleted or mutated, the resulting Cas protein (e.g., Cas9) can be referred to as a nickase and can generate a single-strand break at a CRISPR RNA recognition sequence within a double-stranded DNA but not a double-strand break (i.e., it can cleave the complementary strand or the non-complementary strand, but not both). If both of the nuclease domains are deleted or mutated, the resulting Cas protein (e.g., Cas9) will have a reduced ability to cleave both strands of a double-stranded DNA. An example of a mutation that converts Cas9 into a nickase is a DIOA (aspartate to alanine at position 10 of Cas9) mutation in the RuvC domain of Cas9 from S. pyogenes. Likewise, H939A (histidine to alanine at amino acid position 839) or H840A (histidine to alanine at amino acid position 840) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Research 39:9275-9282 and WO 2013/141680, each of which is herein incorporated by reference in its entirety. Such mutations can be generated using methods such as site-directed mutagenesis, PCR-mediated mutagenesis, or total gene synthesis. Examples of other mutations creating nickases can be found, for example, in WO/2013/176772A1 and WO/2013/142578A1, each of which is herein incorporated by reference.

Cas proteins can also be fusion proteins. For example, a Cas protein can be fused to a cleavage domain, an epigenetic modification domain, a transcriptional activation domain, or a transcriptional repressor domain. See WO 2014/089290, incorporated herein by reference in its entirety. Cas proteins can also be fused to a heterologous polypeptide providing increased or decreased stability. The fused domain or heterologous polypeptide can be located at the N-terminus, the C-terminus, or intemally within the Cas protein.

A Cas protein can be fused to a heterologous polypeptide that provides for subcellular localization. Such heterologous peptides include, for example, a nuclear localization signal (NLS) such as the SV40 NLS for targeting to the nucleus, a mitochondrial localization signal for targeting to the mitochondria, an ER retention signal, and the like. See, e.g., Lange et al. (2007)/. Biol. Chem. 282:5101-5105. Such subcellular localization signals can be located at the N-terminus, the C-terminus, or anywhere within the Cas protein. An NLS can comprise a stretch of basic amino acids, and can be a monopartite sequence or a bipartite sequence.

Cas proteins can also be linked to a cell-penetrating domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, for example, WO 2014/089290, herein incorporated by reference in its entirety. The cell-penetrating domain can be located at the N-terminus, the C-terminus, or anywhere within the Cas protein.

Cas proteins can also comprise a heterologous polypeptide for ease of tracking or purification, such as a fluorescent protein, a purification tag, or an epitope tag. Examples of fluorescent proteins include green fluorescent proteins (e.g., GFP. GFP-2, tagGFP, turboGFP, eGFP, Emerald, Azami Green, Monomeric Azami Green. CopGFP, AceGFP, ZsGreenl), yellow fluorescent proteins (e.g., YFP, eYFP, Citrine, Venus, YPet, PhiYFP, ZsYellowl), blue fluorescent proteins (e.g. eBFP, eBFP2, Azurite, mKalamal, GFPuv, Sapphire, T-sapphire), cyan fluorescent proteins (e.g. eCFP. Cerulean, CyPet, AmCyanl, Midoriishi-Cyan), red fluorescent proteins (mKate, mKate2, mPlum, DsRed monomer, mCherrv, mRFPl, DsRed-Express, DsRed2, DsRed-Monomer, HcRed-Tandem. HcRedl, AsRed2, eqFP611, mRaspber, mStrawberry, Jred), orange fluorescent proteins (mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcVS, AU1, AU5, E, ECS, E2, FLAG, hemagglutinin (HA), nus, Softag 1, Softag 3, Strep, SBP, Glu-Glu, HSV, KT3, S, ST, T7, V5, VSV-G, histidine (His), biotin carboxyl carrier protein (BCCP), and calmodulin.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. In some embodiments, the Cas protein is any amino acid and nucleic acid sequences associated with the Accession Numbers below as of Apr. 17, 2019, all such sequences are incorporated by reference in their entireties. Any mutants or variants that are at least 70, 75, 80, 85, 90, 95, 96, 97, 98, 99% homologous to the encoded nucleic acids or acids set forth in the Accession Numbers below are also incorporated by reference in their entireties.

NC . . . 014644. 1 NC . . . 002967. 9, NC . . . 007929. 1 NC . . . 000913. 3 NC . . . 004547. 2, NC. 0.009380. 1 NC . . . 011661 1; NC . . . 010175. 1 NC . . . 010175. 1 NC . . . 010175. 1 NC . . . 003413 . . . 1 NC. 0.000917. 1 NC. 0.002939, 5 NC . . . 018227 2; NC . . . 004829. 2, NC. 0.021921. 1 NC . . . 014160. 1 NC. 0.011766 . . . 1 NC . . . 007681. 1 NC . . . 021592. 1 NC . . . 021592 1; NC . . . 021169. 1 NC . . . 020517. 1 NC. 0.018656. 1 NC . . . 018015 . . . 1 NC . . . 018015. 1 NC . . . 017946. 1 NC . . . 017576 1; NC . . . 017576. 1 NC. 0.015865. 1 NC . . . 015865. 1 NC . . . 015680 . . . 1 NC . . . 015680. 1 NC . . . 015474. 1 NC . . . 015435 1:NC. 0.013790. 1 NC . . . 013790. 1 NC . . . 012883. 1 NC . . . 012470 . . . 1 NC . . . 016051. 1 NC . . . 010610. 1 NC. 0.009515 1:NC . . . 008942. 1 NC . . . 007181. 1 NC . . . 007181. 1 NC . . . 006624 . . . 1 NC . . . 006448. 1 NC. 0.002935. 2 NC . . . 002935 2; NC. 0.002950.2, NC. 0.002950. 2, NC . . . 002663. 1 NC . . . 002663. 0.1 NC . . . 004557. 1 NC . . . 004557. 1 NC . . . 019943 1; NC . . . 019943. 1 NC . . . 019943. 1 NC . . . 017459. 1 NC . . . 017459 . . . 1 NC . . . 015518. 1 NC . . . 015460. 1 NC . . . 015416 1:

Nucleic acids encoding Cas proteins can be stably integrated in the genome of the cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g. a Cas gene) and which can transfer such a nucleic acid sequence of interest to a target cell.

b. Guide RNAs (gRNAs)

A “guide RNA” or “gRNA” includes an RNA molecule that binds to a Cas protein and targets the Cas protein to a specific location within a target DNA. Guide RNAs can comprise two segments: a “DNA-targeting segment” and a “protein-binding segment.” “Segment” includes a segment, section, or region of a molecule, such as a contiguous stretch of nucleotides in an RNA. Some gRNAs comprise two separate RNA molecules: an “activator-RNA” and a “targeter-RNA.” Other gRNAs are a single RNA molecule (single RNA polynucleotide), which can also be called a “single-molecule gRNA,” a “single-guide RNA,” or an “sgRNA.” See, e.g., WO/2013/176772A1, WO/2014/065596A1, WO/2014/089290A1, WO/2014/093622A2, WO/2014/099750A2, WO/2013142578A1, and WO 2014/131833A1, each of which is herein incorporated by reference. The terms “guide RNA” and “gRNA” include both double-molecule gRNAs and single-molecule gRNAs.

An exemplary two-molecule gRNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA” or “scaffold”) molecule. A crRNA comprises both the DNA-targeting segment (single-stranded) of the gRNA and a stretch of nucleotides that forms one half of the dsRNA duplex of the protein-binding segment of the gRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA.

The crRNA and the corresponding tracrRNA hybridize to form a gRNA. The crRNA additionally provides the single-stranded DNA-targeting segment that hybridizes to a CRISPR RNA recognition sequence. If used for modification within a cell, the exact sequence of a given crRNA or tracrRNA molecule can be designed to be specific to the species in which the RNA molecules will be used. See, for example, Mali et al. (2013) Science 339:823-826: Jinek et al. (2012) Science 337:816-821; Hwang et al. (2013) Nat. Biotechnol. 31:227-229; Jiang et al. (2013) Nat. Biotechnol. 31:233-239; and Cong et al. (2013) Science 339:819-823, each of which is herein incorporated by reference.

The DNA-targeting segment (crRNA) of a given gRNA comprises a nucleotide sequence that is complementary to a sequence in a target DNA. The DNA-targeting segment of a gRNA interacts with a target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the Cas9 system and organism but often contain a targeting segment of between 21 to 72 nucleotides length, flanked by two direct repeats (DR) of a length of between 21 to 46 nucleotides (see. e.g., WO2014/131833). In the case of 5″ pyogenes, the DRs are 36 nucleotides long and the targeting segment is 30 nucleotides long. The 3′ located DR is complementary to and hybridizes with the corresponding tracrRNA, which in turn binds to the Cas9 protein.

The DNA-targeting segment can have a length of from about 12 nucleotides to about 100 nucleotides. For example, the DNA-targeting segment can have a length of from about 12 nucleotides (nt) to about 80 nt, from about 12 nt to about 50 nt, from about 12 nt to about 40 nt, from about 12 nt to about 30 nt from about 12 nt to about 25 nt, from about 12 nt to about 20 nt, or from about 12 nt to about 19 nt. Alternatively, the DNA-targeting segment can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt.

The nucleotide sequence of the DNA-targeting segment that is complementary to a nucleotide sequence (CRISPR RNA recognition sequence) of the target DNA can have a length at least about 12 nt. For example, the DNA-targeting sequence (i.e., the sequence within the DNA-targeting segment that is complementary to a CRISPR RNA recognition sequence within the target DNA) can have a length at least about 12 nt, at least about 15 nt, at least about 18 nt, at least about 19 nt, at least about 20 nt, at least about 25 nt, at least about 30 nt, at least about 35 nt, or at least about 40 nt. Alternatively, the DNA-targeting sequence can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the DNA-targeting sequence can have a length of at about 20 nt.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise or consist of all or a portion of a wild-type tracrRNA sequence (e.g., about or more than about 20, 26, 32, 45, 48, 54, 63, 67. 85, or more nucleotides of a wild-type tracrRNA sequence). Examples of wild-type tracrRNA sequences from S. pyogenes include 171-nucleotide, 89-nucleotide. 75-nucleotide, and 65-nucleotide versions. See, for example, Deltcheva et al. (2011) Nature 471:602-607: WO 2014/093661, each of which is incorporated herein by reference in their entirety. Examples of tracrRNAs within single-guide RNAs (sgRNAs) include the tracrRNA segments found within +48, +54, +67, and +85 versions of sgRNAs, where “+n” indicates that up to the +n nucleotide of wild-type tracrRNA is included in the sgRNA. See U.S. Pat. No. 8,697,359, incorporated herein by reference in its entirety.

The percent complementarity between the DNA-targeting sequence and the CRISPR RNA recognition sequence within the target DNA can be at least 60% (e.g., at least 65%, at least 70%, at least 75%, at least 80%, at least 85%, at least 90%, at least 95%, at least 97%, at least 98%, at least 99%, or 100%). The percent complementarity between the DNA-targeting sequence and the CRISPR RNA recognition sequence within the target DNA can be at least 60% over about 20 contiguous nucleotides. As an example, the percent complementarity between the DNA-targeting sequence and the CRISPR RNA recognition sequence within the target DNA is 100% over the 14 contiguous nucleotides at the 5′ end of the CRISPR RNA recognition sequence within the complementary strand of the target DNA and as low as 0% over the remainder. In such a case, the DNA-targeting sequence can be considered to be 14 nucleotides in length. As another example, the percent complementarity between the DNA-targeting sequence and the CRISPR RNA recognition sequence within the target DNA is 100% over the seven contiguous nucleotides at the 5′ end of the CRISPR RNA recognition sequence within the complementary strand of the target DNA and as low as 0 % over the remainder. In such a case, the DNA-targeting sequence can be considered to be 7 nucleotides in length.

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Guide RNAs can include modifications or sequences that provide for additional desirable features (e.g., modified or regulated stability; subcellular targeting; tracking with a fluorescent label: a binding site for a protein or protein complex; and the like). Examples of such modifications include, for example, a 5′ cap (e.g., a 7-methylguanylate cap (m7G)): a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like) and combinations thereof.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the RNA. The DNA encoding the gRNA can encode a single RNA molecule (sgRNA) or separate RNA molecules (e.g., separate crRNA and tracrRNA). In the latter case, the DNA encoding the gRNA can be provided as separate DNA molecules encoding the crRNA and tracrRNA, respectively. DNAs encoding gRNAs can be stably integrated in the genome of the cell and operably linked to a promoter active in the cell. Alternatively, DNAs encoding gRNAs can be operably linked to a promoter in an expression construct.

Alternatively, gRNAs can be prepared by various other methods. For example, gRNAs can be prepared by in vitro transcription using, for example, T7 RNA polymerase (see, for example, WO 2014/089290 and WO 2014/0655%). Guide RNAs can also be a synthetically produced molecule prepared by chemical synthesis.

c. CRISPR RNA Recognition Sequences

The term “CRISPR RNA recognition sequence” includes nucleic acid sequences present in a target DNA to which a DNA-targeting segment of a gRNA will bind, provided sufficient conditions for binding exist. For example, CRISPR RNA recognition sequences include sequences to which a guide RNA is designed to have complementarity, where hybridization between a CRISPR RNA recognition sequence and a DNA targeting sequence promotes the formation of a CRISPR complex. Full complementarity is not necessarily required, provided there is sufficient complementarity to cause hybridization and promote formation of a CRISPR complex. CRISPR RNA recognition sequences also include cleavage sites for Cas proteins, described in more detail below. A CRISPR RNA recognition sequence can comprise any polynucleotide, which can be located, for example, in the nucleus or cytoplasm of a cell or within an organelle of a cell.

The CRISPR RNA recognition sequence within a target DNA can be targeted by (i.e., be bound by, or hybridize with, or be complementary to) a Cas protein or a gRNA. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual. 3rd Ed. (Sambrook et al., Harbor Laboratory Press 2001)). The strand of the target DNA that is complementary to and hybridizes with the Cas protein or gRNA can be called the “complementary strand,” and the strand of the target DNA that is complementary to the “complementary strand” (and is therefore not complementary to the Cas protein or gRNA) can be called “noncomplementary strand” or “template strand.”

The Cas protein can cleave the nucleic acid at a site within or outside of the nucleic acid sequence present in the target DNA to which the DNA-targeting segment of a gRNA will bind. The “cleavage site” includes the position of a nucleic acid at which a Cas protein produces a single-strand break or a double-strand break. For example, formation of a CRISPR complex (comprising a gRNA hybridized to a CRISPR RNA recognition sequence and complexed with a Cas protein) can result in cleavage of one or both strands in or near (e.g., within 1, 2, 3, 4, 5, 6, 7, 8, 9, 10, 20, 50, or more base pairs from) the nucleic acid sequence present in a target DNA to which a DNA-targeting segment of a gRNA will bind. If the cleavage site is outside of the nucleic acid sequence to which the DNA-targeting segment of the gRNA will bind, the cleavage site is still considered to be within the “CRISPR RNA recognition sequence.” The cleavage site can be on only one strand or on both strands of a nucleic acid. Cleavage sites can be at the same position on both strands of the nucleic acid (producing blunt ends) or can be at different sites on each strand (producing staggered ends). Staggered ends can be produced, for example, by using two Cas proteins, each of which produces a single-strand break at a different cleavage site on each strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the CRISPR RNA recognition sequence of the nickase on the first strand is separated from the CRISPR RNA recognition sequence of the nickase on the second strand by at least 2, 3.4. 5, 6, 7, 8, 9, 10, 15, 20, 25, 30, 40, 50. 75, 100, 250, 500, or 1,000 base pairs.

Site-specific cleavage of target DNA by Cas9 can occur at locations determined by both (i) base-pairing complementarity between the gRNA and the target DNA and (ii) a short motif, called the protospacer adjacent motif (PAM), in the target DNA. The PAM can flank the CRISPR RNA recognition sequence. Optionally, the CRISPR RNA recognition sequence can be flanked by the PAM. For example, the cleavage site of Cas9 can be about 1 to about 10 or about 2 to about 5 base pairs (e.g., 3 base pairs) upstream or downstream of the PAM sequence. In some cases (e.g., when Cas9 from S. pyogenes or a closely related Cas9 is used), the PAM sequence of the non-complementary strand can be 5′-NiGG-3′, where Niis any DNA nucleotide and is immediately 3′ of the CRISPR RNA recognition sequence of the non-complementary strand of the target DNA. As such, the PAM sequence of the complementary strand would be 5′-CC N2-3′, where N2 is any DNA nucleotide and is immediately 5′ of the CRISPR RNA recognition sequence of the complementary strand of the target DNA. In some such cases, Ni and N2 can be complementary and the Ni-N2 base pair can be any base pair (e.g., Ni=C and N2=G; Ni=G and N2=C; Ni=A and N2=T, Ni=T, and N2=A).

Examples of CRISPR RNA recognition sequences include a DNA sequence complementary to the DNA-targeting segment of a gRNA, or such a DNA sequence in addition to a PAM sequence. For example, the target motif can be a 20-nucleotide DNA sequence immediately preceding an NGG motif recognized by a Cas protein (see, for example, WO 2014/165825). The guanine at the 5′ end can facilitate transcription by RNA polymerase in cells. Other examples of CRISPR RNA recognition sequences can include two guanine nucleotides at the 5′ end (e.g., GGN20NGG; SEQ ID NO: 9) to facilitate efficient transcription by T7 polymerase in vitro. See, for example, WO 2014/065596.

The CRISPR RNA recognition sequence can be any nucleic acid sequence endogenous or exogenous to a cell. The CRISPR RNA recognition sequence can be a sequence coding a gene product (e.g., a protein) or a non-coding sequence (e.g., a regulatory sequence) or can include both.

In one embodiment, the target sequence is immediately flanked by a Protospacer Adjacent Motif (PAM) sequence. In one embodiment, the gRNA comprises a third nucleic acid sequence encoding a Clustered Regularly Interspaced Short Palindromic Repeats (CRISPR) RNA (crRNA) and a trans-activating CRISPR RNA (tracrRNA). In another embodiment, the genome of the pluripotent cell comprises a target DNA region complementary to the target sequence. In some such methods, the Cas protein is Cas9.

Active variants and fragments of nuclease agents (i.e. an engineered nuclease agent) may also be used. Such active variants can comprise at least 65%. 70%, 75%, 80%, 85%, 90%, 91%, 92%, 93%, 94%, 95%. 96%, 97%. 98%, 99% or more sequence identity to the native nuclease agent, wherein the active variants retain the ability to cut at a desired recognition site and hence retain nick or double-strand-break-inducing activity. For example, any of the nuclease agents described herein can be modified from a native endonuclease sequence and designed to recognize and induce a nick or double-strand break at a recognition site that was not recognized by the native nuclease agent. Thus, in some embodiments, the engineered nuclease has a specificity to induce a nick or double-strand break at a recognition site that is different from the corresponding native nuclease agent recognition site. Assays for nick or double-strand-break-inducing activity are known and generally measure the overall activity and specificity of the endonuclease on DNA substrates containing the recognition site.

The nuclease agent may be introduced into the cell by any means known in the art. The polypeptide encoding the nuclease agent may be directly introduced into the cell. Alternatively, a polynucleotide encoding the nuclease agent can be introduced into the cell. When a polynucleotide encoding the nuclease agent is introduced into the cell, the nuclease agent can be transiently, conditionally or constitutive expressed within the cell. Thus, the polynucleotide encoding the nuclease agent can be contained in an expression cassette and be operably linked to a conditional promoter, an inducible promoter, a constitutive promoter, or a tissue-specific promoter. Alternatively, the nuclease agent is introduced into the cell as an mRNA encoding a nuclease agent.

In specific embodiments, the polynucleotide encoding the nuclease agent is stably integrated in the genome of the cell and operably linked to a promoter active in the cell. In other embodiments, the polynucleotide encoding the nuclease agent is in the same targeting vector comprising the insert polynucleotide, while in other instances the polynucleotide encoding the nuclease agent is in a vector or a plasmid that is separate from the targeting vector comprising the insert polynucleotide.

When the nuclease agent is provided to the cell through the introduction of a polynucleotide encoding the nuclease agent, such a polynucleotide encoding a nuclease agent can be modified to substitute codons having a higher frequency of usage in the cell of interest, as compared to the naturally occurring polynucleotide sequence encoding the nuclease agent. For example the polynucleotide encoding the nuclease agent can be modified to substitute codons having a higher frequency of usage in a given eukaryotic cell of interest, as compared to the naturally occurring polynucleotide sequence.

### E. Chimeric Animals

Disclosed are chimeric animals. In some aspects, the animal can be a mammal. For example, the mammal can be, but is not limited to, a human, a non-human mammal, a primate, a sheep, a goat, a cow, a pig or a horse. The disclosure relates to a chimeric animal derived from an XEN cell and an embryo disclosed herein. In some embodiments, the chimeric animal is an animal comprising endodermal tissue with one or more modifications disclosed herein.

In some aspects, the chimeric animals can be chimeric throughout their entire body. In some aspects, the chimeric animals can be chimeric only in respect to certain organs, such as the liver and pancreas. For example, the cells within a specific organ are chimeric or the entire organ are chimeric. In some embodiments, entire embryonic-derived tissues are chimeric, such as those tissues derived from mesodermal or endodermal lineages.

In some aspects, chimeric animals can comprise XEN cells from an animal other than itself. In some aspects, the chimeric animals comprise cells that originated from XEN cells from an animal other than itself. For example, chimeric animals can comprise a liver, and thus, liver cells, that originated from XEN cells from an animal other than itself. In some aspects, chimeric animals can comprise cells or organs made from those cells, wherein those cells or organs are derived from the XEN cells transplanted in the animal to form the chimera.

### F. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for making chimeric animals, the kit comprising XEN cells. The kits also can contain gRNAs for making transgenic animals.

### EXAMPLES

### A. Example 1

Pluripotent stem cells (PSCs) like embryonic stem cells (ESC) or induced pluripotent stem cells (iPSCs) give rise to all the germ layers in the body. PSCs have long been considered and utilized for deriving organ-specific cells or miniature organs (organoids). Additionally, rodent PSCs vhen introduced into pre-implantation stage blastocyst embryos (also called blastocyst complementation), or specific conceptus (fetal or conceptus complementation) have contributed to cell type to form chimeras. However, these approaches are fraught with challenges, including the ability of the PSCs to contributing to extensive chimerism, including the ability to contribute to gonads, neurons, and other organs. When such PSCs from humans are used for blastocyst complementation in pigs, the risk of extensive human-pig chimerism is an unwarranted and unfavorable outcome. Hence, the use of cells that have limited developmental potential, specifically to the endodermal lineage when liver or pancreas need to be generated will be beneficial. This disclosure describes for the first time the use of endodermal precursors derived from pig embryos called extraembryonic endodermal cells (XEN cells from here after) that show extensive chimerism potential, with their contribution exclusively limited to the endodermal lineage. Potential exists for XEN cells from human origin to similarly contribute to 1) chimeras readily; and 2) contribute to endodermal cells and organs exclusively, in an organogenesis deficient pig model.

1. XEN Cells Effectively Contribute to Chimeras in a Pig Model

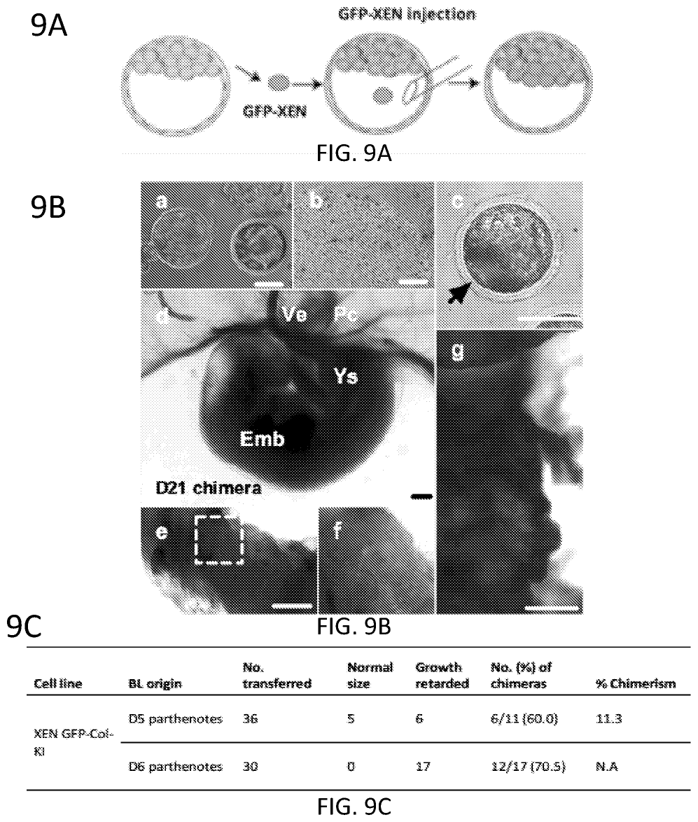

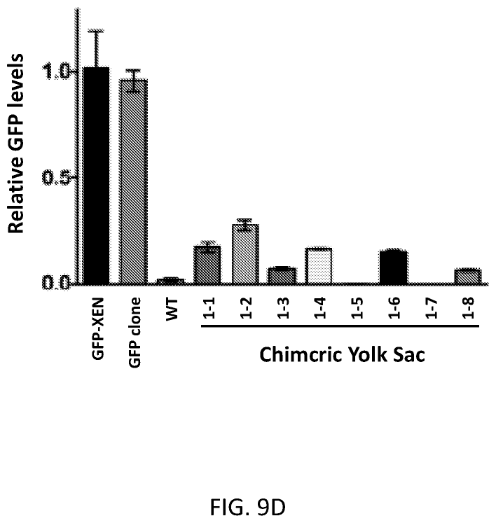

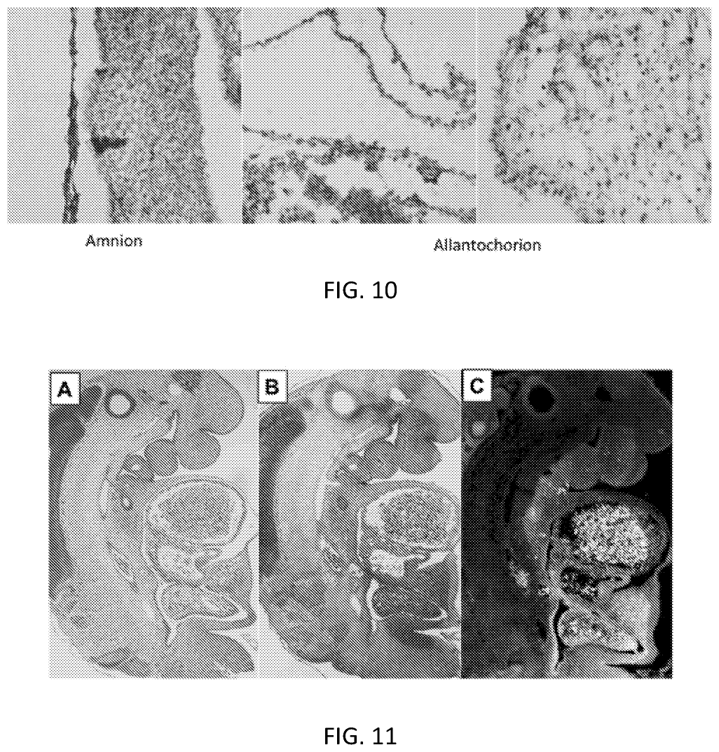

Using XEN cells that were described in International Application number PCT/US18/28041, hereby incorporated herein, it has been shown that introduction of the XEN cells that constitutively and stably express EGFP into parthenote-derived pig embryos have readily contributed to chimeras (FIG. 9), with the chimerism limited to extra-embyonic lineages (FIG. 10) and endodermal derivatives in the fetus (FIG. 11).

2. XEN Cells Effectively and Exclusively Contribute to Endodermal Derived Organs in Pig Chimeras

Staining of the cross section of fetuses derived from day 21 of pregnancy with endodermal lineage markers (GATA6 and SALL4) and antibody against GFP (anti-GFP), have tracked the contribution of the GFP+ve injected XEN cells to GATA6 and SALL4 derivatives in the fetus. This unique ability to contribute to endodermal but not other lineages (for e.g., mesodermal or neurectodermal derivatives) make it an ideal cell type for future human-pig organogenesis.

3. Potential for Pig-Human Chimeras

The establishment of human cell types in pig models can provide on-demand solution and source for human cells that could be used in regenerative medicine applications, such as cell therapy, ex vivo cell-based tools such as organoids, tissue-on-chip, and when an organ like liver is established, can be used for whole body toxicology and pharmacokinetic applications. Patient-specific iPS derived XEN cells can also be used to generate patient specific organs for autologous transplantation. While the applications are numerous, and long-term promise for on-demand organ generation exists, the establishment of XEN cells from non-rodent model (pig) and their contribution extensively to chimeras in a pig model offer for the first time the ability to generate targeted endodermal lineages and solid organs in a pig model.

### B. Example 2

1. Introduction

In mammals, delamination of primitive endoderm (PrE) from the inner cell mass (ICM) in the late blastocyst-stage embryo marks the second fate specification event (the first being the separation of trophectoderm (TE) from the ICM). The PrE differentiate into visceral endoderm (VE) and parietal endoderm (PE) that line ICM and TE, respectively. Together, the VE and PE generate yolk sac, the first placental membrane. The yolk sac serves as the main placenta in rodents until mid-gestation (d11.5), and performs several important functions including providing nutritional support, gas exchange, hematopoiesis, and patterning cues to the developing embryo. However, in non-rodent species including pig and humans, the yolk sac is short-lived. Regardless, in all species the PrE does not contribute to the embryonic endoderm, which emerges later following gastrulation4.

In culture, three types of stem cells can be established from the mouse embryo: embryonic stem cells (ESC) from the EPI, trophoblast stem cells (TSC) from TE, and XEN cells from PrE, which contribute to embryo proper, the placenta, and the yolk sac, respectively. The XEN cells can also be induced from ESC by overexpression of PrE-specific genes, Gata-4, 66, 7, or Sox178, or by treatment with growth factors9. More recently, naïve extraembryonic endodermal cells (nEnd) resembling the blastocyst-stage PrE-precursors have been developed from the authentic mouse ESC. In rat, XEN cells established from blastocysts have different culture requirements and gene expression profiles compared to mouse XEN cells. While, mouse XEN cells mainly contribute to the PE in chimeras, the rat XEN cells contribute to the VE. It is unclear whether XEN cells from non-rodent animals (human and pig) have potency similar to mouse or rat. In this regard, the pig model can prove to be uniquely valuable in bridging the translational gap between rodents and humans.

Authentic ESC from pigs (p) have yet to be generated even after three decades of extensive investigation. The major reason for difficulties in the derivation of pESC is the instability of the pluripotent state. Even though derivation of pESC from EPI cells has proven to be difficult, extraembryonic (ExE) cells within the early blastocyst outgrowths grow rapidly and outnumber the EPI cells, which can often be misinterpreted as epiblast cells. There are several reports describing pig EPI-like cells with properties similar to human ESC. However, these observations are purely conjectural, only fulfilling minimal criteria of pluripotency, and lacking the deterministic in vivo demonstration of pluripotency. Besides ESC, attempts to establish TSC and XEN cells from pig or other domestic animals has received little attention, and efforts to explore their potential is non-existent.

Described herein is the establishment of XEN cells from the PrE of the pig blastocysts. To-date these pXEN cells represent the only well characterized blastocyst-derived stem cell lines that can be readily and reproducibly established under current culture conditions. The pXEN cells are stable in culture, undergo self-renewal for extended periods of time, and contribute predominantly to yolk sac and at a minor level to embryonic endoderm (gut) in chimeras, and can serve as nuclear donors to generate live offspring.

2. Results

i. In Vitro Derivation and Expansion of Primary PrE Outgrowths

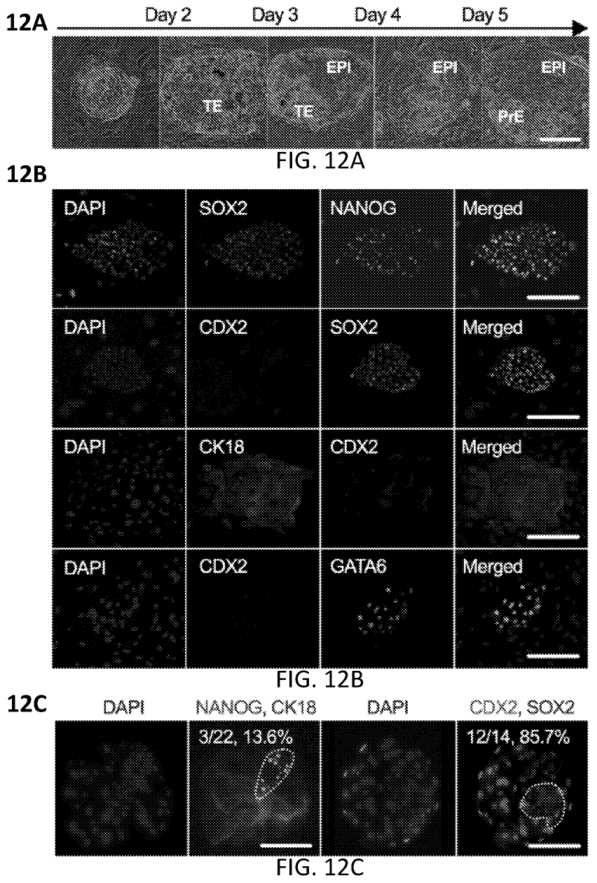

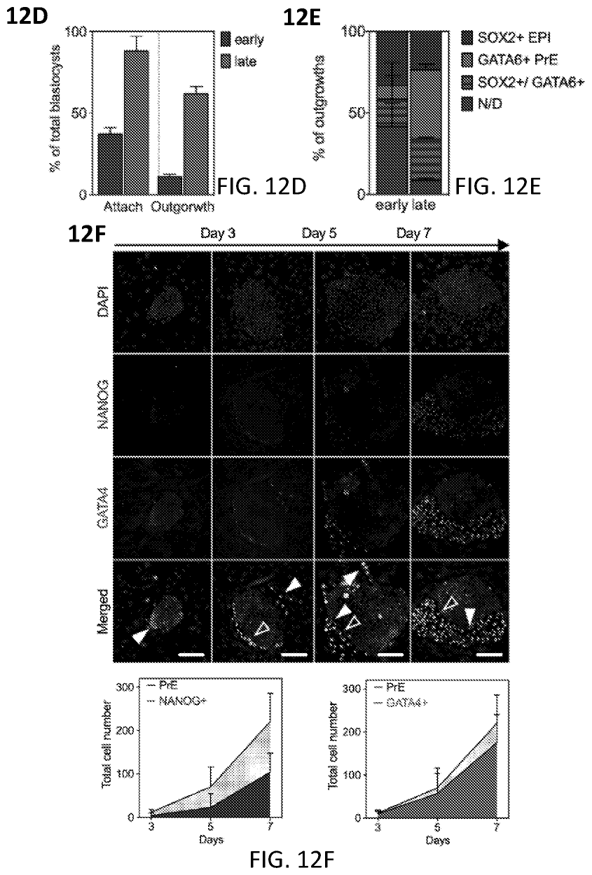

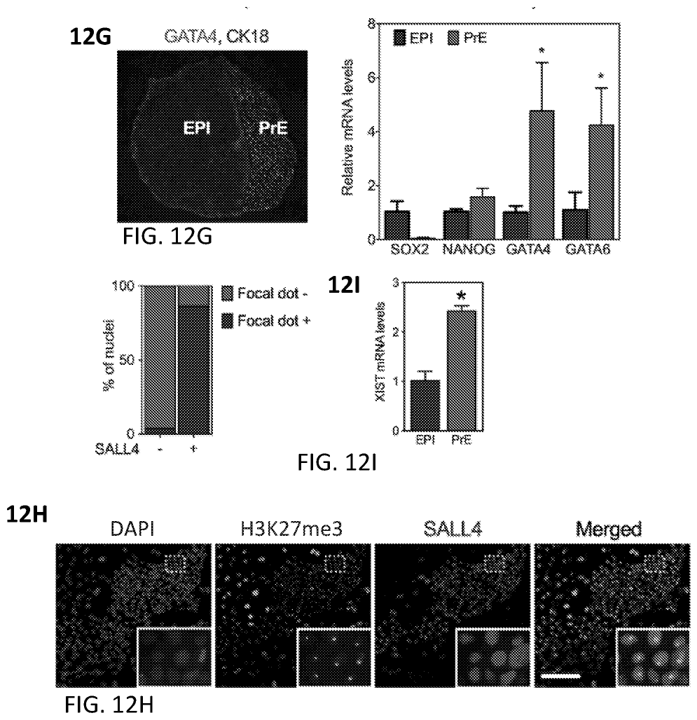

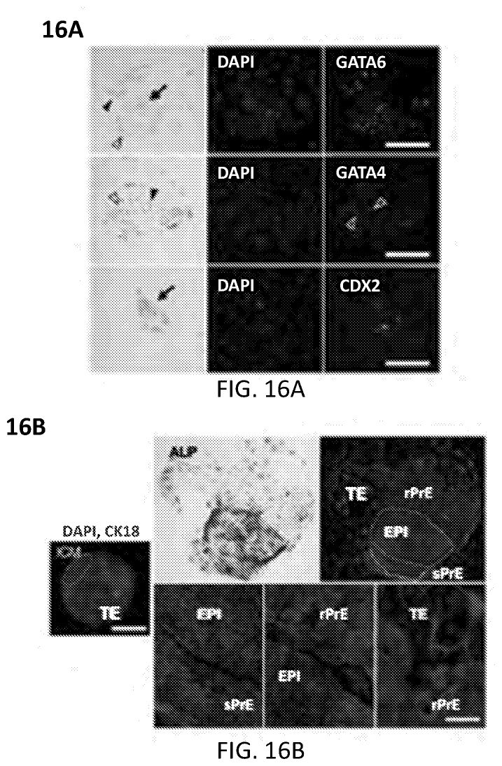

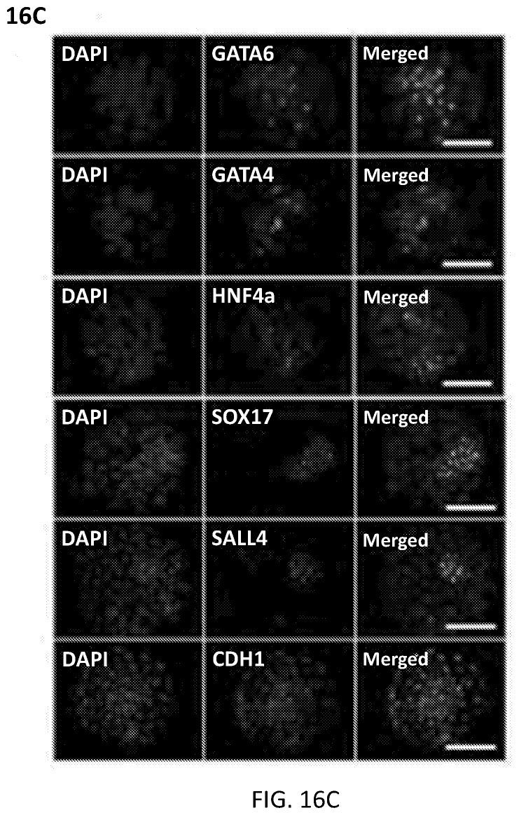

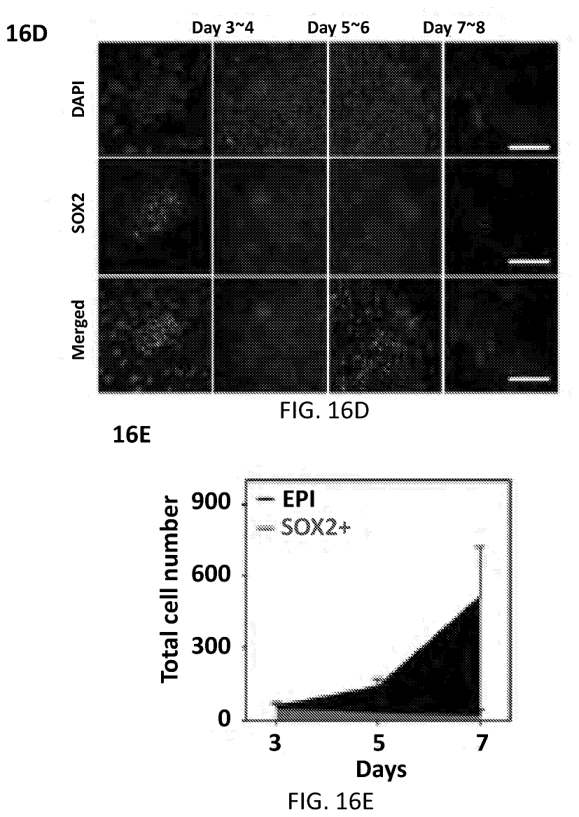

A central assumption behind the failure to establish pESC is a rapid loss of pluripotency in primary outgrowths. The whole blastocyst explants following attachment became flattened and spread out within 2 days of culture (FIG. 12a). As primary outgrowth expanded. TE cells began to first emerge and then underwent dramatic morphological changes, becoming larger and flatter, and soon-after undergoing apoptosis (FIG. 12a). After 5 days, a population of round and dispersed epithelial cells emerged as a discrete cell layer bordering the ICM (hereafter called “EPI”) cells (FIG. 12a). Majority of EPI cells were SOX2 positive (18/21) but only a few co-expressed NANOG (4/21) (FIG. 12b), similar to the staining pattern observed in the blastocyst (FIG. 12c). Notably, the large round cells, initially considered as TE cells stained positive for GATA6 (9/12) and CK18 but lacked CDX2 expression (FIG. 12b). The expression of GATA4, a later marker of the PrE− was also detected in few small round cells (4/7) (FIG. 16a), confirming two distinct PrE progenitors expressing GATA factors in primary outgrowths. These subpopulations, small and large PrE were distinguishable based on cell morphology and by their expression of CK18 (FIG. 16b). Although initial explants could be established from early blastocysts (day 5-6), late blastocysts (fully expanded or hatched, day 7-8), where the ICM and TE lineages were discernable (FIG. 12c;FIG. 16c) established stable PrE populations (FIG. 12d; 12e) and were used for further studies.

Initially, NANOG or GATA4 positive (+) cells were mostly undetectable, but cytoplasmic GATA4 expression appeared in the periphery of the early ICM outgrowths by d3 of culture (FIG. 12f). Intriguingly, NANOG/GATA4 co-positive cells that lined the side of EPI outgrowth gradually increased by 5 days, and by d 7>90% of GATA4+ cells co-expressed NANOG (FIG. 12f). In contrast, the expression of NANOG was detected in few, if not at all in EPI cells, while the SOX2 expression was progressively decreased with time, indicating the loss of pluripotency (FIG. 16d; FIG. 12e,g). Besides GATA factors, SALL421 a key stemness marker of XEN cells was expressed in the nuclei of the PrE cells that had a small and compacted appearance. A large fraction (˜75%) of SALL4+ cells had nuclear foci of intense histone 3 lysine 27 trimethylation (H3K27me3), a hallmark of the inactive X in female outgrowth22 (FIG. 12h). Consistent with this observation, XIST levels were 2-fold higher in SALL4+ PrE cells than EPI cells (FIG. 12i), which reflects the lineage specific dynamics of H3K27me3 accumulation on the X-chromosome, and could be the consequence of the co-expression of SALL421.

ii. Cellular Properties and Molecular Signature of Pig XEN Cells

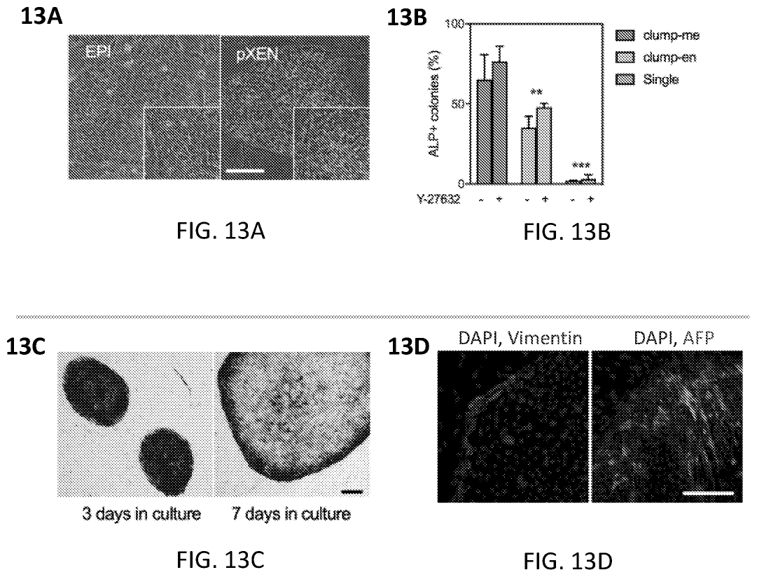

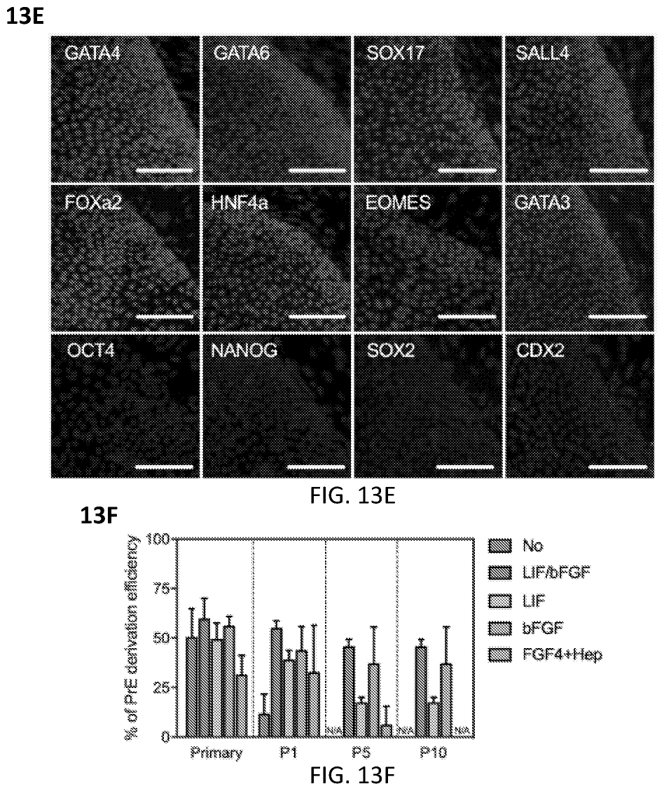

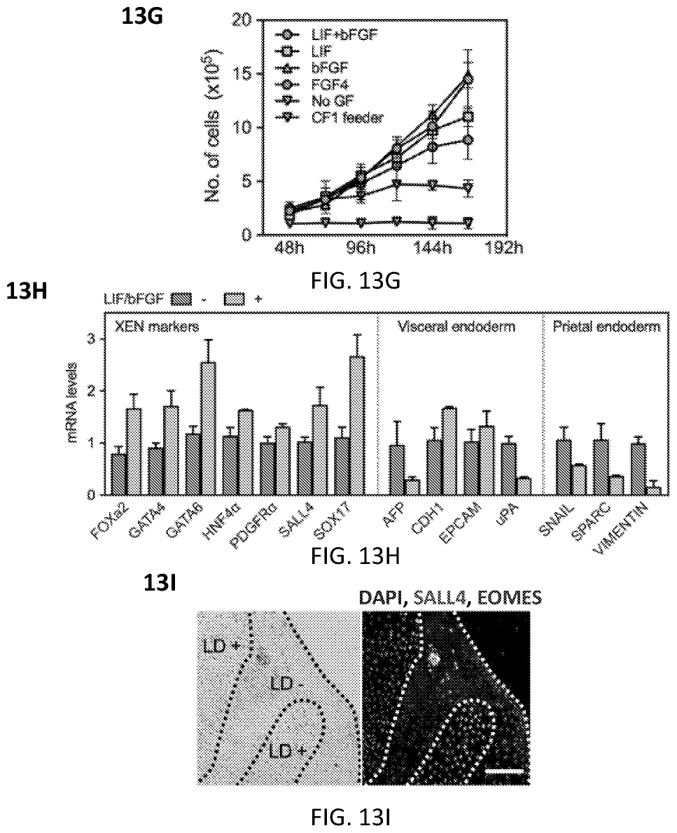

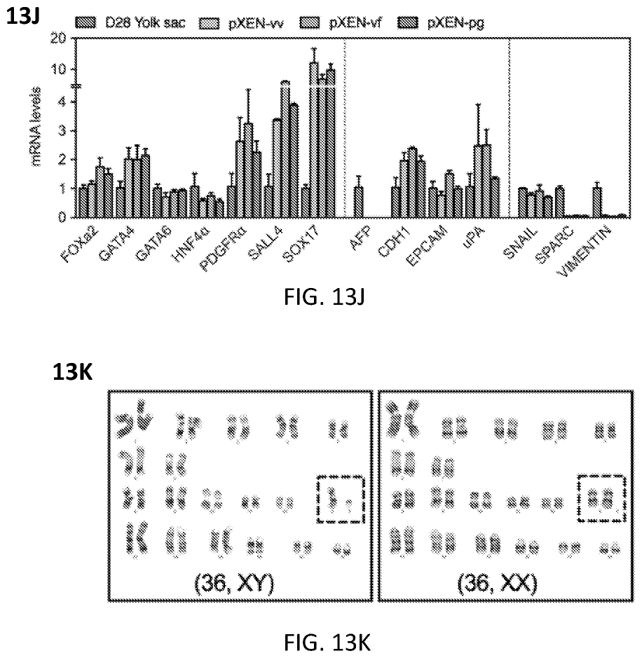

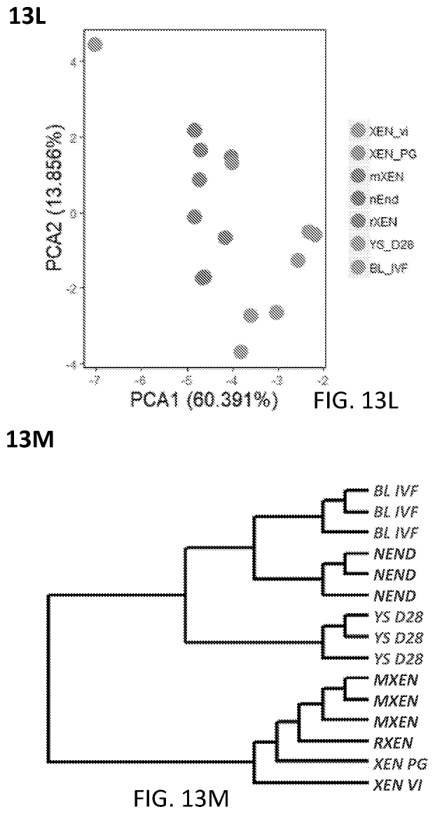

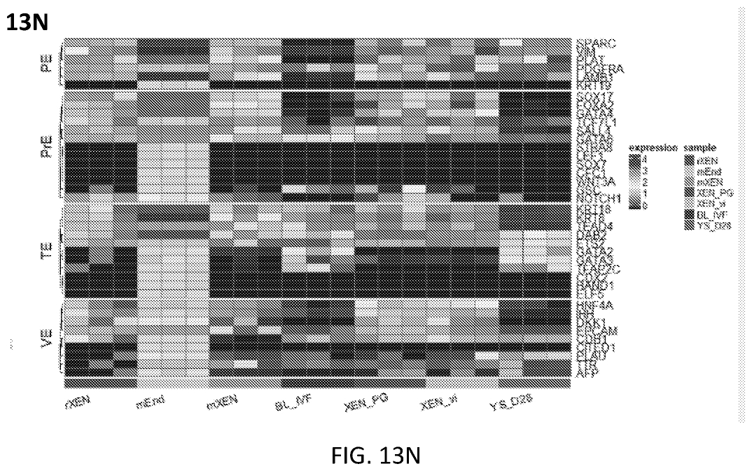

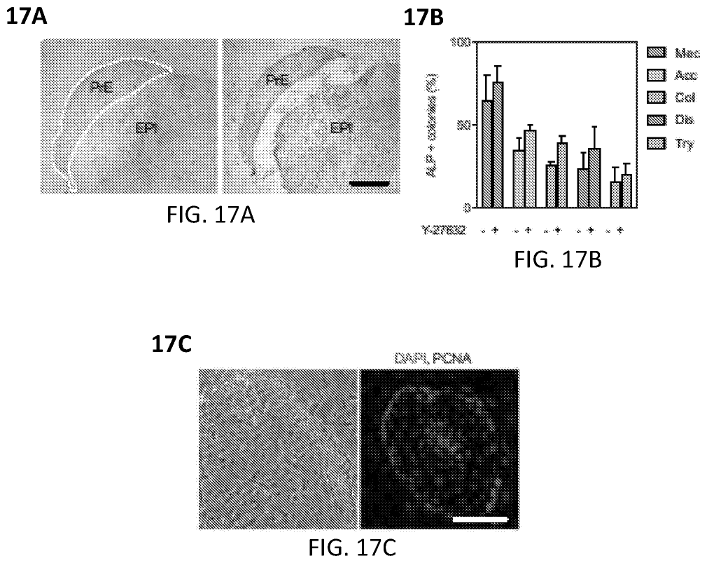

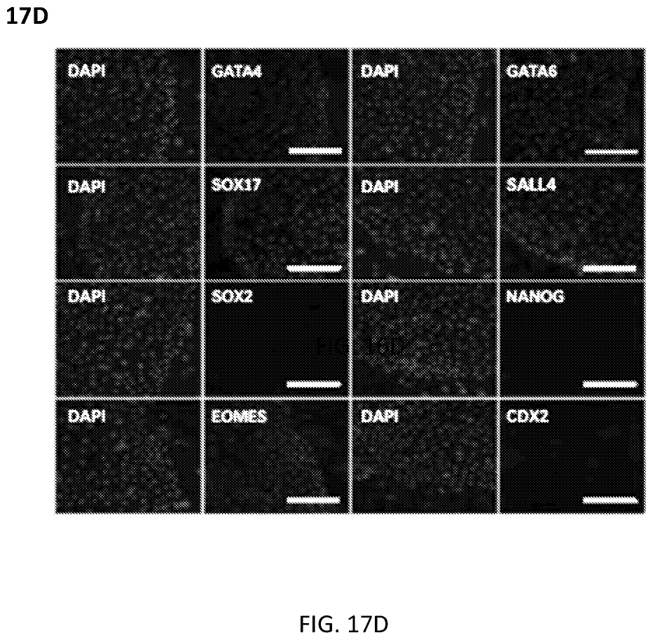

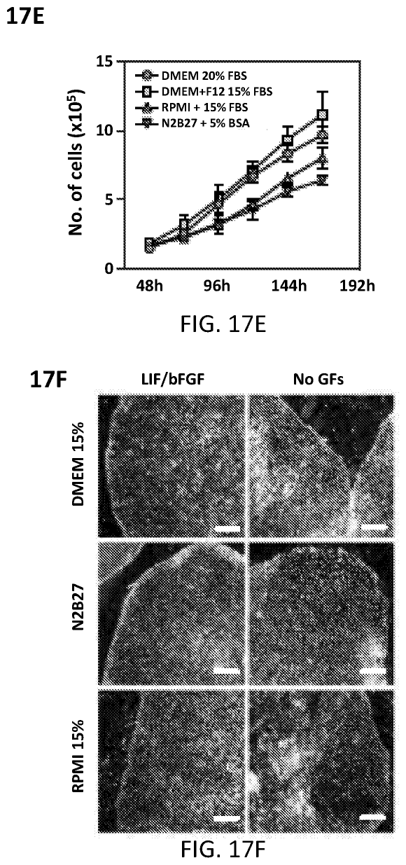

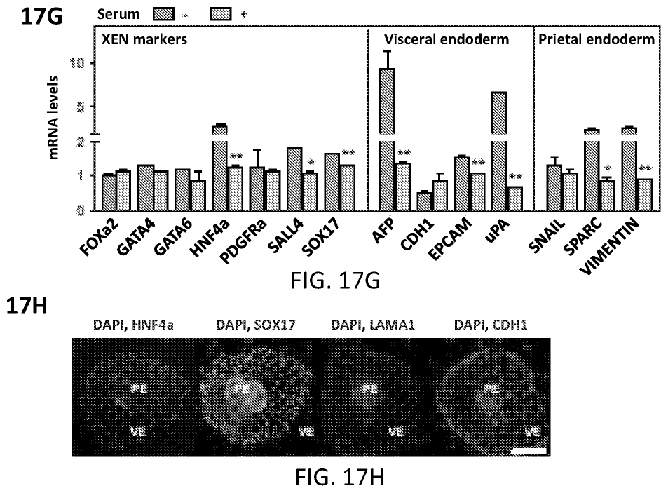

Self-renewal of XEN cells is dependent on Sall4 expression. The emergence of distinct SALL4+ PrE population in primary outgrowths have prompted us to attempt derivation of pXEN cells. After 7-9 days of culture. PrE cells began to emerge in primarv outgrowths and could be clearly demarcated based on their morphology and allowing for easy dissociation from the EPI cells (FIG. 17a). Both EPI and PrE colonies displayed a distinct morphology following serial passages (FIG. 13a). Consistent with previous findings, the EPI colonies underwent spontaneous differentiation toward a fibroblast- or neuron-like appearance by passages 5-7. The colonies from PrE-derivatives on the other hand, were more stable in culture. The colonies were propagated as flattened colonies and passaged as clumps by mechanical or enzymatic dissociation (FIG. 13b), but did not survive passage as single cells, even when treated with ROCK inhibitor Y-27632 (FIG. 13b; FIG. 17b). Following sub-passage, the PrE colonies initially appeared as a homogenous colony of cells and grew as a single sheet monolayer. Upon serial passaging two distinct populations emerged; a cobble-stone morphology in the center of colony, and an epithelial sheet-type cells at the borders of the colony (FIG. 17c). The cells at the periphery were strongly alkaline phosphatase (ALP) positive (FIG. 13c) and exhibited rapid proliferation as confirmed by PCNA staining (FIG. 17c). The density of the feeder cells influenced the colony stability with the optimal densities ranging from 3-4×10⁴ cells per cm². Lower feeder densities (<2×10⁴ cells/cm²) resulted in differentiation of cells with the expression of VIMENTIN (FIG. 13d), and at high density (>1×10⁵ cells/cm²), the cultures were more closely packed and showed reduced replating efficiency. The cells expressed PrE-specific markers (GATA4, GATA6, SOX17, SALL4, FOXA2, and HNF4A) with no expression of pluripotent markers (OCT4, SOX2, and NANOG) (FIG. 13e; FIG. 17e). Notably, NANOG was no longer detected upon passaging indicating a possible role for NANOG only in early PrE specification. While CDX2 is not detectable, other TE-markers EOMES and GATA3 were expressed, consistent with the role of the latter in endodermal specification. Taken together, the molecular signature confirmed the established colonies as XEN cells.

The growth factor requirements of pXEN cells were tested based on observations from mouse. Withdrawal of either LIF, bFGF or both, had no impact on primary PrE induction. However, in the omission of both, the cells failed to expand into stable cell lines confirming the growth factor responsiveness (FIG. 13f). The colonies that arose in the LIF or FGF4 alone did not proliferate as rapidly as cells cultured with either bFGF, or both LIF and bFGF (FIG. 13g). Omission of both growth factors resulted in a dramatic reduction in colony formation, with low expression of XEN marker genes FOXa2, GATA4, GATA6, HNF4a, PDGFRa, SALL4 and SOX17, and high expression of VE- (AFP and UPA), and PE-genes, (SNAIL, SPARC, and VIMENTIN), consistent with spontaneous differentiation (FIG. 13h). The XEN cells can be stably maintained in serum-free N2B27-based defined medium with lower degree of cellular differentiation and expression of VE- and PE-related genes, however requiring a longer cell doubling time (FIG. 17f; 13g). One interesting finding is the presence of characteristic lipid droplets in the cytoplasm of pXEN cells (FIG. 13a), which readily disappeared when plated in the absence of growth factors or feeder cells with a concomitant loss of SALL4 expression, but no change in EOMES expression (FIG. 13i). Although little is known about the mechanisms mediating the presence of lipid droplets, this feature could be leveraged as a non-invasive marker of SALL4+ cells.

Based on these preliminary trials, putative XEN cell lines were established from in vivo-developed (vi, n=4), in vitro-fertilized (vf, n=13), and parthenogenetically activated (pg, n=14) porcine blastocysts that exhibited stable morphology and marker expression, irrespective of the origin of embryos (FIG. 13j). The pXEN cells were maintained with proliferative potential in culture for extended passages (>50 passages), and were karyotypically normal (FIG. 13k). Transcriptomic analysis of pXEN cells expressed characteristic XEN cell repertoire and clustered closely with rodent XEN cells (FIG. 13l, 13m, 13n). Importantly, no teratoma development was observed in any recipient mice transplanted with the six robust pXEN cell lines ranging from 1×106 to 107 cells (Table 1) indicating that all injected pXEN cells were committed and not pluripotent cells.

TABLE 1
Teratoma assay for determining the potency of pig XEN cells. Six
to eight week-old (BRG, BALB/c-Rag2null IL2rgnuIl and NIH-III,
Cr:NIH-bg-nu-Xid) male mice were used to perform teratoma assay.
Before transplanting, cells were incubated for 2 hr in medium
supplemented with Y27632 (10 μM) and were suspended with Matrigel
matrix. Six XEN cell lines at passage 5-25 that were transplanted
subcutaneously into 6-8 weeks old immunodeficient mice (n =
26). Animals were monitored for 30 weeks or longer. However, teratoma
formation in all six lines tested was not detected.

| | | | No. | No. |
|---|---|---|---|---|
| | | | animals | teratoma |
| pXEN lines | Injected cell No | Stains | transplanted | developed |
| Xnt^{Col1A:GFP}#3-2 | 5 × 10⁶ | BRG | 2 | 0 |
| | | NIH-III | 2 | 0 |
| | 10 × 10⁶ | NIH-III | 1 | 0 |
| Xnt^{Col1A:GFP}#6-1 | 5 × 10⁶ | BRG | 1 | 0 |
| | | NIH-III | 1 | 0 |
| Xvv#2 | 1 × 10⁶ | BRG | 2 | 0 |
| | | NIH-III | 1 | 0 |
| | 10 × 10⁶ | BRG | 1 | 0 |
| Xvv#9 | 1 × 10⁶ | BRG | 2 | 0 |
| | 10 × 10⁶ | NIH-III | 2 | 0 |
| | | BRG | 2 | 0 |
| Xpg#1 | 1× 10⁶ | BRG | 2 | 0 |
| | | NIH-III | 2 | 0 |
| | 10 × 10⁶ | BRG | 2 | 0 |
| | | NIH-III | 2 | 0 |
| Xpg#4 | 5 × 10⁶ | BRG | 1 | 0 |
| | | | 26 | 0 |

iii. Contribution of pXEN Cells to Chimeras

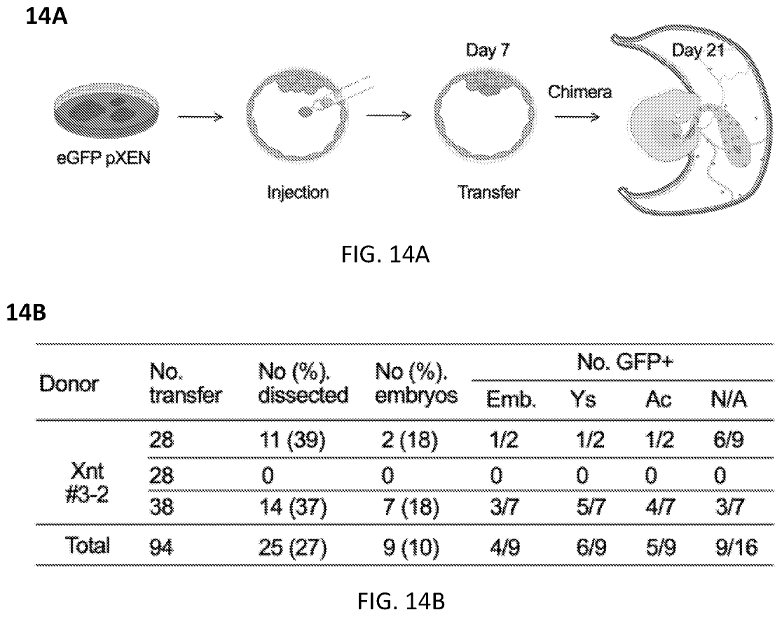

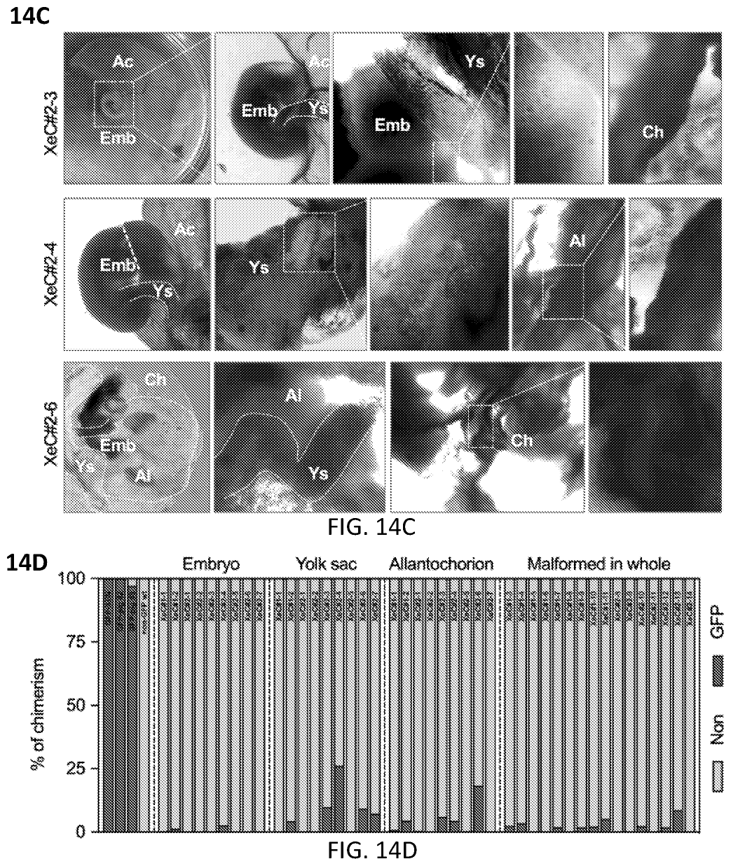

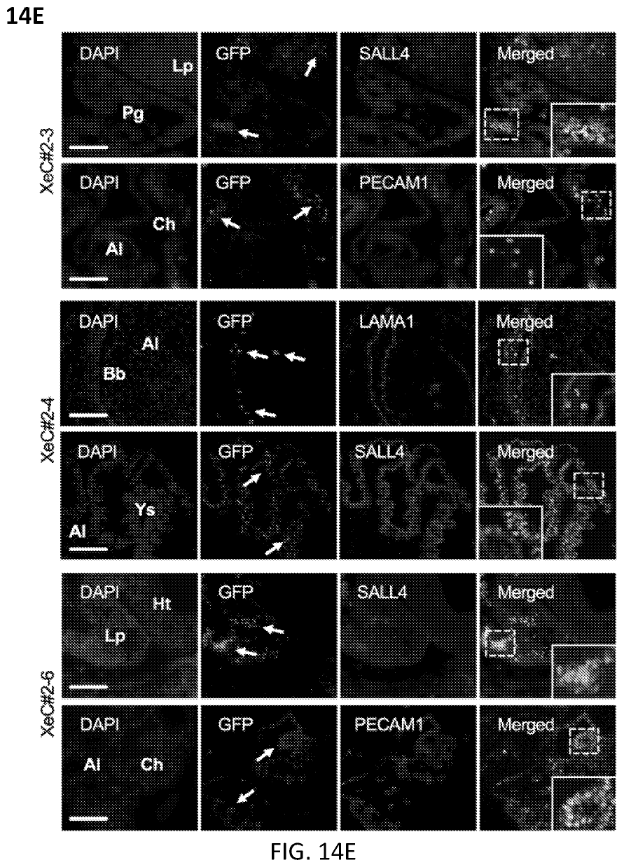

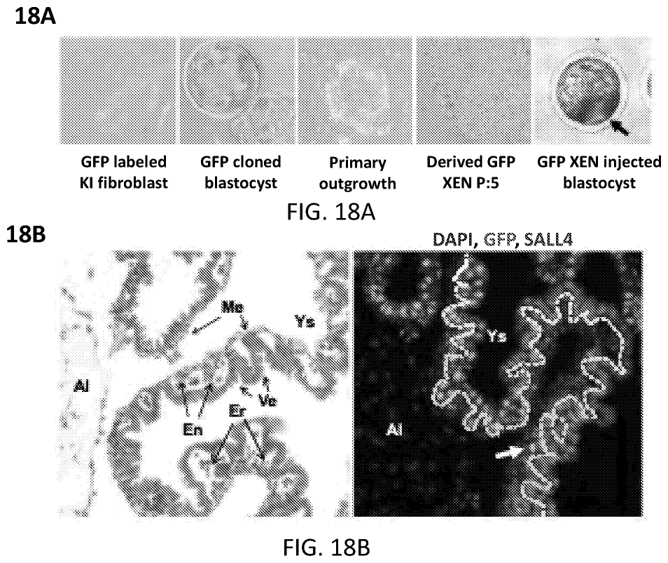

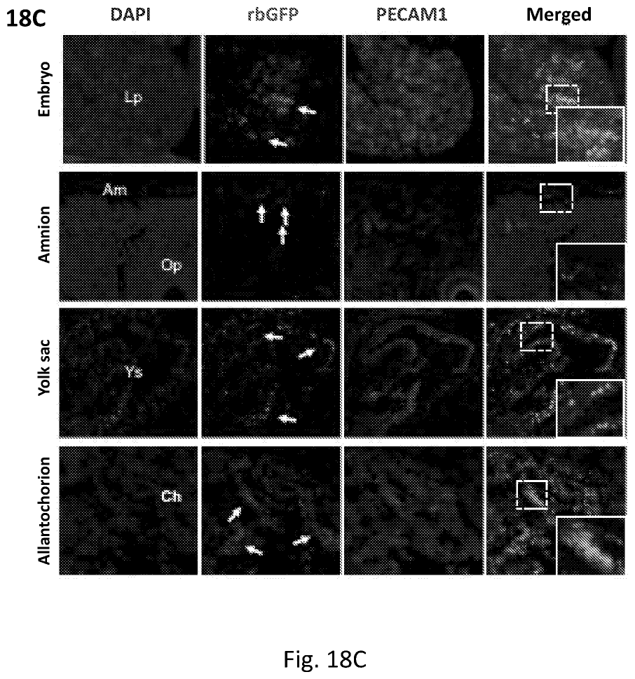

Mouse XEN cells contribute to PE, whereas rat XEN cells incorporate into both VE and PE lineages in chimeras. Given these disparities, we evaluated the properties of pXEN cells in chimera studies (FIG. 14a). To facilitate lineage tracing, a novel reporter pXEN cell line was generated by knocking-in a constitutive human UBC promoter driven GFP reporter downstream of the pCOL1A1 locus (hereafter, pCOL1A:GFP) using CRISPR/Cas system as previously described (FIG. 18a). Labeled pXEN (Xnt pCOL1A:GFP #3-2) cells were injected as single cells or 5-10 cell clumps into parthenogenetic embryos at the morula (Day 4) or early blastocyst stages (Day 5). Cells injected as clumps efficiently integrated into host embryos (77.3 to 85.7%) than individual cells (37.5 to 47.4%); and cells injected at the blastocvst stage showed better incorporation into ICM (85.7%) than injection at morula stage (77.3%) (Table 2). To evaluate in vivo chimeric development, pXEN cells were similarly injected as clumps into host blastocysts (n=109) and the resulting re-expanded blastocysts following overnight culture (n=94) were transferred into 3 recipient sows (FIG. 14b). A total of 25 fetuses (27%) were retrieved from 2 recipients on days 21 (FIG. 13b). Among the recovered fetuses, the injected GFP+ cells were found in the yolk sac (6/9) and the fetal membranes (5/9), and a small group of GFP+ cells were observed in one embryo (1/9) (FIG. 14b). Notably, GPF+ cells extensively contributed to yolk sac in two chimeras (XeC#2-3 and XeC#2-4) with a moderate signal in allantochorion (FIG. 14c). The GFP+ cells observed in embryos were from pXEN cells and not due to auto-fluorescence as confirmed by genomic PCR. Quantification of GFP+ cells by qPCR confirmed XEN cell chimerism at 1.7% in 2 embryos, and at 12.9% in the yolk sac, and 8% in the allantochorion, signifying active integration and proliferation of pXEN cells during embryogenesis (FIG. 14d). As shown in FIG. 14e, immunostaining with the anti-GFP antibody identified GFP+ cells in the embryonic gut of 3 chimeric fetuses (XeC#1-2, XeC #2-3, and XeC #2-6). The GFP+ donor cell population integrated predominantly into the visceral endodermal layers, but rarely into the outer mesothelial layers or endothelial cells in the yolk sac (FIG. 14e middle; FIG. 18c), and to a minor extent populated amnion, allantois, chorion (FIG. 14e), and gut endoderm (Table 3). Overall, the chimerism frequency of the pXEN cells was rather high (60%).

TABLE 2
Integration of pXEN cells to pig blastocysts.

| Single/ | | No. | No (%). | No. (%) | | | | |
|---|---|---|---|---|---|---|---|---|
| clumps | Stages | Injected | Blastocyst* | contributed | | into ICM | | into TE |
| Single | Morula | 26 | 24 (92.3) | 9 | (37.5) | 5 | (20.8) | 4 (16.7) |
| | Blastocyst | 22 | 19 (86.4) | 9 | (47.4) | 5 | (20.8) | 4 (21.1) |
| Clump | Morula | 24 | 22 (91.7) | 17 | (77.3) | 8 | (36.4) | 8 (36.4) |
| | Blastocyst | 27 | 21 (77.8) | 18 | (85.7) | 14 | (66.7) | 4 (19.0) |

*The number in blastocyst injection is the number of re-expanded blastocysts on 2 day following injection. pXEN cells (Xnt Col1A:GFP#3-2) were injected as individual cells or as small clumps after Accutase-treatment.

TABLE 3
In vitro development of pig cloned embryos. All the cells
as nuclear donors were from the same origin (FFwt #6: a
female Ossabow fetal fibroblast), except for Xvv#9
that was derived from an in vivo embryo (crossbred). There
was no statistically significance between the groups.

| | | No. | No (%). | No (%). | |
|---|---|---|---|---|---|
| Donor cells | Cell type | reconstructed | 2-4 cells | blastocysts | |
| FF^{wt}#6 | Fibroblast | 25 | 22 (88.0) | 8 | (32.0) |
| FF^{Col1A:Attp}#6-1 | Fibroblast | 107 | 87 (81.3) | 37 | (34.6) |
| FF^{Col1A:GFP}#3 | Fibroblast | 40 | 34 (85.0) | 15 | (37.5) |
| Xnt^{Col1A:GFP}#3-2 | XEN | 95 | 79 (83.2) | 36 | (37.9) |
| Xvv#9 | XEN | 32 | 23 (71.9) | 14 | (43.8) |

iv. Generation of Viable Cloned Offspring from pXEN Cells Via SCNT

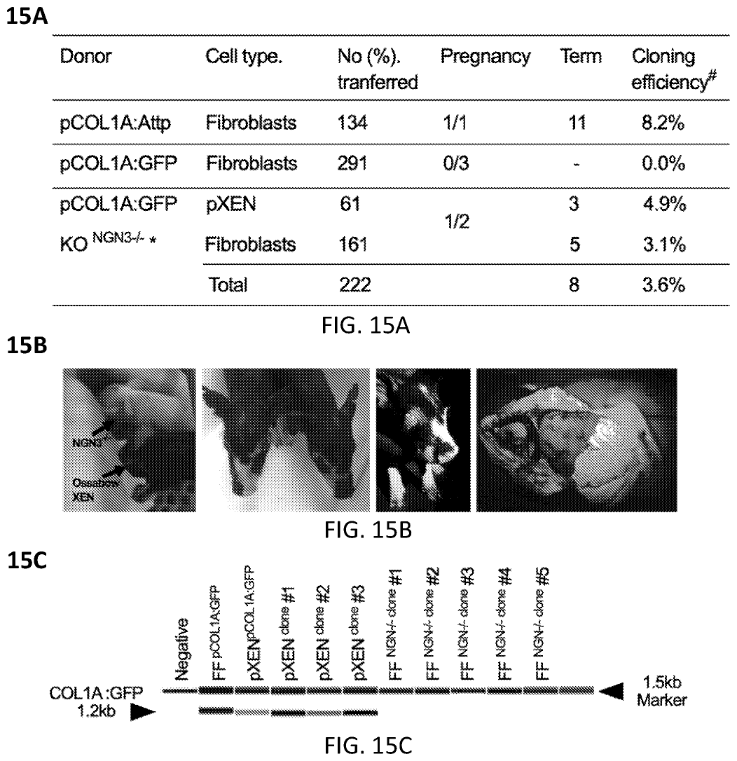

In an effort to test the utility of pXEN cells as nuclear donors, SCNT was performed with the pXEN cells used in the chimera assay (above), alongside previously published crossbred knock-out fetal fibroblasts (FF NGN3−/−) as controls. A total of 222 cloned embryos reconstituted from pXEN (Xnt pCOL1A:GFP #3-2, n=61) and FF (n=161) were co-transferred into two surrogate gilts to exclude confounding variables associated with recipient animals affecting the outcome. Following embryo transfers, one pregnancy was established, and delivered 8 cloned piglets at term. Three of the 8 piglets were GFP positive and black coated (4.9%) confirming the COL1A:GFP Ossabaw XEN cell origin, while 5 piglets were white coated and GFP negative from the control fibroblasts (3.1%) (FIG. 15A). As expected, the piglets exhibited ubiquitous expression of GFP in all tissues (FIG. 15B). The genotype of the offspring was confirmed by PCR (FIG. 15C). In addition to this, multiple rounds of SCNT was performed with FF pCOL1A:GFP (#3) from which the XEN cells were derived. Despite being genetically identical, no offspring were obtained from founder GFP fibroblasts, but the derived XEN cells served as efficient donors in SCNT.

3. Discussion

Establishment of pESC from embryonic explants has largely been unsuccessful despite nearly three decades of investigation. As shown by multiple groups, the EPI fraction of the primary explants fail to proliferate, and the cultures are rapidly overtaken by proliferating ExE cells. That said, there were no published reports that temporally followed the fate of the ExE derived lines in culture, nor have they been adequately characterized; although, the equivalent lines from mouse have been thoroughly characterized. This report for the first time takes a systematic and in-depth look at the derivation, establishment, and characterization of XEN cells from PrE.

During early mouse embryo development. NANOG is expressed in EPI cells and excluded from GATA4+ PrE cells in embryo. This seems counterintuitive given the mutual antagonism between NANOG and GATA4 that facilitate key cell-fate decisions between EPI and PrE, respectively. Indeed, several lines of evidence support the expression of NANOG in pig hypoblast, which is contrary to the mouse model. Emergence of PrE population with co-expression of GATA4/NANOG appears to represent an early step in PrE specification, highlighting mechanistic differences in early lineage specification between mouse and pig. That said, the establishment of pXEN cells, culture characteristics, and the resulting molecular signatures (including high expression of FOXa2. GATA4, GATA6, HNF4a, PDGFRa, SALL4 and SOX17) are shared with rodent models, with the exception of failure to establish XEN cells in FGF4-based medium, and intolerance to dispersal as single cells.

Generation of embryonic chimeras has been considered as the most stringent test of stem cell differentiation potential in vivo. This study demonstrates that despite the lack of pESC, it is possible to generate embryo-derived stem cell lines with PrE-like properties as confirmed by lineage-restricted plasticity in the resulting chimeras, which were not irrevocably fixed (e.g., yolk sac, placenta, gut endoderm). This indicates that the pXEN cells are in a less committed endodermal naïve state. In the pig, freshly isolated ICMs are capable of widespread tissue contribution, including germline colonization in chimeras. Despite this, the pluripotent EPI or iPS cells were preferentially engrafted into extraembryonic tissues. It is likely that in the absence of defined conditions, embryonic outgrowths are unstable and transition to a XEN-like state. Future chimera trials will be performed in embryos that lack key gate-keeper genes (for e.g., SALL4), where the relative contribution of pXEN cells to embryonic and ExE endodermal lineages are expected to be higher when compared to current experiments performed with wild type embryos.

In vivo generation of human organs via interspecies chimeras between human and pigs via blastocyst complementation has been acknowledged as a source of donor organs for life-saving regenerative medicine applications. Evidence gathered in the present study demonstrates the engraftment potential of pXEN cells with lineage restricted cell fate. When such experiments are performed with human XEN cells, the potential contribution to endodermal organs will provide an on-demand source of human endodermal cells in pig hosts. These findings make the use of pXEN cells a particularly attractive choice to generate tissue-specific chimeras for endodermal organs, while limiting unwanted contribution to undesirable organs (e.g., germ cell or neural lineage) in interspecies chimeras—a likely outcome with the use of ESC/iPS cells.

Another advantage of the pXEN cells is the competency to generate live animals via SCNT. This is especially attractive in complex genome editing and genetic engineering applications where long-life span in culture is desirable. As evidenced from this study, genetically modified fibroblast cells failed to generate live offspring, whereas, the pXEN cells derived following cloning of the FFs were able to generate live offspring at a relatively high efficiency (4.9%). One potential explanation is the epigenetic disruption caused by transfection that may have compromised embryonic development. The pXEN cell derivation processes, has potentially reset the genome to a state that allows full-term development. It remains to be seen, if this could be applicable to other cells which failed to generate live offspring. Taken together, we argue that the derivation of pXEN cells fulfils a longstanding need in the livestock genetics for a stem cell line of embryonic origin that can be reliably and reproducibly generated, are stable in culture, have the potential to contribute to chimeras, and are a good source for creating cloned animals.

4. Materials and Methods

i. Establishment and Maintenance of Pig XEN Cells

Embryonic explants and XEN cells were cultured on a feeder layer of early passage (n=3) CF-1 mouse embryonic fibroblasts (MEF) cells mitotically inactivated by treatment with mitomycin-C (3 hr, 10 μg/mL). A day before seeding the embryos or XEN cells, the feeders were plated in MEF medium based on high-glucose Dulbecco's modified Eagle medium (DMEM; Gibco, Grand Island, N.Y.) supplemented with 10% (v/v) fetal bovine serum (FBS; HyClone Laboratories Inc., Logan Utah, USA) on 0.1% (v/v) gelatin-coated four-well plates (Nunclon, Roskilde. Denmark) at a density of 3-5×105 cells per cm2. At least 2 hr before the start of the experiment, the MEF medium was aspirated and replaced with ‘standard ES medium’ which included DMEM/Nutrient Mixture Ham's F12 (DMEMF-12; Gibco) supplemented with 15% ES-qualified fetal calf serum (FCS; HyClone Laboratories Inc.), 1 mM sodium pyruvate, 2 mM L-glutamine, 100 units/mL penicillin-streptomycin, 0.1 mM 2-β-mercaptoethanol, 1% non-essential amino acids (NEAA; all from Gibco), with various combination of growth factors, 10 ng/mL human recombinant leukemia inhibitory factor (hrLIF; Milipore, Bedford, Mass.) and 10 ng/mL human recombinant basic fibroblast growth factor (hrbFGF; R&D Systems, Minneapolis. Minn.). Other media combinations that were tested include RPMI 1640 or N2B27 serum free medium (1:1 ratio of DMEM/F12 and Neurobasal medium plus N2 and B27, all from Gibco), with a combination of 5 ng/mL LIF and/or 10 ng/mL bFGF, or 25 ng/mL human recombinant fibroblast growth factor 4 (hrFGF4; R&D Systems) and 1 μg/mL heparin1. Following initial plating, attachment and outgrowth development, the medium was refreshed on d3, followed by media exchange every 2 days. After 7-8 days of culture, the primary outgrowths were mechanically dissociated into small clumps, and transferred onto fresh feeders for passaging. The pXEN cells were cultured at 38.5° C. in 5% 02 and 5% CO2, with the culture medium being refreshed every other day and passaged at L20 every 7-8 days. Cells were passaged as clumps by gentle pipetting following 10 min digestion with Accutase (Gibco). Before routine passaging and freezing, cells were cultured with Rho Kinase (ROCK) inhibitor Y-27632 (10 μM: StemCell Technologies,

Vancouver, Canada) at least 2 hr prior to dissociation2. Each XEN cell line was frozen in FBS based medium supplemented with 8% (v/v) DMSO and recovered with high viability. In order to determine chromosomal stability in long term culture, cytogenetic analysis was performed by Cell Line Genetics

ii. Alkaline Phosphatase Staining

The cells were fixed with 4% (w/v) paraformaldehyde for 3 mm at room temperature (RT) and were washed three times with DPBS. Alkaline phosphatase (ALP) staining was performed with a BCIP/NBT Alkaline Phosphatase Colour Development Kit following the manufacturer's instructions. The cells were examined using an inverted microscope.

iii. In Vitro Differentiation of XEN Cells into Parietal or Visceral Endoderm:

The pXEN cells were differentiated by means of embryoid body (EB) formation and treatment with small molecules and factors. pXEN cells were dissociated as clumps, washed, and resuspended in medium (DMEM/F12 plus 15% FBS) as hanging drops on the lid of a 60 mm dish, and cultured for 5 days, during which time spheroids were formed. To direct pXEN cells differentiation into either visceral endoderm (VE) or parietal endoderm (PE), accutase-dissociated single cells (2×105 cells per cm2) were seeded onto a laminin- or fibronectin-coated 6 well plate in N2B27 medium supplemented with the respective differentiation factors and/or chemicals. For example, for differentiation into VE, the cells were treated with CHIR99021 (10 μM, STEMCELL Technologies Inc.) and BMP4 (50 ng/mL, R&D) for activating Wnt/β-catenin pathway; for differentiation into PE, Folskolin (50 μM) and dbcAMP (1 mM) for activating the cyclic adenosine monophosphate (cAMP) signaling pathway were utilized. Differentiation medium was replaced every two days, and cells were processed for analysis on day 12.

iv. Methods for Embryo Production and Manipulation

The in vivo and in vitro embryo production were performed as described previously. For generating parthenote, in vitro fertilized embryos, and for performing somatic cell nuclear transfer (SCNT), cumulus-oocyte complexes were purchased from a commercial supplier (De Soto Biosciences, Seymour, Tenn., USA). After in vitro maturation, the cumulus cells were removed from the oocytes by gentle pipetting in a 0.1% (w/v) hyaluronidase solution. Briefly, for In vitro fertilization (IVF), pre-diluted fresh semen (Duroc; Progenes) was centrifuged twice at 20 g for 3 min in DPBS containing 0.2% BSA. The sperm pellet was adjusted to a concentration of 2×105 sperm per mL and co-incubated with matured oocytes in modified Tris-buffered medium containing 0.4% BSA for 5 hr in a humidified atmosphere (5% CO2 in air). Following three washes, putative zygotes were cultured and maintained in PZM3 medium in a low oxygen air (5% O2 and 5% CO2 in air). For obtaining in vivo embryos, donor animals were synchronized using Regumate and artificially inseminated at 12 and 24 hr following the observation of first standing estrus. On days 5-7 post-insemination, in vivo embryos were recovered by flushing oviduct with 35 ml of TL-Hepes buffer containing 2% BSA under general anesthesia. For SCNT, fetal fibroblasts (FF) were synchronized to the G/GO-phase by serum deprivation (DMEM with 0.2% FCS) for 96 hr, and pXEN cells were mitotically arrested by serum free medium (N2B27 with 1% BSA) for 48 hr followed by incubation with aphidicolin (0.1 μM) for 12 hr. Enucleation was performed by aspirating the polar body and the Mil metaphase plates using a micropipette (Humagen, Charlottesville, Va., USA) in 0.1% DPBS supplemented with 5 μg/mL of cytochalasin B. After enucleation, donor cells were placed into the perivitelline space of an enucleated oocyte. Fusion of cell-oocyte couplets was induced by applying two direct current (DC) pulses (1-sec interval) of 2.1 kV/cm for 30 μs using a ECM 2001 Electroporation System (BTX, Holliston, Mass.). After fusion, the reconstituted oocytes were activated by a DC pulse of 1.2 kV/cm for 60 μs, followed by post-activation in 2 mM 6-dimethylaminopurine for 3 hr. After overnight culture in PZM3 with a histone deacetylase inhibitor Scriptaid (0.5 μM), the cloned embryos were surgically transferred into the oviduct. Parthenogenetic embryos were produced by the activation procedures used for SCNT.

v. Embryo Transfer

The surrogate recipients were synchronized by oral administration of progesterone analog Regumate for 14-16 days. Animals in natural estrus on the day of surgery were used as recipients for SCNT embryo transfers (into oviduct), and at days 5-6 after natural heat were used for blastocyst transfer (into uterus) for generating chimeras. Surgical procedure was performed under a 5% isofluorane general anesthesia following induction with TKX (Telazol 100 mg/kg, ketamine 50 mg/kg, and xylazine 50 mg/kg body weight) administered intramuscularly. Pregnancies were confirmed by ultrasound on day 27 following transfer. Cloned piglets were delivered at day 117 of pregnancy by natural parturition.

vi. RNA and DNA Preparations

For isolation of genomic DNA (gDNA) from cells and tissues, the QIAamp mini DNA Kit (Qiagen, Valencia, Calif., USA) was used according to the manufacturers' instructions. Total RNA was isolated using Trizol plus RNeasy mini kit (Qiagen) and mRNA from individual blastocysts was extracted using the Dynabeads mRNA Direct Kit (Dynal Asa, Oslo, Norway). Synthesis of cDNA was performed using a High Capacity cDNA Reverse transcription kit (Applied Biosystems: ABI, Foster City, Calif.) according to the manufacturers' instructions. The QIAseq FX Single Cell RNA Library kit (Qiagen) was used for Illumina library preparation and transcriptomics analysis.

vii. qPCR

Relative quantification of mRNA levels was carried out using SYBR Green technology on an ABI 7500 Fast Real-Time PCR system (Applied Biosystems). The thermal-cycling conditions are: 20 s at 95° C. followed by 40 cycles of 3 s at 95° C. and 30 s at 60° C. The primers were designed to yield a single product without primer dimerization. The amplification curves for the selected genes were parallel. All reactions were performed from three independent biological and two technical replicates. Two reference genes. ACTB and YWHAG were used to normalize all samples and the relative expression ratios were calculated via the 2-ΔΔ Ct method6. The primers used in qPCR are listed in Table 4.

TABLE 4
Primers and Antibodies
qPCR for mRNA expression analysis

| Gene | Forward | Reverse |
|---|---|---|
| ACTB | gtggacatcaggaaggacctcta | atgatcttgatcttcatggtgct |
| AFP | Cacctttccaggttccagaa | aaggggtgccttcttgctat |
| CK8 | Tctgggatgcagaacatgag | ggctgtagttgaagcctgga |
| CK18 | Gcaagttctgtggacaatgc | gccagctccgtctcatactt |
| CK19 | ctgaaggaagagctggccta | tcaacctccacactgacctg |
| CXCR4 | Cagcaagggtgtgagtttga | tccaaggaaagcgtagagga |
| CDH1 | Cacctcacgggaattgtctt | ttatcagcacccacgcaata |
| DKK1 | Aggctcttggaaccctgact | ccaaaggactcaaggcagag |
| EPCAM | ccaaaaggatggacctgaga | agcctgtagaccctgcattg |
| FOXA2 | ataaggagggcaagggaaaa | agtcaaaattcgcaggtgct |
| GATA4 | tctcggaaggcagagagtgt | caggcgttgcacaggtagt |
| GATA6 | Atcaccatcaccacccaagt | cgcgactctgtagactgtgc |
| GPC1 | Ccaggatgccagtgatgac | tggagcttttcttgctgacc |
| GSC | Gaagccctggagaacctctt | cggtttttgaaccagacctc |
| HNF4α | Ctcagcaacggacagatgtg | caggagcttgtagggctcag |
| NANOG | Cccccttcttcaactcaaca | cttcaggcccataaacctca |
| OCT4 | gctggagccgaaccccgagg | caccttcccaaagagaacccccaaa |
| PDGFRα | caggttggagggagatggac | agttgcggaggttggatt |
| RN18S | acaaatcgctccaccaactaaga | cggacacggacaggattgac |
| SALL4 | caggagtaccagagccgaag | acctcgggagacttggactt |
| SNAIL | Ttttcagcagccctatgacc | ccaggagagagtcccagatg |
| SOX2 | aacagcccagaccgagttaa | gttgtgcatcttggggact |
| SOX7 | Ggctagtgaaagccaactcg | tttgcctgccttgagagaat |
| SOX17 | Tggttgaatcttgaggtctgc | cagggtgtaggtgtgtgatga |
| SPARC | Ggaccatcagtcctctggaa | agttctgcgtctcccaaaga |
| TGFb1 | Gtcttcttcggacgttaccg | gcatgaggaggaggaacaaa |
| uPA | Aagggctctgacattccatg | ccggctcttacactgacaca |
| VIMENTIN | gtaccggagacaggtgcagt | ttccacggcaaagttctctt |

qPCR for Chimerism analysis

| Gene | Forward | Reverse |
|---|---|---|
| GFP | Aagttcatctgcaccaccg | tccttgaagaagatggtgcg |
| YWHAZ | agtaggttgggctccttgacac | gccgactgtgactttaaggtgc |

PCR for genotyping

| Gene | Forward | Reverse |
|---|---|---|
| pCOL1A1 | gcatggagagaaggcatgat | |
| Ubc promoter | | tcacagcgatccagaaagaa |
| NGN3 | caccagaccgagcagtcttt | ttggtgagtttcgcatcgt |

Antibodies

| Antigen | Antibody Source |
|---|---|
| AFP | Abcam (ab74663) |
| CDX2 | Abcam (ab76541) or Santa Cruz (sc19478) |
| EOMES | Santa Cruz (sc98555) |
| HNF4α | R&D Systems (AB41898) |
| SOX2 | Abcam (ab79351) or Santa Cruz (sc17320) |
| GATA4 | Santa Cruz (sc1237) |
| GATA6 | Santa Cruz (sc9055) |
| SALL4 | Santa Cruz (sc46045) |
| CDH1 | Antibodies-online (ABIN3209718) |
| GFP | Santa Cruz Biotech (sc9996) |
| LAMININ | Sigma Aldrich (L9393) |
| SOX17 | R&D Systems (AF1924) |
| SOX7 | R&D Systems (AF2766) |
| NANOG | Santa Cruz (sc33760) or Peprotech (500-P236) |
| OCT-4 | Santa Cruz (sc5279) |
| PCNA | Santa Cruz (sc56) |
| H3K27me3 | Epigentek (A4039-025) |
| VIMENTIN | Santa Cruz (sc6260) |
| Cytokeratin 8/18/19 | Abcam (ab41825) |
| Donkey anti-mouse | Santa Cruz (sc-2099 g) or Abcam (ab96878) |
| Donkey anti-rabbit | Santa Cruz (sc-2090) or Abcam (ab96894) |
| Donkey anti-goat | Santa Cruz (sc-2783) or Abcam (ab96935) |

viii. Data Access

A total of 12 RNA-seq data sets generated in this study have been deposited in the CNSA (https://db.cngb.org/cnsa/) of CNGBdb with accession code CNP0000388, and also NCBI Gene Expression Omnibus (GEO; http://www.ncbi.nlm.nih.gov/geo) under accession number GSE128149.

ix. Transcriptomics Analysis

RNA-seq reads were mapped to the pig reference genome (Sscrofal1.1) using HISAT27 (version 2.0.4) with parameters “hisat2-sensitive --no-discordant --no-mixed -I1-X 1000” and to the reference cDNA sequence using Bowtie28 with parameters “bowtie2-q --sensitive -dpad 0 --gbar 99999999 --mp 1.1 --np 1 --score-mn L,0,−0.1 −1 1 −X 1000 --no-mixed --no-discordant -p 1 -k 200”. Then the expression levels of each gene were calculated by the fragments per kilobase of exons per million fragments mapped (FPKM) using RSEM9 with parameters “rsem-calculate-expression --paired-end -p 8” based on the result of Bowtie2. The data of mouse and rat XEN cells were downloaded from GSE10615810 (mouse: GSM2830587, GSM2830588 and GSM2830589; rat: GSM2830591, GSM2830592 and GSM2830593) and the gene expression levels were calculated in the same way (the mouse and rat reference genome used were GRCm38.p6 and Rnor_6.0, respectively). The expression levels of mouse nEnd were downloaded from GSE1074211 (GSM271163, GSM271164 and GSM271165). Then the expression levels of all samples were combined to obtain the expression matrix. Final expression matrix was calculated by cross-species gene expression analysis as reported previously12. The expression values from mouse, rat and pig were transformed separately into relative abundance values: for each gene, the relative abundance value is the expression value divided by the mean of expression values within the same gene across samples in the same species. The final expression matrix was subjected to hierarchical clustering using R software. Development stage (PE. PrE, TE, VE and EPI)-specific genes were selected to do the subsequent analyses. They were mapped to the final expression matrix to do the PCA and heatmap analysis with R software.

x. Generating of a GFP-KI Reporter.

In order to establish green fluorescent protein (GFP) gene-based reporter XEN cell line, we used a site-specific knock in (KI) Ossabaw fetal fibroblasts. In order to facilitate KI at high frequencies, we have used a combination of small molecule inhibitor of NHEJ pathway (SCR7)13 and a pre-complexed Cas9 protein and sgRNA RNP complex to KI a ubiquitous promoter (UBC) driven GFP (Sanger Institute) downstream of a ubiquitously expressed COL1A1 locus to ensure stable expression of transgenes. After a day of transfection, the GFP-positive (GFP+) cells were sorted by flow cytometry (Becton Dickinson. Franklin Lakes. N.J., USA) and GFP+ single cells were replated into wells of a 96-well plate for expansion. After 10-15 days, individual colonies were washed, suspended in 20 μL of lysis buffer (50 mM KCl, 1.5 mM MgCl2, 10 mM Tris pH 8.0, 0.5% NP-40, 0.5% Tween-20 and 100 μg/mL proteinase K) and incubated for 1 h at 65° C. followed by heating the mixture at 95° C. for 10 min to inactivate the enzymes. The cell lysates (2 μL) were directly used as a template for PCR with screening primers (FIG. 16). Using this approach, we have identified>60% of the clonal lines showing stable integration of the transgene. The targeted-clones (hereafter called pCOL1A:GFP) with a strong and consistent fluorescence intensity as determined by fluorescence microscopy were frozen in 92% FCS and 8% DMSO, prior to use as nuclear donor cells. Using GFP labeled XEN cells, live animals were generated by SCNT.

xi. Chimera Assay

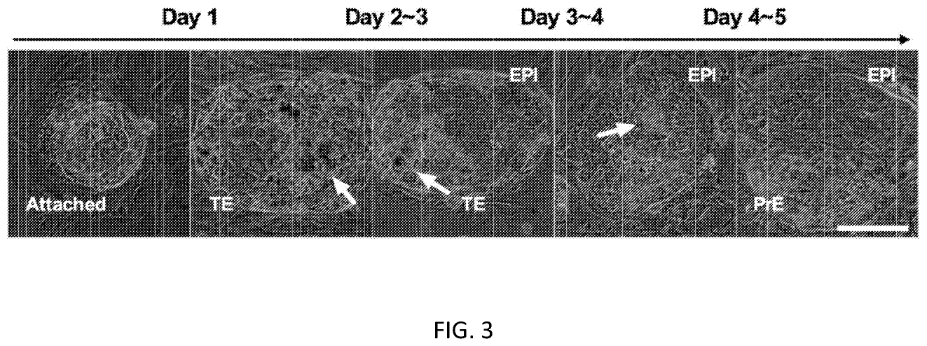

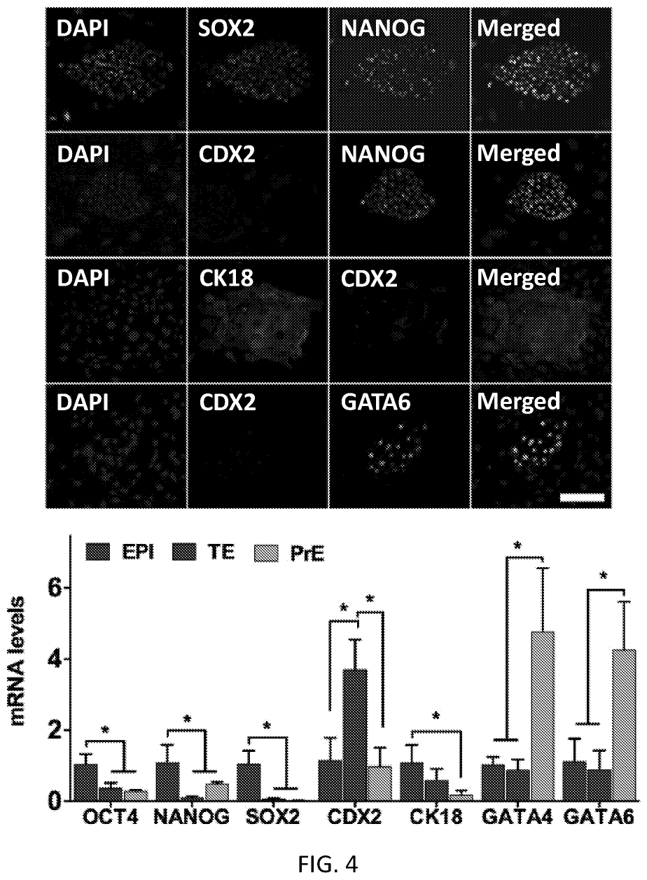

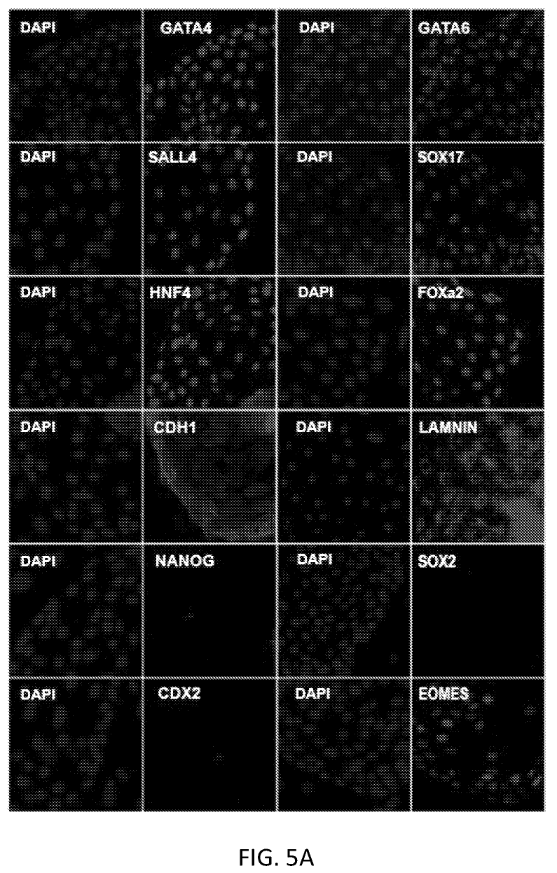

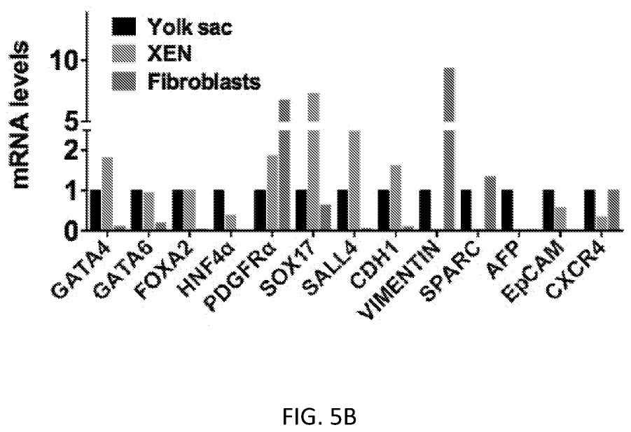

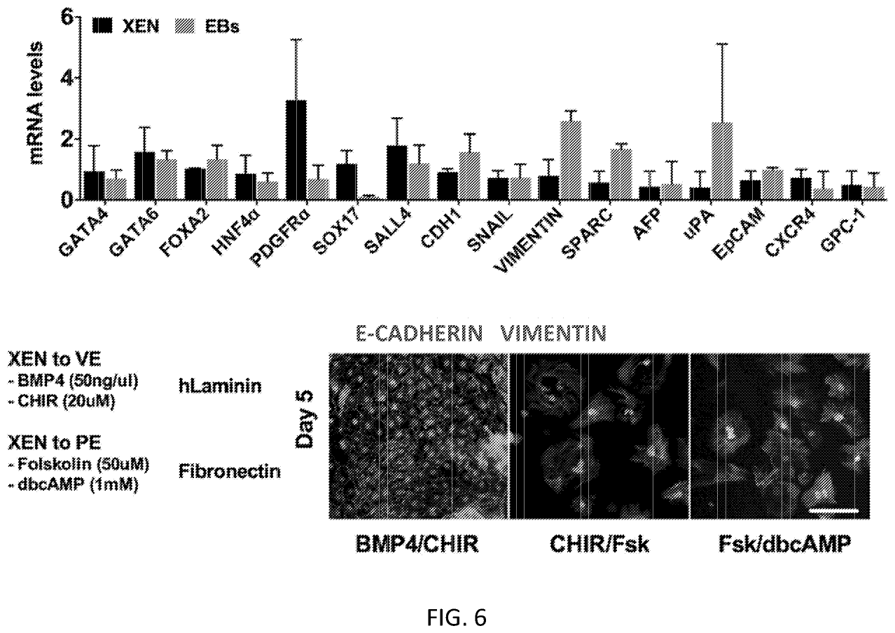

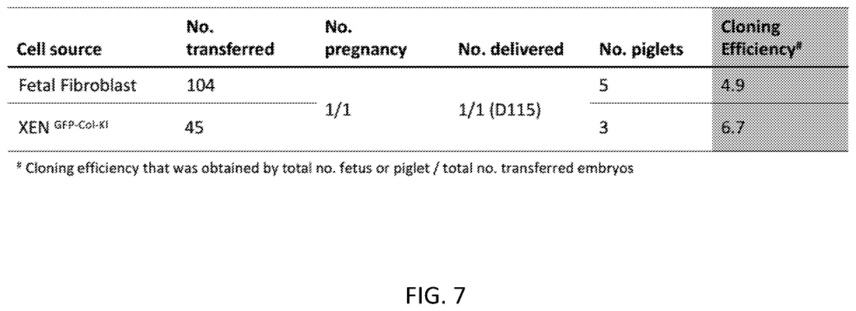

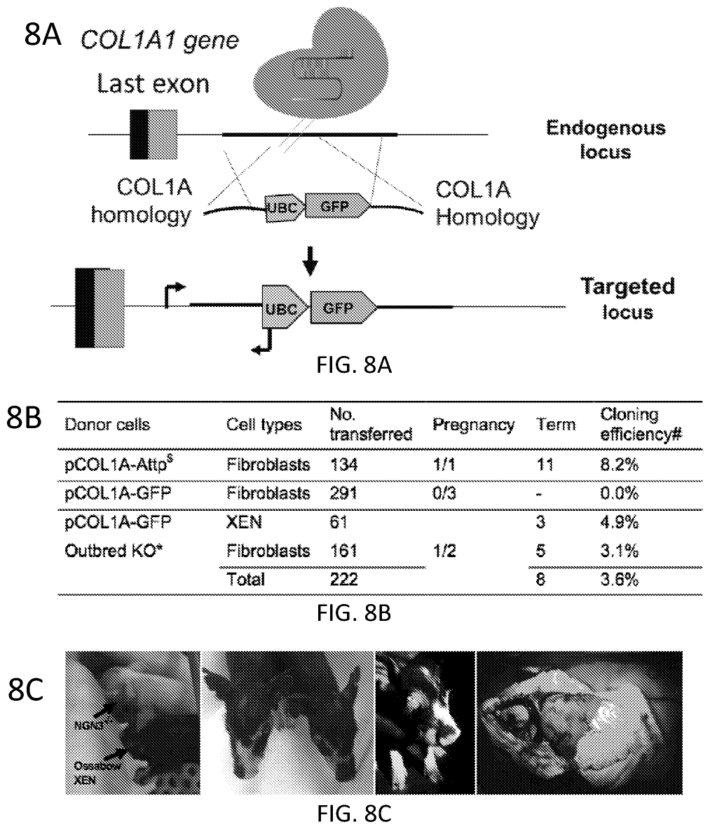

For lineage tracing of injected XEN cells, a total of eight reporter XEN cell lines were established from cloned blastocysts (Day 7 to 8), using GFP KI fetal fibroblasts (pCOL1A-GFP #3 and #6). A candidate female pCOL1a-GFP XEN cell line (Xnt pCOL1A:GFP#3-2) with stable expression of GFP and XEN markers was used for chimera testing. The cells were pre-treated with Rho Kinase (ROCK) inhibitor Y-27632 (10 μM; StemCell Technologies) for 2 hr and dissociated with Accutase at 38.5° C. for 5 min followed by gentle pipetting. About 3-4 small clumps (10-15 cells) were injected per blastocyst (FIG. 3b). After 20-24 hr of culture, injected blastocysts (n=94) were surgically transferred into the upper part of each uterine horn through needle puncture in recipients at days 5-6 of the estrous cycle (D0=onset of estrus: n=3). On day 15 after embryo transfer, the surrogate animals were euthanized to recover XEN-chimeras (XeC; embryonic day 21). A total of 25 fetuses were obtained after transfer and assessed macroscopically for viability and GFP expression. Fetuses that showed strong GFP expression in yolk sac (XeC#3-4) was cut sagittally; one half was used for histological analysis, whereas the second for DNA extraction. For detecting chimera contribution, gDNA were extracted from three parts of embryos: a small pieces of tissue at the posterior region of the fetus, yolk sac, and allantochorionic membrane. Embryos that were malformed or noticeably delayed (i.e. spherical and ovoid) were used only for gDNA isolation. The gDNA samples were subjected to PCR for chimera detection with genotyping primers (Table 4), and qPCR was performed for the detection of knock-in allele and chimerism rate. Prior to use in the qPCR analysis, the dynamic range of qPCR primers were validated (amplification efficiency>90%). The GFP labeled pXEN cell line (Xnt pCOL1A:GFP #3-2) was used as a positive control (GFP+, 100%) and a non-GFP XEN cell from parthenote embryo (Xpg#1) served as a negative (GFP−, 0%) control for investigating % chimerism. Relative expression was calculated using the comparative 2^{-ΔΔ} Ct method. qPCR was performed in triplicate. Cycling conditions for both GFP and reference (ACTB and YWHAZ gene) products were 10 min at 95° C. followed by 40 cycles of 95° C. for 15 sec, and 60° C. for 1 min. The primers used in qPCR are listed in Table 4.

xii. Teratoma Assay

Immunedeficient-nude (BRG, BALB/c-Rag2null IL2rgnull: Taconic) and -scid (NIH-III, Cr:NIH-bg-nu-Xid; National Cancer Institute) male mice were used to perform teratoma formation assay. Before transplanting, the pXEN cells were incubated for 2 hr in DMEM/F12 supplemented with Y27632 (10 M). The cells were dissociated mechanically into small clumps, washed and suspended in 0.2 mL of mixture containing equal volumes of DMEM/F12 and Matrigel (Corning, Mass., USA)14. With six pXEN cell lines, the cell suspensions (1 to 10×106 cells) were subcutaneously injected into 6-8-week-old mice (Table 1). Mice were housed in specific pathogen-free conditions and were monitored for a minimum of 30 weeks.

xiii. Immunofluorescence and Immunohistochemical Analysis

The embryos, explants and derived pXEN cell lines (Xvv#9 and Xnt pCOL1A:GFP#3-2) have been characterized by staining for markers by immunofluorescence (IF) analyses. Samples were fixed with 4% (w/v) paraformaldehyde for 5 min, then washed with DPBS. The sections were permeabilized in DPBS containing 0.01% Triton X-100 (PBT) for 20 min, blocked in blocking solution (10% FBS and 0.05% Triton X-100 in DPBS) for 1 hr, and then incubated with primary antibodies overnight at 4C. The following day, the sections were washed three times in PBT, followed by incubation in the blocking solution with fluorescence labelled secondary antibodies (Alexa Fluor 488 (1:500) and/or Alexa Fluor 568 (1:500) against primary antibody host species) for 1 hr. The cell nuclei were stained with DAPI (Life Technologies) for 5 min in the dark at RT. For Immunohistochemistry, representative samples from the chimeric fetuses including fetal membranes were fixed with 4% formalin overnight at 4° C. Serial paraffin sections were prepared by American Histolabs Inc. (Gaithersburg, Md.) and stained with hemotaxylin and eosin to serve as a reference. Immunostaining was subjected to heat-induced antigen retrieval at 95-98° C. for 20 min in Tris EDTA buffer (pH 9.0, 0.05% Tween20), cooled at RT for 20 min, permeabilized in DPBS containing 0.01% Triton X-100 (PBT) for 20 min, blocked using Super

Block blocking buffer (Thermo Fisher Scientific, Waltham, Mass., USA) for 30 min at RT, and incubated with primary and secondary antibodies and stained using process described above. GFP antibody and IHC protocols were validated with the tissues from a female XEN cloned pig (Xnt clone #1) prior to use in chimera testing. For immunofluorescence and immunohistochemistry, negative control slides, without primary antibody, were included for each experiment to establish background staining. Imaging was performed using an inverted fluorescent microscope (Nikon Eclipse N2000). The source of antibodies used in the experiments were listed in Table 4.

xiv. Statistical Analysis

Statistical analysis was performed with GraphPad Prism 6 (GraphPad Software, Inc., San Diego, Calif., USA) using two-way analysis of variances (ANOVA) and Tukey's multiple comparison test at 5% level of significance. Data were presented as mean SD

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Brandl, U. et al. Transgenic animals in experimental xenotransplantation models: orthotopic heart transplantation in the pig-to-baboon model. Transplant Proc 39, 577-578(2007).
2. Fischer, K. et al. Efficient production of multi-modified pigs for xenotransplantation by ‘combineering’, gene stacking and gene editing. Scientific reports 6, 29081 (2016).
3. Lai, L. & Prather, R. S. Progress in producing knockout models for xenotransplantation by nuclear transfer. Ann Med 34, 501-506 (2002).
4. Niemann, H. & Petersen, B. The production of multi-transgenic pigs: update and perspectives for xenotransplantation. Transgenic Res 25, 361-374 (2016).
5. Piedrahita, J. A. & Mir, B. Cloning and transgenesis in mammals: implications for xenotransplantation. American journal of transplantation: official journal of the American Society of Transplantation and the American Society of Transplant Surgeons 4 Suppl 6, 43-50 (2004).
6. Schuurman, H. J. & Pierson, R. N., 3rd Progress towards clinical xenotransplantation. Front Biosci 13, 204-220 (2008).
7. Shin, J. S. et al. Failure of transplantation tolerance induction by autologous regulatory T cells in the pig-to-non-human primate islet xenotransplantation model. Xenotransplantation 23, 300-309 (2016).
8. Whitelaw, C. B., Sheets, T. P., Lillico, S. G. & Telugu, B. P. Engineering large animal models of human disease. J Pathol 238, 247-256 (2016).
9. Telugu, B. P., Park, K. E. & Park, C. H. Genome editing and genetic engineering in livestock for advancing agricultural and biomedical applications. Mammalian genome: official journal of the International Mammalian Genome Society (2017).
10. Pagliuca, F. W. et al. Generation of functional human pancreatic beta cells in vitro. Cell 159, 428-439 (2014).
11. Yusa, K. et al. Targeted gene correction of alpha-antitrypsin deficiency in induced pluripotent stem cells. Nature 478, 391-394 (2011).
12. Shi, Y., Kirwan, P., Smith, J., Robinson, H. P. & Livesey, F. J. Human cerebral cortex development from pluripotent stem cells to functional excitatory synapses. Nature neuroscience 15, 477-486, S471 (2012).
13. Nakamura, S. et al. Expandable megakaryocyte cell lines enable clinically applicable generation of platelets from human induced pluripotent stem cells. Cell Stem Cell 14, 535-548 (2014).
14. Sun, N. et al. Patient-specific induced pluripotent stem cells as a model for familial dilated cardiomyopathy. Science translational medicine 4, 130ra147 (2012).
15. Volkner, M. et al. Retinal Organoids from Pluripotent Stem Cells Efficiently Recapitulate Retinogenesis. Stem cell reports 6, 525-538 (2016).
16. Lancaster, M. A. et al. Cerebral organoids model human brain development and microcephaly. Nature 501, 373-379 (2013).
17. Spence, J. R. et al. Directed differentiation of human pluripotent stem cells into intestinal tissue in vitro. Nature 470, 105-109 (2011).
18. Takebe, T. et al. Vascularized and functional human liver from an iPSC-derived organ bud transplant. Nature 499, 481-484 (2013).
19. Takasato, M. et al. Directing human embryonic stem cell differentiation towards a renal lineage generates a self-organizing kidney. Nat Cell Biol 16, 118-126 (2014).
20. Rashid, T., Kobayashi, T. & Nakauchi, H. Revisiting the flight of Icarus: making human organs from PSCs with large animal chimeras. Cell Stem Cell 15, 406-409 (2014).
21. Kobayashi, T. et al. Generation of rat pancreas in mouse by interspecific blastocyst injection of pluripotent stem cells. Cell 142, 787-799 (2010).
1. Cockburn, K. & Rossant, J. Making the blastocyst: lessons from the mouse. J Clin Invest 120, 995-1003 (2010).
2. Bauer, M. K. et al. Fetal growth and placental function. Mol Cell Endocrinol 140, 115-120(1998).
3. Carter, A. M. IFPA Senior Award Lecture: Mammalian fetal membranes. Placenta 48 Suppl 1, S21-S30 (2016).
4. Kwon, G. S., Viotti, M. & Hadjantonakis, A. K. The endoderm of the mouse embryo arises by dynamic widespread intercalation of embryonic and extraembryonic lineages. Dev Cell 15, 509-520 (2008).
5. Rossant, J. Stem cells and early lineage development. Cell 132, 527-531 (2008).
6. Fujikura, J. et al. Differentiation of embryonic stem cells is induced by GATA factors. Genes Dev 16, 784-789 (2002).
7. Wamaitha, S. E. et al. Gata6 potently initiates reprograming of pluripotent and differentiated cells to extraembryonic endoderm stem cells. Genes Dev 29, 1239-1255 (2015).
8. McDonald, A. C., Biechele, S., Rossant, J. & Stanford, W. L. Sox17-mediated XEN cell conversion identifies dynamic networks controlling cell-fate decisions in embryo-derived stem cells. Cell reports 9, 780-793 (2014).
9. Cho, L. T. et al. Conversion from mouse embryonic to extra-embryonic endoderm stem cells reveals distinct differentiation capacities of pluripotent stem cell states. Development 139, 2866-2877 (2012).
10. Anderson, K. G. V. et al. Insulin fine-tunes self-renewal pathways governing naive pluripotency and extra-embryonic endoderm. Nat Cell Biol 19, 1164-1177 (2017).
11. Debeb, B. G. et al. Isolation of Oct4-expressing extraembryonic endoderm precursor cell lines. PLoS ONE 4, e7216 (2009).
12. Galat, V. et al. Developmental potential of rat extraembryonic stem cells. Stem Cells Dev 18, 1309-1318 (2009).
13. Lin, J., Khan, M., Zapiec, B. & Mombaerts, P. Efficient derivation of extraembryonic endoderm stem cell lines from mouse postimplantation embryos. Scientific reports 6, 39457 (2016).
14. Seguin, C. A., Draper, J. S., Nagy, A. & Rossant, J. Establishment of endoderm progenitors by SOX transcription factor expression in human embryonic stem cells. Cell Stem Cell 3, 182-195 (2008).
15. Alberio, R., Croxall, N. & Allegrucci, C. Pig epiblast stem cells depend on activin/nodal signaling for pluripotency and self-renewal. Stem Cells Dev 19, 1627-1636 (2010).
16. Telugu, B. P., Ezashi, T. & Roberts, R. M. The promise of stem cell research in pigs and other ungulate species. Stem Cell Rev 6, 31-41 (2010).
17. Keefer, C. L., Pant, D., Blomberg, L. & Talbot, N.C. Challenges and prospects for the establishment of embryonic stem cell lines of domesticated ungulates. Anim Reprod Sci 98, 147-168 (2007).
18. Xue, B. et al. Porcine Pluripotent Stem Cells Derived from IVF Embryos Contribute to Chimeric Development In Vivo. PLoS ONE 11, e0151737 (2016).
19. Hou, D. R. et al. Derivation of Porcine Embryonic Stem-Like Cells from In Vitro-Produced Blastocyst-Stage Embryos. Scientific reports 6, 25838 (2016).
20. Ramos-Ibeas, P., Nichols, J. & Alberio, R. States and Origins of Mammalian Embryonic Pluripotency In Vivo and in a Dish. Current topics in developmental biology 128, 151-179 (2018).
21. Lim, C. Y. et al. Sall4 regulates distinct transcription circuitries in different blastocyst-derived stem cell lineages. Cell Stem Cell 3, 543-554 (2008).
22. Rugg-Gunn, P. J., Cox, B. J., Ralston, A. & Rossant, J. Distinct histone modifications in stem cell lines and tissue lineages from the early mouse embryo. Proc Natl Acad Sci USA 107, 10783-10790(2010).
23. Kunath, T. et al. Imprinted X-inactivation in extra-embryonic endoderm cell lines from mouse blastocysts. Development 132, 1649-1661 (2005).
24. Park, K. E. et al. Targeted Gene Knockin in Porcine Somatic Cells Using CRISPR/Cas Ribonucleoproteins. International journal of molecular sciences 17 (2016).
25. Sheets, T. P. et al. Targeted Mutation of NGN3 Gene Disrupts Pancreatic Endocrine Cell Development in Pigs. Scientific reports 8, 3582 (2018).
26. Chazaud, C., Yamanaka, Y., Pawson, T. & Rossant, J. Early lineage segregation between epiblast and primitive endoderm in mouse blastocysts through the Grb2-MAPK pathway. Dev Cell 10, 615-624 (2006).
27. Plusa, B., Piliszek, A., Frankenberg, S., Artus, J. & Hadjantonakis, A. K. Distinct sequential cell behaviours direct primitive endoderm formation in the mouse blastocyst. Development 135, 3081-3091 (2008).
28. Mitsui, K. et al. The homeoprotein Nanog is required for maintenance of pluripotency in mouse epiblast and ES cells. Cell 113, 631-642 (2003).
29. Gao, Y. et al. Epigenetic regulation of gene expression in porcine epiblast, hypoblast, trophectoderm and epiblast-derived neural progenitor cells. Epigenetics 6, 1149-1161 (2011).
30. Kobayashi, T. et al. Principles of early human development and germ cell program from conserved model systems. Nature 546, 416-420 (2017).
31. Mascetti, V. L. & Pedersen, R. A. Contributions of Mammalian Chimeras to Pluripotent Stem Cell Research. Cell Stem Cell 19, 163-175 (2016).
32. Nagashima, H., Giannakis, C., Ashman, R. J. & Nottle, M. B. Sex differentiation and germ cell production in chimeric pigs produced by inner cell mass injection into blastocysts. Biol Reprod 70, 702-707 (2004).
33. West, F. D. et al. Porcine induced pluripotent stem cells produce chimeric offspring. Stem Cells Dev 19, 1211-1220 (2010).
34. Ezashi, T., Matsuyama, H., Telugu, B. P. & Roberts, R. M. Generation of colonies of induced trophoblast cells during standard reprogramming of porcine fibroblasts to induced pluripotent stem cells. Biol Reprod 85, 779-787 (2011).
35. Fujishiro, S. H. et al. Generation of naive-like porcine-induced pluripotent stem cells capable of contributing to embryonic and fetal development. Stem Cells Dev 22, 473-482 (2013).
36. Zhao, Y. et al. A XEN-like State Bridges Somatic Cells to Pluripotency during Chemical Reprogramming. Cell 163, 1678-1691 (2015).
37. Kobayashi, T. et al. Generation of rat pancreas in mouse by interspecific blastocyst injection of pluripotent stem cells. Cell 142, 787-799 (2010).
38. Matsunari, H. et al. Blastocyst complementation generates exogenic pancreas in vivo in apancreatic cloned pigs. Proc Natl Acad Sci USA 110, 4557-4562 (2013).
39. Wu, J. et al. Interspecies Chimerism with Mammalian Pluripotent Stem Cells. Cell 168, 473-486 e415 (2017).
40. Rashid, T., Kobayashi, T. & Nakauchi, H. Revisiting the flight of Icarus: making human organs from PSCs with large animal chimeras. Cell Stem Cell 15, 406-409 (2014).
41. Masaki, H. et al. Inhibition of Apoptosis Overcomes Stage-Related Compatibility Barriers to Chimera Formation in Mouse Embryos. Cell Stem Cell 19, 587-592 (2016).

1. A method of creating xenotypic organ cells in an animal comprising:
(a) contacting a gene-modifying amino acid sequence and/or gene-modifying nucleic acid sequence with one or a plurality of XEN cells from a first species or one or a plurality of embryos from a second species for a time period sufficient to produce a genetic modification in a genome of the one or a plurality of XEN cells or the one or a plurality of embryos;
(b) injecting the one or a plurality of XEN cells from one species into the one or a plurality of embryos;
(c) implanting the embryo into a female host from the second species to produce a genetically modified fetus.

2. The method of claim 1 further comprising the steps of:
(d) allowing the embryo to develop into a fetus; and
(e) allowing the female host animal to deliver an infant animal comprising the one or a plurality of XEN cells after a period of time sufficient for the fetus to fully develop in the infant animal; or (e) allowing the fetus to develop into an infant animal after a period of time sufficient to remove the fetus surgically from a womb of the female host animal and live ex utero.

3. The method of claim 1 further comprising the step of: screening the one or plurality of XEN cells and/or the one or plurality of embryos for a genetic modification after step (a).

4. The method of claim 2 further comprising the step of:
(f) allowing the infant animal to develop into an adult animal.

5. The method of claim 1, wherein the gene-modifying amino acid sequence comprises one or a combination of functional amino acid sequences selected from: a CRISPR enzyme, TAL nuclease, zinc finger nuclease, and a transposon.

6. The method of claim 1, further comprising the step of:
culturing blastocysts from a zygote of a mammal on feeder cells in culture medium for a time period sufficient to produce the one or a plurality of XEN cells before step (a).

7. A method of producing a germline mutation in the endodermal or mesodermal tissue of a fetus, the method comprising:
(a) injecting one or a plurality of XEN cells and/or XEN-like cells from a first species into an embryo of a second species; and
(b) contacting a gene-modifying amino acid sequence and/or gene-modifying nucleic acid sequence with one or a plurality of XEN cells and/or XEN-like cells; or
contacting a gene-modifying amino acid sequence and/or gene-modifying nucleic acid sequence with one or a plurality of embryos.

8. The method of claim 7 further comprising a step of:
(i) culturing blastocysts from a zygote of a first species on feeder cells in culture medium for a time period sufficient to produce one or a plurality of XEN cells and/or one or a plurality of XEN-like cells before step (a); and/or
(ii) screening the one or plurality of XEN cells, the one or plurality of XEN-like cells, or the embryos for a genetic modification after step (b).

9-11. (canceled)

12. The method of claim 1, wherein the first species is a human and the second species is a horse, cow, goat, sheep, or pig.

13. (canceled)

14. The method of claim 7, wherein the cells or embryo is contacted with a gene-modifying amino acid sequence that comprises a CRISPR enzyme and a gene-modifying nucleic acid sequence that is a guide RNA capable of associating with the CRISPR enzyme.

15. A method of growing a xenotypic organ or organ tissue in an animal comprising:
(a) contacting a gene-modifying amino acid sequence and/or gene-modifying nucleic acid sequence with one or a plurality of mammalian embryos from one species for a time period sufficient to produce a genetic modification in a genome of the one or a plurality of embryos; and
(b) injecting one or a plurality of XEN cells from a second species into an embryo of the first species.

16. The method of claim 15 further comprising:
(c) implanting the embryo into a female host from the first species after performance of step (b).

17. The method of claim 16 further comprising the step of:
(d) allowing a time period to elapse sufficient for an embryo to develop into a fetus within the female host after performance of step (c); and
(e) allowing the female host animal to deliver an infant animal comprising the one or a plurality of XEN cells after a period of time sufficient for the fetus to fully develop as a fetus, or (e) allowing the fetus to develop into an infant animal after a period of time sufficient to remove the fetus surgically from a womb of the female host animal and live ex utero.

18. The method of claim 15 further comprising the step of:
(i) screening the one or plurality of embryos for a genetic modification after step (a); and/or
(ii) culturing blastocysts from a zygote of a mammal on feeder cells in culture medium for a time period sufficient to produce one or a plurality of XEN cells and/or one or a plurality of primary EF cells before step (b).

19. (canceled)

20. The method of claim 15, wherein the gene-modifying amino acid sequence comprises one or a combination of functional amino acid sequences selected from: a CRISPR enzyme, TAL nuclease, zinc finger nuclease, and a transposon.

21. (canceled)

22. The method of claim 15, wherein the xenotypic organ is an organ of endodermal origin, or wherein the xenotypic organ is a human liver or human pancreas.

23. (canceled)

24. The method of claim 15, wherein the female host is a pig and the embryo comprises human XEN cells.

25. A method of microinjecting XEN cells and/or XEN-like cells from a first mammalian species into an embryo of a second mammalian species comprising:
(a) harvesting XEN cells and/or XEN-like cells from a culture;
(b) culturing the embryo; and
(c) injecting the XEN cells and/or XEN-like cells into the embryo.

26. The method of claim 25, wherein the first species is a primate or a human, and wherein the second species is a pig.

27. (canceled)

28. The method of claim 25 further comprising the step of:
(i) culturing the XEN cells and/or XEN-like cells before steps (a) and (c), and wherein, optionally, the XEN cells and/or XEN-like cells are thawed from a frozen state before the step of culturing the XEN cells and/or XEN-like cells; and/or
(ii) modifying the embryo to include at least a first mutation prior to performing step (c), wherein the step of modifying comprises exposing the embryo to one or a combination of functional amino acid sequences selected from: a CRISPR enzyme, TAL nuclease, zinc finger nuclease, and a transposon.

29-31. (canceled)


SEQUENCE LISTING (73 sequences)

SEQ ID NO: 1 - protein, 442 aa, Homo sapiens
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 2 - protein, 457 aa, Homo sapiens
MLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 3 - protein, 595 aa, Homo sapiens
MALTDGGWCLPKRFGAAGADASDSRAFPAREPSTPPSPISSSSSSCSRGGERGPGGASNCGTPQLDTEAAAGPPARSLLLSSYASHPFGAPHGPSAPGVAGPGGNLSSWEDLLLFTDLDQAATASKLLWSSRGAKLSPFAPEQPEEMYQTLAALSSQGPAAYDGAPGGFVHSAAAAAAAAAAASSPVYVPTTRVGSMLPGLPYHLQGSGSGPANHAGGAGAHPGWPQASADSPPYGSGGGAAGGGAAGPGGAGSAAAHVSARFPYSPSPPMANGAAREPGGYAAAGSGGAGGVSGGGSSLAAMGGREPQYSSLSAARPLNGTYHHHHHHHHHHPSPYSPYVGAPLTPAWPAGPFETPVLHSLQSRAGAPLPVPRGPSADLLEDLSESRECVNCGSIQTPLWRRDGTGHYLCNACGLYSKMNGLSRPLIKPQKRVPSSRRLGLSCANCHTTTTTLWRRNAEGEPVCNACGLYMKLHGVPRPLAMKKEGIQTRKRKPKNINKSKTCSGNSNNSIPMTPTSTSSNSDDCSKNTSPTTQPTASGAGAPVMTGAGESTNPENSELKYSGQDGLYIGVSLASPAEVTSSVRPDSWCALALA

SEQ ID NO: 4 - protein, 414 aa, Homo sapiens [sequence omitted]

SEQ ID NO: 5 - protein, 313 aa, Homo sapiens
MYVSYLLDKDVSMYPSSVRHSGGLNLAPQNFVSPPQYPDYGGYHVAAAAAAAANLDSAQSPGPSWPAAYGAPLREDWNGYAPGGAAAAANAVAHGLNGGSPAAAMGYSSPADYHPHHHPHHHPHHPAAAPSCASGLLQTLNPGPPGPAATAAAEQLSPGGQRRNLCEWMRKPAQQSLGSQVKTRTKDKYRVVYTDHQRLELEKEFHYSRYITIRRKAELAATLGLSERQVKIWFQNRRAKERKINKKKLQQQQQQQPPQPPPPPPQPPQPQPGPLRSVPEPLSPVSSLQASVSGSVPGVLGPTGGVLNPTVTQ

SEQ ID NO: 6 - protein, 138 aa, Homo sapiens
KASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 7 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 8 - DNA, 23 bp, Artificial Sequence
  ACTB forward primer
gtggacatcaggaaggacctcta

SEQ ID NO: 9 - DNA, 23 bp, Artificial Sequence
  ACTB reverse primer
gtggacatcaggaaggacctcta

SEQ ID NO: 10 - DNA, 23 bp, Artificial Sequence
  AFP forward primer
gtggacatcaggaaggacctcta

SEQ ID NO: 11 - DNA, 20 bp, Artificial Sequence
  AFP reverse primer
aaggggtgccttcttgctat

SEQ ID NO: 12 - DNA, 20 bp, Artificial Sequence
  CK8 forward primer
tctgggatgcagaacatgag

SEQ ID NO: 13 - DNA, 20 bp, Artificial Sequence
  CK8 reverse primer
ggctgtagttgaagcctgga

SEQ ID NO: 14 - DNA, 20 bp, Artificial Sequence
  CK18 forward primer
gcaagttctgtggacaatgc

SEQ ID NO: 15 - DNA, 20 bp, Artificial Sequence
  CK18 reverse primer
gccagctccgtctcatactt

SEQ ID NO: 16 - DNA, 20 bp, Artificial Sequence
  CK19 forward primer
gccagctccgtctcatactt

SEQ ID NO: 17 - DNA, 20 bp, Artificial Sequence
  CK18 reverse primer
gccagctccgtctcatactt

SEQ ID NO: 18 - DNA, 20 bp, Artificial Sequence
  CK19 forward primer
ctgaaggaagagctggccta

SEQ ID NO: 19 - DNA, 20 bp, Artificial Sequence
  CK19 reverse primer
tcaacctccacactgacctg

SEQ ID NO: 20 - DNA, 20 bp, Artificial Sequence
  CXCR4 5′ primer
cagcaagggtgtgagtttga

SEQ ID NO: 21 - DNA, 20 bp, Artificial Sequence
  CXCR4 3′ primer
tccaaggaaagcgtagagga

SEQ ID NO: 22 - DNA, 20 bp, Artificial Sequence
  cdh1 5′ PRIMER
cacctcacgggaattgtctt

SEQ ID NO: 23 - DNA, 20 bp, Artificial Sequence
  CDH1 3′ primer
ttatcagcacccacgcaata

SEQ ID NO: 24 - DNA, 20 bp, Artificial Sequence
  DKK1 5′ primer
aggctcttggaaccctgact

SEQ ID NO: 25 - DNA, 20 bp, Artificial Sequence
  DKK1 3′ primer
ccaaaggactcaaggcagag

SEQ ID NO: 26 - DNA, 20 bp, Artificial Sequence
  EPCAM 5′ primer
ccaaaaggatggacctgaga

SEQ ID NO: 27 - DNA, 20 bp, Artificial Sequence
  EPCAM 3
agcctgtagaccctgcattg

SEQ ID NO: 28 - DNA, 20 bp, Artificial Sequence
  FOXA2 5′ primer
ataaggagggcaagggaaaa

SEQ ID NO: 29 - DNA, 20 bp, Artificial Sequence
  FOXA2 3′ primer
agtcaaaattcgcaggtgct

SEQ ID NO: 30 - DNA, 20 bp, Artificial Sequence
  GATA4 5′ primer
tctcggaaggcagagagtgt

SEQ ID NO: 31 - DNA, 19 bp, Artificial Sequence
  GATA4 3′ primer
caggcgttgcacaggtagt

SEQ ID NO: 32 - DNA, 20 bp, Artificial Sequence
  GATA6 5′ primer
atcaccatcaccacccaagt

SEQ ID NO: 33 - DNA, 20 bp, Artificial Sequence
  GATA6 3′ primer
cgcgactctgtagactgtgc

SEQ ID NO: 34 - DNA, 19 bp, Artificial Sequence
  GPC1 5′ primer
ccaggatgccagtgatgac

SEQ ID NO: 35 - DNA, 20 bp, Artificial Sequence
  GPC1 3′ primer
tggagcttttcttgctgacc

SEQ ID NO: 36 - DNA, 20 bp, Artificial Sequence
  GSC 5′ primer
gaagccctggagaacctctt

SEQ ID NO: 37 - DNA, 20 bp, Artificial Sequence
  GSC 3′ primer
cggtttttgaaccagacctc

SEQ ID NO: 38 - DNA, 20 bp, Artificial Sequence
  HNF 5′ primer
ctcagcaacggacagatgtg

SEQ ID NO: 39 - DNA, 20 bp, Artificial Sequence
  HNF 3′ primer
caggagcttgtagggctcag

SEQ ID NO: 40 - DNA, 20 bp, Artificial Sequence
  NANOG 5′ primer
cccccttcttcaactcaaca

SEQ ID NO: 41 - DNA, 20 bp, Artificial Sequence
  NANOG 3′ primer
cttcaggcccataaacctca

SEQ ID NO: 42 - DNA, 20 bp, Artificial Sequence
  OCT4 5′ primer
gctggagccgaaccccgagg

SEQ ID NO: 43 - DNA, 25 bp, Artificial Sequence
  OCT4 3′ primer
caccttcccaaagagaacccccaaa

SEQ ID NO: 44 - DNA, 20 bp, Artificial Sequence
  PDGFR 5′ primer
caggttggagggagatggac

SEQ ID NO: 45 - DNA, 18 bp, Artificial Sequence
  PDGF 3′ primer
agttgcggaggttggatt

SEQ ID NO: 46 - DNA, 18 bp, Artificial Sequence
  RSN18 5′ primer
agttgcggaggttggatt

SEQ ID NO: 47 - DNA, 20 bp, Artificial Sequence
  RN18S 3′ primer
cggacacggacaggattgac

SEQ ID NO: 48 - DNA, 20 bp, Artificial Sequence
  SALL4 5′ primer
caggagtaccagagccgaag

SEQ ID NO: 49 - DNA, 20 bp, Artificial Sequence
  SALL4 3′ primer
acctcgggagacttggactt

SEQ ID NO: 50 - DNA, 20 bp, Artificial Sequence
  SNAIL 5′ primer
ttttcagcagccctatgacc

SEQ ID NO: 51 - DNA, 20 bp, Artificial Sequence
  SNAIL 3′ primer
ccaggagagagtcccagatg

SEQ ID NO: 52 - DNA, 20 bp, Artificial Sequence
  SOX2 5′ primer
aacagcccagaccgagttaa

SEQ ID NO: 53 - DNA, 20 bp, Artificial Sequence
  SOX2 3′ primer
gttgtgcatcttggggttct

SEQ ID NO: 54 - DNA, 20 bp, Artificial Sequence
  SOX7 5′ primer
ggctagtgaaagccaactcg

SEQ ID NO: 55 - DNA, 20 bp, Artificial Sequence
  SOX7 3′ primer
tttgcctgccttgagagaat

SEQ ID NO: 56 - DNA, 21 bp, Artificial Sequence
  SOX17 5′ primer
tggttgaatcttgaggtctgc

SEQ ID NO: 57 - DNA, 21 bp, Artificial Sequence
  SOX17 3′ primer
cagggtgtaggtgtgtgatga

SEQ ID NO: 58 - DNA, 20 bp, Artificial Sequence
  SPARC 5′ primer
ggaccatcagtcctctggaa

SEQ ID NO: 59 - DNA, 20 bp, Artificial Sequence
  SPARC 3′ primer
agttctgcgtctcccaaaga

SEQ ID NO: 60 - DNA, 20 bp, Artificial Sequence
  TGF 5′ primer
gtcttcttcggacgttaccg

SEQ ID NO: 61 - DNA, 20 bp, Artificial Sequence
  TGF 3′ primer
gcatgaggaggaggaacaaa

SEQ ID NO: 62 - DNA, 20 bp, Artificial Sequence
  uPA 5′ primer
aagggctctgacattccatg

SEQ ID NO: 63 - DNA, 20 bp, Artificial Sequence
  uPA 3′ primer
ccggctcttacactgacaca

SEQ ID NO: 64 - DNA, 20 bp, Artificial Sequence
  vimentin 5′ primer
gtaccggagacaggtgcagt

SEQ ID NO: 65 - DNA, 20 bp, Artificial Sequence
  vimentin 3′ primer
ttccacggcaaagttctctt

SEQ ID NO: 66 - DNA, 19 bp, Artificial Sequence
  GFP 5′ primer
aagttcatctgcaccaccg

SEQ ID NO: 67 - DNA, 20 bp, Artificial Sequence
  GFP 3′ primer
tccttgaagaagatggtgcg

SEQ ID NO: 68 - DNA, 22 bp, Artificial Sequence
  YWHAZ 5′ primer
agtaggttgggctccttgacac

SEQ ID NO: 69 - DNA, 22 bp, Artificial Sequence
  YWHAZ 3′ primer
gccgactgtgactttaaggtgc

SEQ ID NO: 70 - DNA, 20 bp, Artificial Sequence
  pCOL 5′ primer
gcatggagagaaggcatgat

SEQ ID NO: 71 - DNA, 20 bp, Artificial Sequence
  UBC 3′ primer
tcacagcgatccagaaagaa

SEQ ID NO: 72 - DNA, 20 bp, Artificial Sequence
  NGN3 5 ′ primer
caccagaccgagcagtcttt

SEQ ID NO: 73 - DNA, 19 bp, Artificial Sequence
  NGN3 3′ primer
ttggtgagtttcgcatcgt